(19) United States
(12) Patent Application Publication
(10) Pub. No.: US 2021/0054049 A1
(43) Pub. Date: Feb. 25, 2021

(54) VARIANT DOMAINS FOR MULTIMERIZING PROTEINS AND SEPARATION THEREOF

(71) Applicant: Merus N.V., Utrecht (NL)

(72) Inventors: Cornelis Adriaan DE KRUIF, Utrecht (NL); Peter Brian SILVERMAN, Utrecht (NL); Richard BONNEAU, Utrecht (NL)

(21) Appl. No.: 16/870,715

(22) Filed: May 8, 2020

(30) Foreign Application Priority Data
May 9, 2019 (EP) 19173633.9

(51) Int. Cl. C07K 16/00; C07K 1/18

(52) U.S. Cl. CPC C07K 16/00; C07K 1/18; C07K 2317/526; C07K 2317/522; C07K 2317/524; C07K 2317/31

(57) ABSTRACT

The current invention relates to means and methods for producing and isolating immunoglobulin proteins comprising a first and a second immunoglobulin polypeptide, in particular to means and methods for producing, and separating proteins comprising said first and second immunoglobulin polypeptide. By including variations to amino acids, and variant separation domains from a cell producing the desired immunoglobulin protein, a desired immunoglobulin protein as produced can be separated from mixtures of immunoglobulin proteins.

### CROSS-REFERENCE TO RELATED APPLICATIONS

This application claims priority to EP Application No. 19173633.9, filed May 9, 2019. The entire contents of EP Application No. 19173633.9 are hereby incorporated herein by reference.

### REFERENCE TO A SEQUENCE LISTING SUBMITTED ELECTRONICALLY VIA EFS-WEB

This application includes a Sequence Listing submitted electronically via EFS-Web (name: “4096_0320001_Seqlisting_ST25”; size: 56,192 bytes; and created on: May 8, 2020), which is hereby incorporated by reference in its entirety.

### INTRODUCTION

An important class of therapeutic molecules of the last decades has been the class of monoclonal antibodies. Monoclonal antibodies have been successful for treatment of a variety of diseases, including cancer. Over the last decade it has been found that targeting more than one epitope, for instance more than one epitope on a tumor cell can also be efficacious. Patients can be given combinations of monoclonal antibodies that were separately developed, but also combinations of monoclonal antibodies from one cell. Such cells can produce antibodies with two or more different specificities which form part of a mixture of antibodies developed for the purpose of targeting more than one target on one or more cell types. When two antibodies are expressed in one cell various combinations of antibodies can be produced, including comprising bispecific and monospecific antibodies.

Techniques are available to tailor the association of various immunoglobulin chains. Various dimerization domains have been developed to favor certain associations of heavy chains in such producing cells. A common light chain can be used to avoid mispairing of cognate heavy and light chains. Bispecific antibodies can in certain applications replace the use of combinations of two antibodies. Bispecifics can also be used to bring together two cells in a subject, e.g. a tumor cell and an immune cell, such as a T-cell. An example thereof is the combined targeting of CD3 and epitopes present on cancer cells. Similarly, multivalent multimers or multispecific antibodies, capable of binding three or more of the same or different antigens or epitopes have emerged. Whereas a combination of two antibodies represents a mixture of two different immunoglobulins that bind to different epitopes on the same or different targets, in a bispecific antibody this is achieved through a single immunoglobulin. In a multispecific multimer or multispecific antibody, three or more different epitopes on the same or different antigens may be targeted.

By binding to two epitopes on the same or different targets, bispecific antibodies can have similar or superior effects as compared to a combination of two antibodies binding to the same epitopes. This also applies to multispecific multimers, capable of binding three or more targets. Bispecific or multispecific immunoglobulin proteins may cluster two or more surface proteins on a cell or may bring an immune effector cell in proximity to an aberrant cell, in either case causing apoptosis of the cell. Furthermore, isolated bispecific antibodies combining two different binding regions in a single molecule have also shown advantageous effects over mixtures of two antibodies targeting the same two different targets. From a technological and regulatory perspective, development of a single bispecific antibody or multispecific multimer or antibody can be less complex because manufacturing, preclinical and clinical testing involves a single molecule. Thus, therapies based on a bispecific antibody or multispecific multimer/antibody can be facilitated by a less complicated and cost-effective drug development process while having concomitantly the potential to provide for more efficacious therapies.

Bispecific antibodies such as those based on the IgG formats have been produced by a variety of methods. For instance, bispecific antibodies may be produced by expressing the components of two antibodies in a single cell using recombinant DNA technology. As stated herein before, these approaches can, in some embodiments, yield multiple antibody species, for instance, where two different heavy chains and two different light chains are produced by the cell. Unless specifically tailored, a heavy chain can typically pair with any light chain that is produced by the cell, typically leading to non-functional binding sites if they are not the right cognate pairs. In the above example one such heavy chain might pair with either light chain.

Unless specifically tailored, a heavy chain can typically pair with any other heavy chain that is produced by the cell. In an untailored setting, up to ten different immunoglobulin molecules can be produced by the cell. The complexity of the antibody mixture and the presence of non-functional heavy and light chain combination can be addressed by selecting heavy-light chain combinations that share a common light chain. This applies also to the production of multispecific multimers or antibodies and situations where three or more variable domains are incorporated into a single antibody using recombinant DNA technology.

When a common light chain is used, combined with expression of two or more heavy chains that contain modifications that drive specific heterodimerization of the different heavy chains by a single producer cell, some homodimers of paired heavy chains having the same binding domain may nonetheless be produced resulting in a mixture of monospecific and bispecific antibodies. This also applies when using a common light chain, combined with the expression of two or more heavy chains and one or more of such heavy chains contain two or more heavy chain variable regions, such that a single producer cell may produce a multispecific multimer or antibody, and additional homodimers. In cases where specific homodimers are desired, some heterodimers may be produced. Accordingly, in each circumstance where a mixture of protein is produced, a desired dimer(s) may need to be isolated from the resulting mixture. Hence, there is a need in the art for improved and/or alternative technologies for producing and separating monospecific or bispecific antibodies, or multivalent antibodies or multimers.

Various separation methods are available that utilize the charge and/or the isoelectric point (pI) of antibodies or fragments thereof or that make use of isoelectric focusing or resulting unique peaks of desired protein species that occurs through charge chromatography. Herein, new products are disclosed that facilitate separation from mixtures as well as new methods to separate such products.

### EMBODIMENTS

Where charged amino acids are referred to herein, they refer to charges at physiological relevant pH, including for example under in vivo conditions.

In one embodiment the invention provides an immunoglobulin region, preferably a CH1 region comprising a variation from an amino acid as compared to an original immunoglobulin region, preferably an original CH1 region, and more preferably a human wild-type CH1 region, wherein the original amino acid is not surface exposed in the original immunoglobulin region, wherein the variation is selected from
a neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid.

In one embodiment the invention provides an immunoglobulin region, preferably an immunoglobulin CH1, CH2, CH3 region comprising a variation of an amino acid as compared to an original of said immunoglobulin region, preferably an original CH1, CH2 or CH3 region, and more preferably a human wild-type CH1, CH2 or CH3 region that is non-surface exposed in an immunoglobulin or a combination of said regions, wherein the variation is selected from
a neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid.

The immunoglobulin CH1 region in accordance with the invention preferably comprises one or more variations from one or more non-surface exposed or preferably buried amino acids as compared to a human wild-type CH1 region, selected from the group consisting of:
a variation of a neutral amino acid to a negatively charged amino acid;
a variation of a positively charged amino acid to a neutral amino acid;
a variation of a neutral amino acid to a positively charged amino acid; and
a variation of a negatively charged amino acid to a neutral amino acid.

The invention also provides an immunoglobulin CH1 region comprising a variation of an amino acid as compared to a human wild-type CH1 region, which is at a position (EU numbering) selected from N159, N201, T120, K147, D148, Y149, V154, A172, Q175, S190, and K213. The variation of an amino acid is preferably at positions from D148, Y149, V154, N159, A172, S190, and N201. In a preferred embodiment the variation is at a position of an amino acid selected from N159 and/or N201. The CH1 region may comprise two or more variations of said amino acids. Said two or more variations preferably comprise two or more of a
a neutral amino acid to a negatively charged amino acid;
a positively charged amino acid to a neutral amino acid;
a positively charged amino acid to a negatively charged amino acid;

or two or more of a
a neutral amino acid to a positively charged amino acid;
a negatively charged amino acid to a neutral amino acid; and
a negatively charged amino acid to a positively charged amino acid.
   Suitable combinations of two or more variations in a CH1 region comprise variations of amino acids selected from the group A172/S190/N201, T197/K213, D148/Q175, N159/Q213, K147/Q175, Y149/V154/A172/S190, N201/K213, T120/N201, N201/N159, T120/N159, T120/N201/N159 and N201/K213/N159.

The invention also provides an immunoglobulin CH2 region comprising a variation of an amino acid as compared to a human wild-type CH2 region, which is at a position (EU numbering) V303. The immunoglobulin CH2 region is in one embodiment an Fc-silent CH2 region, preferably comprising an L235G and an G236R amino acid variation.

The invention also provides an immunoglobulin CH3 region comprising a variation of one or more amino acids as compared to a human wild-type CH3 region, which is/are at position (EU numbering) K370, E382 and/or E388. The immunoglobulin CH3 region in one embodiment comprises residue variations for the promotion of heterodimerization at the CH3/CH3 interface, preferably comprising a L351D and L368E variation or alternatively comprising a T366K and L351K variation.

In one embodiment the immunoglobulin CH1, CH2, CH3 region or combination thereof comprises two or more variations of amino acids of which at least one is a variation of an amino acid that is not surface exposed in an immunoglobulin. In one embodiment the immunoglobulin CH1, CH2, CH3 region or combination thereof comprises two or more variations of amino acids that are not surface exposed in an immunoglobulin. A variation is preferably selected from
a neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid. The one or more variations preferably comprises one or more variations of one or more non-surface exposed or preferably buried amino acids selected from the group consisting of:
a variation of a neutral amino acid to a negatively charged amino acid;
a variation of a positively charged amino acid to a neutral amino acid;
a variation of a neutral amino acid to a positively charged amino acid; and
a variation of a negatively charged amino acid to a neutral amino acid. The at least one is a variation of an amino acid is preferably a buried amino acid.

A CH region comprising a variation of a neutral amino acid to a negatively charged amino acid; a positively charged amino acid to a neutral amino acid; and/or a positively charged amino acid to a negatively charged amino acid is said to be a CH region with a negative charge difference with respect to the original CH region, preferably as compared to a human wild-type CH region. The variation itself is said to provide the negative charge difference to the CH region. A CH region with a variation of a neutral amino acid to a positively charged amino acid; a negatively charged amino acid to a neutral amino acid; and/or a negatively charged amino acid to a positively charged amino acid is said to be a CH region with a positive charge difference with respect to the original CH region, preferably as compared to a human wild-type CH region. If a CH region has two variations of an amino acid residue as described herein it is preferred that both variations provide the same charge difference in kind to the CH region. If a CH region has three or more variations of an amino acid residue as described herein it is preferred that net result of the variations provide a charge difference to the CH region.

The immunoglobulin region is preferably a human immunoglobulin region. In some embodiments the immunoglobulin region is an IgG region, preferably an Ig1 region. The immunoglobulin regions disclosed above can for instance be used advantageously as a part of an antibody that needs to be separated from a mixture of antibodies.

The invention further provides an antibody comprising a heavy chain and a light chain comprising an immunoglobulin CH region as described herein. For instance when such an antibody is produced as part of a mixture, the variation in charge provided to a CH region may facilitate separation of said antibody from said mixture. In a preferred embodiment the antibody comprises different heavy chains. In a preferred embodiment the antibody is a multispecific antibody such as a bispecific or trispecific antibody. In this case the variation in charge provided to a CH region may facilitate separation of said bispecific or trispecific antibody from said mixture. The different heavy chains preferably comprise compatible heterodimerization regions, preferably compatible heterodimerization CH3 regions. In one embodiment one of heavy chains comprises the CH3 variations L351D and L368E, and the other of said heavy chains comprises the CH3 variations T366K and L351K. The antibody is preferably an IgG antibody, preferably an IgG1 antibody. In some embodiments the antibody comprises a first and a second heavy chain that each comprises one or more of the immunoglobulin CH regions as described herein. It is preferred that the heavy chain that comprises the CH3 variations L351D and L368E comprises one CH region as described herein and that the heavy chain that comprises the CH3 variations T366K and L351K comprises another CH region as described herein. In such cases it is preferred that the one and the other CH regions comprise CH regions with different charges. In such cases the difference in iso-electric points of the resulting antibodies in the mixture will be further apart thereby facilitating separation of said antibody from other immunoglobulin molecules or parts thereof in said mixture. In other words if one CH region is a CH region with a negative charge difference with respect to the original CH region the other is preferably a CH region with a positive charge difference with respect to the original CH region. Similarly if one CH region is a CH region with a positive charge difference with respect to the original CH region the other is preferably a CH region with a negative charge difference with respect to the original CH region.

Antibodies with compatible heterodimerization regions such as compatible CH3 heterodimerization regions as described herein that have a CH region as described herein typically separate better from the respective antibodies having the same heavy chains, and/or half antibodies, if present, in a separation step that utilize charge and/or the isoelectric point (pI) of antibodies or fragments thereof. The antibody preferably comprises one or more light chains. It preferably comprises the same light chain. The light chain is preferably a common antibody light chain as described herein. The common light chain preferably comprises a light chain variable region of FIG. 13A-13E, for example of FIG. 13B or FIG. 13D. In one embodiment the light chain has a light chain constant region as depicted in FIG. 13C. In a preferred embodiment the light chain has an amino acid sequence of a light chain depicted in FIG. 13A or FIG. 13E. In a preferred embodiment the light chain has an amino acid sequence of the light chain depicted in FIG. 13A. A common light is preferably a light chain having the CDRs as depicted in FIG. 13F.

An antibody, CH region or CH domain as described herein is preferably a human antibody or human immunoglobulin CH region or domain. It is preferably a human antibody. CH domain or CH region which comprising a CH region with a variation at an amino acid position that is non-surface exposed, and preferably buried within a wild-type human CH region.

The immunoglobulin region, preferably a CH region or antibody comprising a variation of an amino acid that is not surface exposed as described herein, preferably has a variation that is selected from amino acids that are not present at the CH1/CL interface, not present at the CH2/CH2 interface and/or not present at the CH3/CH3 interface, CH3/CH3 interface amino acids are listed in FIG. 22 according to Traxlmayer et al (2012; J Mol Biol. October 26; 423(3): 397-412. discussion and FIG. 3).

The immunoglobulin region, preferably a CH1, CH2 or CH3 region or antibody comprising a variation of an amino acid that is not surface exposed as described herein, which does not substantially, adversely affect the stability of the resulting CH1/CL domain, CH2 domain or CH3 domain or antibody, including any heavy and light chain interface. The immunoglobulin region, preferably a CH1, CH2 or CH3 region or antibody comprising a variation of an amino acid that is not surface exposed as described herein, may include additional variation(s) that bolster stability of the variation(s) that produce a charge difference. The immunoglobulin region, preferably a CH1, CH2 or CH3 region or antibody comprising a variation of an amino acid that is not surface exposed as described herein, may include additional variation(s) that produce a charge difference. The invention also provides an immunoglobulin CH1/CL domain, a CH2 domain or CH3 domain comprising an immunoglobulin region as described herein. A CH2 domain may further comprise an Fc-silent mutation, preferably comprising the CH3 variations at 235 and/or 236. A CH3 domain may further comprise a CH3 heterodimerization domain, preferably comprising the CH3 variations L351D and L368E in one CH3 region, and the CH3 variations T366K and L351K on the other.

The invention further provides a protein comprising one or more CH1, CH2, CH3 regions or combinations thereof as described herein. Also provided is a protein comprising one or more CH1/CL, CH2, CH3 domains or combinations thereof as described herein.

The invention further provides an antibody, preferably a multispecific antibody such as bispecific antibody comprising one or more CH1/CL, CH2, CH3 domains or combinations thereof as described herein.

Two or more variations in a CH1, CH2, CH3 region or combinations thereof in one chain of an immunoglobulin, polypeptide or protein preferably all comprise variations that direct the charge in the same direction. i.e. all towards a more positive charge of the CH region(s) or combination thereof, or all towards a more negative charge of the CH region(s) or combination thereof.

The invention further provides a composition comprising the immunoglobulin region or antibody as described herein and a pharmaceutical carrier or pharmaceutical excipient. Further provided is a pharmaceutical composition comprising the immunoglobulin region or antibody as described herein. The pharmaceutical composition preferably comprises a pharmaceutical carrier or pharmaceutical excipient.

Further provided is a nucleic acid that encodes the immunoglobulin region or antibody as described herein. Further provided is a combination of nucleic acids that together encode the antibody or multimeric protein incorporating the immunoglobulin region as described herein. The nucleic acids may be physically linked or not.

Also provided is a recombinant host cell comprising the nucleic acid or combination of nucleic acids.

The invention further provides a method of producing an antibody of the claims, wherein the method comprises the steps of

providing a nucleic acid encoding a first heavy chain with a CH1, CH2, CH3 region or combination thereof as described herein;
providing a nucleic acid encoding a second heavy chain, wherein said first and second heavy chain may be the same or different;
providing a nucleic acid encoding a light chain;
introducing said nucleic acid into host cells and culturing said host cells to express the nucleic acid(s); and
collecting the antibody from the host cell culture, the method further comprising separation of the antibody from other antibodies or antibody fragments in a separation step based on the electrical charge of the antibodies and/or antibody fragments. In one embodiment said first and second heavy chains comprise compatible heterodimerization regions, preferably compatible CH3 heterodimerization regions.

The invention further provides a method of producing an antibody of the claims, wherein the method comprises the steps of

providing a nucleic acid encoding a first heavy chain with a CH1, CH2, CH3 region or combination thereof as described herein:
providing a nucleic acid encoding a second heavy chain, wherein said first and second heavy chain may be the same or different;
providing a nucleic acid encoding a light chain;
introducing said nucleic acid into host cells and culturing said host cells to express the nucleic acid(s); and
collecting the antibody from the host cell culture, the method further comprising performing a harvest clarification,
performing protein capture,
performing anion exchange chromatography, and
performing cation exchange chromatography to separate the antibody from other antibodies or antibody fragments. In one embodiment said first and second heavy chains comprise compatible heterodimerization regions, preferably compatible CH3 heterodimerization regions.

The invention further provides a method of producing an antibody of the claims, wherein the method comprises the steps of

providing a nucleic acid encoding a first heavy chain with a CH1, CH2, CH3 region or combination thereof as described herein;
providing a nucleic acid encoding a second heavy chain, wherein said first and second heavy chain may be the same or different;
providing a nucleic acid encoding a light chain;
introducing said nucleic acid into host cells and culturing said host cells to express the nucleic acid(s); and
collecting the antibody from the host cell culture, the method further comprising separating the antibody from other antibodies or antibody fragments in a separation step comprising isoelectric focusing on a gel.

Further provided is a method for producing a multispecific antibody comprising a first heavy chain and a second heavy chain whose isoelectric points are different, wherein the method comprises the steps of:

(a) expressing a nucleic acid encoding a first heavy chain and a nucleic acid encoding a second heavy chain, such that isoelectric points of the encoded first heavy chain and that of the encoded second heavy chain differ, wherein said nucleic acid encodes one or more variations at amino acid position(s) selected from non-surface exposed positions of an encoded immunoglobulin region comprising a first and/or second heavy chain, preferably a CH1 region, more preferably. T120, K147, D148, Y149, V154, N159, A172, Q175, S190, N201, and K213, and/or preferably a CH2 region, preferably a V303, and/or preferably a CH3 region, preferably a K370, an E382, an E388 (EU-numbering) and
(b) culturing host cells to express the nucleic acid; and
(c) collecting the multispecific antibody from the host cell culture, using the difference in isoelectric point.

Also provided is a method for separating a multispecific antibody comprising a first heavy chain and a second heavy chain whose isoelectric points are different, wherein the method comprises the steps of:

(a) expressing both or either one of a nucleic acid encoding the amino acid residues of the first heavy chain and a nucleic acid encoding the amino acid residues of the second heavy chain, such that the isoelectric point of the encoded first heavy chain and that of the encoded second heavy chain differ, wherein the position(s) of said nucleic acid is/are position(s) that differ from an encoded CH1, CH2, CH3 region or combination thereof at a non-surface exposed residue(s), preferably one or more amino acid variations selected from T120, K147, D148, Y149, V154, N159, A172, Q175, S190, N201, and K213, and/or preferably a (12 region, preferably a V303, and/or preferably a CH3 region, preferably a K370, an E382, an E388 (EU-numbering) and
(b) culturing host cells to express the nucleic acid; and
(c) separating the multispecific antibody from the host cell culture by chromatography.

In a preferred embodiment the nucleic acid encodes a first heavy chain and second heavy chain, such that a retention time of the first heavy chain, a homomultimer of the first heavy chain, the second heavy chain, a homomultimer of the second heavy chain, and a heteromultimer of the first and second heavy chain differ when expressed and are separated in an ion exchange chromatography step.

The variant amino acid(s) at the position(s) encoded by said nucleic acid is/are preferably selected from amino acids that are non-surface exposed in a human wild-type CH1, CH2, CH3 region or combination thereof and selected from
a neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid.

Also provided is a method for producing a multispecific antibody comprising a first heavy chain and a second heavy chain whose isoelectric points are different, wherein the method comprises the steps of:
providing a nucleic acid encoding a CH1, CH2, CH3 region or combination thereof of the first heavy chain and a nucleic acid encoding a CH1, CH2, CH3 region or combination thereof of the second heavy chain, such that the isoelectric point of the first encoded heavy chain and that of the second encoded heavy chain differ, wherein at least one of said CH regions comprises an amino acid variation at a position selected from T120, K147, D148, Y149, V154, N159, A172, Q175, S190, N201, K213, V303, K370, E382 and E388 (EU-numbering) and
culturing host cells to express the nucleic acid; and
collecting the multispecific antibody from the host cell culture, using the difference in isoelectric point further comprising the steps of
collecting the antibody from the host cell culture,
performing harvest clarification.
performing protein capture,
performing anion exchange chromatography, and
performing cation exchange chromatography to separate the antibody from another antibody or an antibody fragment.

Further provided is a method for purifying a multispecific antibody comprising a first heavy chain and a second heavy chain whose isoelectric points are different, wherein the method comprises the steps of:
providing both or either one of a nucleic acid encoding a CH1, CH2, CH3 region or combination thereof of the first heavy chain and a nucleic acid encoding a CH1, CH2, CH3 region or combination thereof of the second heavy chain, such that the first encoded heavy chain and the second encoded heavy chain differ in isoelectric point, wherein at least one of said CH1 regions comprises an amino acid variation at a position selected from T120, K147, D148, Y149, V154, N159, A172, Q175, S190, N201, K213, V303, K370, E382 and E388 (EU-numbering) and
culturing host cells to express the nucleic acid; and
purifying the multispecific antibody from the host cell culture by isoelectric focusing and separating the multispecific antibody from another antibodies or an antibody fragment.

The one or more nucleic acid encoding a homomultimer of the first heavy chain, a homomultimer of the second heavy chain, and a heteromultimer of the first and second heavy chain are expressed as proteins having different isoelectric points and produce different retention times in ion exchange chromatography.

The position(s) of said one or more amino acid variations of a CH region are preferably non-surface exposed in the multispecific antibody and are preferably selected from
a neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid.

The amino acids at the variant positions preferably comprises one or more variations of one or more non-surface exposed or preferably buried amino acids selected from the group consisting of:
a neutral amino acid to a negatively charged amino acid;
a positively charged amino acid to a neutral amino acid;
a neutral amino acid to a positively charged amino acid; and
a negatively charged amino acid to a neutral amino acid. The first heavy chain and the second heavy chain preferably comprise CH3 regions and said CH3 regions preferably comprise compatible CH3 heterodimerization regions. One of said compatible CH3 heterodimerization regions preferably comprises an L351D and L368E and the other preferably comprises a T366K and L351K.
   The variant amino acid(s) at the position(s) encoded by said nucleic acid is/are preferably selected from T120, K147, D148, Y149, V154, N159, A172, Q175, S190, N201, K213, V303, K370, E382 and E388.

Further provided is a CH1 region or CH1-containing immunoglobulin polypeptide comprising a first charged amino acid residue at non-surface exposed positions in a human, wild-type CH1, preferably at position 120, position 147, position 148, position 149, position 154, position 159, position 172, position 175, position 190, position 201, or position 213. The CH region or CH1-containing immunoglobulin polypeptide preferably comprises in addition to the charged residue a second charged amino acid residue at a different position selected from a non-surface exposed position in a human, wild-type CH1, preferably at position 120, position 147, position 148, position 149, position 154, position 159, position 172, position 175, position 190, position 201, or position 213, said second charged amino acid having the same charge as the first charged amino acid. The CH1 region or CH1-containing immunoglobulin polypeptide preferably comprises a neutral or a negatively charged amino acid residue at position 147 and/or position 213. The CH1 region or CH1-containing immunoglobulin polypeptide preferably comprises a neutral or a positively charged amino acid residue at position 148 and/or at the hinge at position 216. Further provided is a CH2 region or CH2-containing immunoglobulin polypeptide comprising a charged amino acid residue at a non-surface exposed position in a human, wild-type CH2, preferably at position 303. Further provided is a CH3 region or CH3-containing immunoglobulin polypeptide comprising a first neutral amino acid residue at non-surface exposed positions in a human, wild-type CH3, preferably at position 370, position 382 or position 388. The CH3 region or CH3-containing immunoglobulin polypeptide preferably comprises in addition to the neutral residue a second neutral amino acid residue at a different position selected from a non-surface exposed position in a human, wild-type CH3 preferably at position 370, position 382 or position 388, different from the position of the first neutral amino acid. Alternatively, provided is a CH3 region or CH3-containing immunoglobulin polypeptide comprising a first negative amino acid residue at non-surface exposed positions in a human, wild-type CH3, preferably at position 370, and a positive amino acid at position 382 or position 388.

A variation at position T120 of a CH1 region is preferably a variation of a neutral amino acid to a charged amino acid. Examples are T120R, T120K, T120D and T120E variations. The variation preferably comprises a T120D or a T120K variation.

A variation at position K147 of a CH1 region is preferably a variation of a positive charged amino acid to a neutral or negative amino acid. Examples are K147Q, K147T, K147S, K147D and K147E variations. The variation is preferably a K147E variation.

A variation at position D148 of a CH1 region is preferably a variation of a neutral amino acid to a charged amino acid. Examples are D148R, D148K, D148D and D148E variations. The variation preferably comprises a D148K variation.

A variation at position N159 of a CH1 region is preferably a variation of a neutral amino acid to a charged amino acid. Examples are N159R, N159K, N159D and N159E variations. The variation preferably comprises a N159K or a N159D variation.

A variation at position Q175 of a CH1 region is preferably a variation of a neutral amino acid to a charged amino acid. Examples are Q175R, Q17K, Q175D and Q175E variations. The variation preferably comprises a Q175K or a Q175E variation.

A variation at position N201 of a CH1 region is preferably a variation of a neutral amino acid to a charged amino acid. Examples are N201R, N201K, N201D and N201E variations. The variation preferably comprises a N201K or a N201D variation.

A variation at position K213 of a CH1 region is preferably a variation of a positive charged amino acid to a neutral or negative amino acid. Examples are K213Q, K213T, K213S, K213D and K213E variations. The variation preferably comprises a K213Q variation.

A variation at position V303 of a CH2 region is preferably a variation of a neutral to a charged amino acid. Examples are V303K V303R, V303D, and V303E variations. The variation preferably comprises a V303D or a V303E variation.

Further provided is a CH2-containing immunoglobulin polypeptide comprising a charged amino acid residue at position 303.

Also provided is a CH3-containing immunoglobulin polypeptide comprising a non-charged amino acid residue at a position selected from position 370, position 382, or position 388.

The CH2- and/or CH3-containing immunoglobulin polypeptide as described herein, may comprises two or more of the amino acid variations selected from a charged amino acid residue at position 303, or a non-charged amino acid residue at position 370, position 382, or position 388.

A CH2 region variation as indicated herein is preferably a CH2 variation at position V303. The variation is preferably a variation of a neutral amino acid to a charged amino acid. Examples are a V303R, V303K, V303D or V303E variation. A preferred variation is a V303K variation or a V303E variation as described in the examples.

A CH3 region variation as indicated herein is preferably a CH3 variation at position K370, E382, E388 or a combination thereof. The variation at position K370 is preferably a variation of a charged amino acid to a neutral amino acid. Examples are a K370Q, a K370N, a K370H, a K370S, a K370T, or K370Y variation. A preferred variation is a K370S or a K370T variation as described in the examples. The variation at position E382 is preferably a variation of a charged amino acid to a neutral amino acid. Examples are an E382Q, an E382N, an E382H, an E382S, an E382T, or an E382Y variation. A preferred variation is an E382Q or an E382T variation as described in the examples. The variation at position E388 is preferably a variation of a charged amino acid to a neutral amino acid. Examples are an E388Q, an E388N, an E388L, an E388S, an E388T, or an E388M variation. A preferred variation is an E388L, an E388M or an E388T variation as described in the examples.

The immunoglobulin polypeptide as described herein is preferably an antibody, preferably a multispecific antibody.

The antibody may further comprise a positively charged amino acid residue at a hinge position 216.

The antibody may further comprise a variation at an amino acid selected from T197 and at a hinge position E216.

Also provided is a composition comprising the immunoglobulin domain, immunoglobulin region polypeptide, protein or antibody as described herein which further comprises one or more of the following variations G122P, I199V, N203I, S207T, and V211I in the CH1 domain.

The invention may be used to provide separation between antibodies or immunoglobulin proteins as described herein, between a bispecific antibody and monospecific antibodies as described herein, between a multispecific antibody and other multispecific and monospecific antibodies and half antibodies as described herein.

The invention may also be used to optimize co-purification of two or more desired antibodies produced by a cell. For instance, by providing three or more heavy chains that can pair with a common light chain, and wherein one of said heavy chains has a member of a compatible heterodimerization domain, and the other heavy chains have the other member of the compatible heterodimerization domain, for instance a CH3 DE region in one and a CH3 KK region in the others, two or more bispecific antibodies may be produced. Tailoring the charge of one or more of the heavy chains according to the invention can provide heterodimeric heavy chain containing antibodies that co-migrate in a separation method that utilize charge and/or the pI. The charge can be tailored such that the co-migrating heterodimeric heavy chain containing antibodies migrate at a different position than the respective monomeric heavy chain containing antibodies and/or half-antibodies.

Further provided is an immunoglobulin protein comprising a first CH1 region or CH1-containing immunoglobulin polypeptide and a second CH1 region or CH1-containing immunoglobulin polypeptide, wherein the first and/or second CH1 region or CH1-containing immunoglobulin polypeptides comprise one or more variations of one or more amino acids selected from amino acids within the CH1 region that are non-surface exposed, such that the isoelectric point of the immunoglobulin protein comprising the first CH1 region or CH1-containing immunoglobulin polypeptide and the second CH1 region or CH1-containing immunoglobulin polypeptide is different from the isoelectric points of immunoglobulin proteins containing only the first CH1 region or CH1-immunoglobulin polypeptide or immunoglobulin proteins containing only the second CH1 region or CH1-immunoglobulin polypeptide.

In one embodiment the invention relates to proteins comprising at least two different polypeptides comprising a heavy chain domain, such as e.g. bispecific antibodies or multivalent multimers comprising e.g. at least two different heavy chain variable regions and a common light chain. The invention further relates to the means and methods of producing and separating such proteins. Proteins comprising two different immunoglobulin variable region polypeptides are generally referred to herein as bispecific proteins, bispecific immunoglobulins or bispecific antibodies. Based on a format of proteins comprising two different immunoglobulin variable region polypeptides, multispecific multimers can also be produced that comprise domains specific for more than two targets/epitopes, including trispecific and or multispecific formats, see for instance PCT/NL2019/050199. Although strategies exist in the art to increase the yield of the desired bispecific or multispecific proteins or antibodies, the production of undesired species including monospecific proteins or halfbodies cannot readily be entirely avoided. Hence, separation of the bispecific or multispecific proteins or antibodies from the monospecific, halfbodies or unwanted byproduct proteins is preferred to isolate the desired bispecific or multispecific proteins or antibodies. Such separation of these bispecific or multispecific proteins or antibodies further can be a requirement for clinical development or marketing such proteins.

The current inventors now surprisingly found that by producing immunoglobulin regions with charged residues at non-surface exposed amino acid positions within the constant region, preferably the CH1, CH2 or CH3, including residues that are buried within the immunoglobulin polypeptide, multispecific or bispecific proteins and monospecific proteins, when produced, can now readily be separated and obtained by using isoelectric focusing and conventional chromatography methods. e.g. non-affinity based chromatography such as ion-exchange chromatography.

Such immunoglobulin regions include the addition, removal or reversal of charge to one or both of the constant region-containing immunoglobulin polypeptide chains, preferably at the CH1, CH2, CH3 or combination thereof. Prior to the present invention, modification of non-surface exposed or buried amino acids of any protein, particularly immunoglobulins, has in general been avoided, as it has been understood that altering a charge of such residues has a potentially deleterious effect on structure and function, including the potential to cause a destabilizing impact on the immunoglobulin. Further, such modifications would not be expected to alter chromatographic properties as these residues are not readily exposed for interaction with chromatographic resins.

Surprisingly it was found that by producing immunoglobulin regions having charged amino acids at non-surface exposed, and buried amino positions, including within the framework or constant region, preferably the CH1, CH2, CH3 region or combination thereof of immunoglobulin polypeptide chains, monospecific, bispecific and multispecific proteins can be produced that have a differentiated charge, and different isoelectric points (See e.g. FIG. 1A-1C), which permit separation and isolation of said monospecific proteins from bispecific or multispecific proteins (or vice versa) or separation of desired proteins from other unwanted protein byproducts. Further, immunoglobulin regions may be produced comprising charged residues and other variations at non-surface exposed or buried positions, which may have potential to increase stability of such immunoglobulin regions over wild-type regions or domains or wild-type regions or domains having solely the charge variations.

It is understood that the variant domains, including constant domains, and methods of employing such domains, can be applied to produce multimerizing proteins, and to separate such proteins. When different protein species are produced in a mixture, such that said different species have similar isoelectric points (pI), making separation difficult, use of variant domains set out herein and the methods described herein can be employed to improve separation of the desired species.

The invention discloses methods to select variations that do not deleteriously affect structure and function of the separation domain and produce differentiated isoelectric points between different multimerized protein species produced incorporating such domains. An invention described herein applies to products comprising separation domains that may be applied to a variety of immunoglobulin regions, e.g. CL, CH1, CH2 and/or CH3 regions, and the VH/VL regions (in particular the framework regions). The invention may, in general, be applied to any multimer protein. As long as the multimer that is produced comprises at least two different proteins (e.g. denoted A and B), which can form different multimerizing proteins, e.g. such that multimeric species produced can comprise AA, AB, BA or BB, the invention can be applied thereto. In such circumstances, these multimerizing proteins may employ variant domains of the invention to chain A and/or chain B, such that each of the multimer species may comprise one or more variant domain having charges at non-surface exposed or buried positions, and produce multimer species that comprises differentiated isoelectric points to allow for separation via methods known to persons of ordinary skill in the art, such as by isoelectric focusing and/or based on distinctive retention times during charge chromatography.

The above principle can also be applied to making bispecific antibodies (yielding up to ten species when two different heavy chains and two different light chains are expressed or three species when two different heavy chains and a common light chain are expressed, or when two different light chains and a common heavy chain are expressed). The above principle can also be applied to higher multimers.

Where multimers may be bispecific antibodies, variant immunoglobulin regions having a change in charge(s) at non-surface or buried positions, including addition, reduction or reversal of charge may be employed. For example, charged CH1 regions may be employed (as exemplified in-FIGS. 1A-1C), or charged (CH2 region may be employed (as exemplified in FIG. 1D), charged CL region of the light chain may be employed (FIG. 1E), or charged CH3 regions may be employed.

Such multimers may also be tri- or quadrivalent, such that they may comprise e.g. 3 variable domains, consisting of a VH and a VL, comprising (as exemplified FIGS. 2A and 2B e.g. a variations in a CH1 region or a CL region.

It is understood that reference made herein to “variation” of an immunoglobulin region, such as a CH1 region, or any other suitable region or domain, does not imply that e.g. a multimer protein product such as an antibody is being mutated, but rather that the multimer protein comprises a domain having the separation variants set out herein, which differ, for example, from a wild-type domain. That is, such domains contain differences at non-surface exposed residues within a wild type domain, thereby producing a charge differential, which can be used to facilitate separation from mixtures of multimerizing proteins. The term variation, hence refers to the fact that the amino acid sequence of an immunoglobulin polypeptide, such as comprised in a bispecific antibody, has an amino acid sequence that is different, e.g. different from a reference sequence such as a human IgG1 sequence.

It is understood that amino acid sequences having the desired residues at the desired location may be selected from libraries comprising variations within the amino acid sequence as compared to a reference sequence, e.g. in the CH1, CH2, CH3 region or combination thereof. Hence, the term variation refers to an amino acid sequence having the desired amino acid residue at a desired location independent of the manner in which the amino acid sequence was obtained. Instead of referring to amino acid variations as described herein, e.g. of amino acids at non-surface amino acids, preferably buried amino acids, one may also refer to “separation amino acid residues”, as these variations allow for separation of desired multimeric species.

Protein products can be produced from DNA constructs encoding the proteins, hence, a variation of a protein product can have its origin in the DNA construct that encodes said protein. Any suitable means of generating such variations known in the art are encompassed herein, for example including constructs which can be generated comprising nucleic acid encoding such variations from the beginning, for example via DNA synthesis without employing any means of mutagenesis, replacement, substitution, insertion or deletion necessary to an original nucleic acid. Such manner of producing variation domains are ready for use and are capable to be combined with e.g. any suitable nucleic acid encoding any variable region (or any combination of CDR sequences comprised therein, should a modified variable region be used). Also, constructs can simply be synthesized de novo encoding a variable region of choice, combined with an encoded constant region having suitable amino acid variations as described herein that provide for differentiation in isoelectric points. For example, a CH1, CH2, CH3 encoding sequence can be provided and combined with a selected VH encoding sequence, this combination can be done in silico (and synthesized de noro) and/or in vitro (e.g. using molecular biology techniques such as ligation/cloning), and expression cassettes generated. By providing sequences of suitable combinations of variable regions (as encoded by a nucleic acid sequence), these can easily be combined with suitable constant regions. e.g. CL or CH1, and CH2 and/or CH3, in accordance with the invention encoding variations in accordance with the invention, e.g. having variations at non-surface amino acids, preferably buried amino acids, preferably within the CH1 region.

One can also provide a cell with suitable expression cassettes encoding for the components, e.g., polypeptides, that comprise the multimeric protein, such as e.g. a common light chain and two separate heavy chains. Said cell may from the beginning have stably integrated nucleic acid encoding suitable variant domains for separation. Such cells thereafter need only be integrated with a nucleic acid encoding a selected VL or VH region, or both (or replace VL and/or VH regions), which can then generate mixtures of multimeric proteins, which can be readily separated based on the variant domain(s). Accordingly, one aspect of the invention set out here comprises a host cell having stably integrated into its genome a nucleic acid encoding a common light chain, and a constant region comprising one or more domains comprising a separation amino acid residue set out herein. Preferably, said invention includes a nucleic acid encoding a domain comprising a negative separation amino acid residue for combination with a heavy chain variable region, and a domain comprising a positive separation amino acid residue for combination with a second heavy chain variable region. Preferably said two encoded heavy chain variable regions have different pI, wherein the more positive variable region may be linked to the domain comprising a positive separation amino acid residue and wherein the more negative variable region may be linked to the domain comprising a negative separation amino acid residue.

The invention also discloses a number of said variations or separation amino acid residues. Because these variations include non-surface residues of the proteins, this is also advantageous as these variations may reduce unwanted immunological effects because these variations may not result in exposure of potential antigenic motifs at the surface of the domain used for separation, such as a CH1 region in, for instance, a multispecific protein, in particular multispecific antibodies. Furthermore, because said variations are not at the surface of a protein, selected variations as disclosed herein can advantageously be applied in general to any bispecific or multispecific protein comprising a constant region or framework region comprising a variation as disclosed herein and a compatible heterodimerization region (for example a CL, CH2 or CH3 domain), preferably at least two constant region domains, more preferably CH1, comprising immunoglobulin polypeptides. Preferably, multispecific proteins in accordance with the invention having variable heavy chain domains and variable light chain domains may have one or more amino acid changes selected within the framework or constant region, preferably the CH1 region, that are non-surface exposed or buried that do not deleteriously impact the CH/CL interface for CH1, or the CH2-CH3/CH2-CH3 domain where residues are modified at the Fe interface.

Alternatively, a multispecific protein of the invention may comprise a separation domain, such as a CH1 region that does not need to pair with a CL. For example, the CH1 could be a camelid CH1 or based on a camelid CH1 or other organisms that lack a light chain such as sharks, or could be a modified CH1 region that lacks hydrophobic residues and does not pair with a light chain, wherein the domain includes a variation at a non-surfaced exposed residue to produce an isoelectric point differential to facilitate separation of the multispecific protein from other proteins, and fragments.

By including such variations in the CH1 region, said variations beneficially do not impact Fc/Fc receptor interactions or multimerization (e.g., hetero- or homodimerization) of the peptides, typically at the CH2-CH3/CH2-CH3 interface. Preferably, domains of the invention comprising one or more separation residues selected within the framework or constant region, preferably the CH1 region, that are non-surface exposed or buried, in addition to another variation that may beneficially improve stability compared to a wild-type domain or to a domain comprising one or more separation residues alone.

Accordingly, in one embodiment, a bispecific protein, in particular an antibody, is provided comprising a first CH1-containing immunoglobulin polypeptide and a second CH1-containing immunoglobulin polypeptide, wherein the first and/or second CH1-containing immunoglobulin polypeptides comprise one or more variant separation amino acid residues that are non-surface exposed or buried, such that the isoelectric point of the immunoglobulin protein comprising the first CH1-containing immunoglobulin polypeptide and the second CH1-containing immunoglobulin polypeptide is different from the isoelectric points of proteins having only the first CH1-containing immunoglobulin polypeptides and/or proteins having only the second CH1-containing immunoglobulin polypeptides (e.g. parent proteins).

In one embodiment, variations of a CH1 containing immunoglobulin increase or decrease the retention time of said immunoglobulin on ion exchange chromatography.

This applies as well to a multispecific antibody comprising an immunoglobulin polypeptide comprising a first and second CH1 region dimerizing with an immunoglobulin polypeptide comprising e.g. a third CH1-containing immunoglobulin polypeptide, wherein the first and second CH-containing immunoglobulin polypeptide comprise one or more variant separation residues of one or more amino acids selected from amino acids within the CH1 region that are non-surface exposed or buried, such that the isoelectric point of the immunoglobulin protein comprising the first and second CH1-containing immunoglobulin polypeptide and the third CH1-containing immunoglobulin polypeptide is different from the isoelectric points of proteins having only the first CH1 and second CH1-containing immunoglobulin polypeptide and/or proteins having only the third CH1-containing immunoglobulin polypeptides (e.g. parent proteins). See e.g. FIG. 2B.

It is understood that the bispecific proteins comprising the first and second CH1-containing immunoglobulin polypeptides can be difficult to separate from the parent proteins (e.g. monospecific bivalent antibodies), using a conventional chromatography method such as ion-exchange and the like where isoelectric points of the respective proteins are similar. As shown in the example section, the similarity in isoelectric points can manifest is similar retention time in a selected chromatography column. The similarity can also be determined using e.g. isoelectric focusing as shown in the examples. During the production of mixtures of antibodies or proteins containing immunoglobulin domains, retention times may be similar such that the peaks of respective proteins will overlap making separation difficult. It is also understood that the terms “first” and “second” as referred to with regard to the first and second CH1-containing immunoglobulin polypeptides do not imply any order or preference and merely serve to indicate that these chains are different.

It is understood that variations of the CH1 region in accordance with the invention, are to affect the isoelectric point of the bispecific antibody, include the addition, removal or reversal of a charge. The addition of charge to a CH-containing polypeptide or the like, can be at one or each of the CH1 region(s) for each immunoglobulin polypeptide produced. The addition of a charge can be obtained by various means. A neutral amino acid can be varied (typically at the encoding DNA level in an expression construct) and changed to either an amino acid with a negative or a positive charge, resulting in the addition of a negative and a positive charge, respectively. A positive amino acid can be varied into an amino acid with a neutral or a negative charge, resulting in the addition of a negative charge, with changing an amino acid from a positive to a negative charge resulting in a relatively larger change. Conversely, a negative amino acid can be varied into an amino acid with a neutral or a positive charge, resulting in the addition of a positive charge, with changing an amino acid from a negative to a positive charge resulting in a relatively larger change.

Hence, in a further embodiment, the immunoglobulin protein in accordance with the invention comprises one or more variations of one or more non-surface exposed or preferably buried amino acids selected from the group consisting of:
a neutral amino acid to a negatively charged amino acid;
a positively charged amino acid to a neutral amino acid;
a positively charged amino acid to a negatively charged amino acid.
neutral amino acid to a positively charged amino acid;
a negatively charged amino acid to a neutral amino acid; and
a negatively charged amino acid to a positively charged amino acid.

Amino acids that have a positive charge are Lysine (Lys, K), Arginine (Arg, R) and Histidine (His, H). Preferably, when an amino acid with a positive charge is to be included in a chain or varied from a parent domain, a Lysine is selected. Amino acids that have a negative charge are Glutamate (Glu, E) and Aspartate (Asp, D). The remaining amino acids from an isoelectric point perspective represent neutral amino acids. Preferably, in a further embodiment, the immunoglobulin protein in accordance with the invention comprises one or more variations of one or more non-surface exposed or preferably buried amino acids selected from the group consisting of:
a neutral amino acid to a negatively charged amino acid;
a positively charged amino acid to a neutral amino acid;
a neutral amino acid to a positively charged amino acid; and
a negatively charged amino acid to a neutral amino acid.

These variations may be preferred as conservative design alterations.

As exemplified in FIGS. 1A-E and 2A-2B, which schematically depicts monospecific, bispecific, and exemplary multispecific antibodies in accordance with the invention, either one or both of a CH1-containing immunoglobulin can be varied. It is understood that variations selected for one of the CH1 containing immunoglobulins, or the like, are preferably of the same type, i.e. when a positive charge is added to one chain, one or more variations are selected that add a positive charge to that chain (to have an additive effect). It is also understood that when one of the chains has an added positive charge, and the other chain is to include one or more variations as well, that the variation or variations selected for the other chain are preferably selected to comprise the addition of a negative charge because otherwise the effect on the isoelectric points of the different paired immunoglobulin proteins comprising the first and second CH1 containing immunoglobulin may typically be counteracted or even nullified.

Hence, in one embodiment, an immunoglobulin protein is provided comprising a first CH1-containing immunoglobulin polypeptide and a second CH1-containing immunoglobulin polypeptide, wherein the first and/or second CH1-containing immunoglobulin polypeptides comprise one or more variations of one or more amino acids selected from amino acids within the CH1 region that are non-surface exposed, wherein the first CH1-containing immunoglobulin polypeptide comprises variations selected from:
a neutral amino acid to a negatively charged amino acid;
a positively charged amino acid to a neutral amino acid; and
a positively charged amino acid to a negatively charged amino acid;
   and, wherein the second CH1-containing immunoglobulin polypeptide comprises variations selected from:
a neutral amino acid to a positively charged amino acid;
a negatively charged amino acid to a neutral amino acid; and
a negatively charged amino acid to a positively charged amino acid.

In another embodiment, an immunoglobulin protein is provided comprising a first CH1-containing immunoglobulin polypeptide and a second CH1-containing immunoglobulin polypeptide, wherein the first and/or second CH1-containing immunoglobulin polypeptides comprise one or more variations of one or more amino acids selected from amino acids within the CH1 region that are non-surface exposed, wherein the first CH1-containing immunoglobulin polypeptide comprises variations selected from:
a neutral amino acid to a negatively charged amino acid; and
a positively charged amino acid to a neutral amino acid;
   and, wherein the second CH1-containing immunoglobulin polypeptide comprises variations selected from:
a neutral amino acid to a positively charged amino acid; and
a negatively charged amino acid to a neutral amino acid.

In one embodiment the first CH1-containing immunoglobulin polypeptide and the second CH1-containing immunoglobulin polypeptide, when aligned with respect to amino acid sequence of the CH1 region preferably are substantially identical and preferably only differentiate with regard to the amino acid positions as defined herein. Preferably, amino acid positions that differ between the CH1 regions of the first and second CH1-containing polypeptide differ with regard to non-surface exposed amino acid positions. Said CH1 containing polypeptide preferably is a human IgG1 immunoglobulin CH1 region. An example of an amino acid sequence of a CH1 region that is suitable for generating or comparing against a separation domain comprising variant residues as described herein is depicted in FIG. 14A-14C.

In a further embodiment, the immunoglobulin protein in accordance with the invention, comprises further in the first CH1-containing immunoglobulin polypeptide and/or second CH1-containing immunoglobulin polypeptide stabilizing variations further selected from amino acids within the CH1 region. Further variations may be introduced that are to increase stability of the polypeptide and/or the bispecific or multispecific protein comprising the domain containing the separation residues described herein.

Preferably, a non-surface exposed or buried separation residue within an immunoglobulin polypeptide may result in relative increased stability in comparison to a reference domain, such as a wild-type domain.

As used herein, the term “non-surfaced exposed” means scoring of 50% or less in “Ratio (%)” in the program GETAREA 1.0 beta using default parameters, wherein greater than 50% Ratio (%) is scored as “Out” or “surface exposed” in this program. As used herein, the term “buried” means scoring 20% or less Ratio (%) in the program GETAREA 1.0 using default parameters, which is scored as “In” in this program. Negi et al., “Solvent Accessible Surface Areas. Atomic Solvation Energies, and Their Gradients for Macromolecules”, Last modified on Wed 17th April, 3:00 PM, 2015. The primary amino acid and a structural model of the protein domain containing the region are used as an input into the GETAREA program to obtain the “Ratio (%)” in the GETAREA output files, such as provided in the examples herein. Where herein reference is made to a buried amino acid reference is made to an amino acid or a variation thereof that has a scoring of 20% or less and preferably 15% or less Ratio (%) as indicated in table 1 and tables 20-22. In some embodiments the reference to a buried amino acid refers to an amino acid or a variation thereof that has a scoring of 10% or less Ratio (%) as indicated in table 1 and tables 20-22.

Structural information for the CH region can be obtained from the Protein Data Bank, which contains several high resolution structures for each of the CH regions, or via homology modelling (e.g. using a homology modelling tool to account for modelling the structure of CH regions that contain variations; https://swissmodel.expasy.org). Structural information of a selected CH1 region, or the like, as provided in a pdb format is imported in the Getarea program (Protein Data Bank format, providing a standard representation for macromolecular structure data derived from X-ray diffraction and NMR studies), which registers the Ratio (%) scoring upon submission for analysis.

As used herein, “pI” is calculated based on the primary amino acid according to ExPASy, ProtParam tool, using default parameters. The ProtParam is a tool which allows the computation of various physical and chemical parameters for a given protein stored in Swiss-Prot or TrEMI3L or for a user entered protein sequence. The computed parameters include the theoretical pI. Gasteiger E., Hoogland C., Gattiker A., Duvaud S., Wilkins M. R., Appel R. D., Bairoch A.: Protein Identification and Analysis Tools on the ExPASy Server; (In) John M. Walker (ed): The Proteomics Protocols Handbook, Humana Press (2005) pp. 571-607. The full polypeptide is used to measure theoretical pI, such as provided in the examples herein.

As shown in the example section, a further selection can be made for each amino acid position along a domain of interest, e.g. by performing an in silico stability analysis, such as relying on Rosetta software (version 3.1 <<https://www.rosettacommons.org/software>>) of non-surface exposed and buried residues without altering surface exposed residues. Instead of a selection in silico, this can also be carried out in vitro. In addition, the selection can in first, instance be in silico followed subsequently by confirmation thereof in vitro, such as shown in the example section.

An “antibody” is a proteinaceous molecule belonging to the immunoglobulin class of proteins, containing one or more domains that bind an epitope on an antigen, where such domains are derived from or share sequence homology with the variable region of an antibody. Antibody binding has different qualities including specificity and affinity. The specificity determines which antigen or epitope thereof is specifically bound by the binding domain. The affinity is a measure for the strength of binding to a particular antigen or epitope. It is convenient to note here that the ‘specificity’ of an antibody refers to its selectivity for a particular antigen, whereas ‘affinity’ refers to the strength of the interaction between the antibody's antigen binding site and the epitope it binds.

Antibodies for therapeutic use are preferably as close to natural antibodies of the subject to be treated as possible (for instance human antibodies for human subjects). An antibody according to the present invention is not limited to any particular format or method of producing it.

A “bispecific antibody” is an antibody as described herein wherein one domain of the antibody binds to a first antigen or epitope whereas a second domain of the antibody binds to a second antigen or epitope, wherein said first and second antigens are not identical or the first and second epitopes are not identical. The term “bispecific antibody” also encompasses antibodies wherein one heavy chain variable region/light chain variable region (VH/VL) combination binds a first epitope on an antigen and a second VH/VL combination that binds a second epitope. The term further includes antibodies wherein a VH is capable of specifically recognizing a first antigen and the VL, paired with the VH in an immunoglobulin variable region, is capable of specifically recognizing a second antigen. The resulting VI/VL pair will bind either antigen 1 or antigen 2. Such so called “two-in-one antibodies”, described in for instance WO 2008/027236, WO 2010/108127 and Schaefer et al (Cancer Cell 20.472-486. October 2011). A bispecific antibody according to the present invention is not limited to any particular bispecific format or method of producing it. A bispecific antibody is a multispecific antibody. Multispecific multimers or antibodies as referred to herein, encompass proteinaceous molecules belonging to the immunoglobulin class of proteins, containing two or more domains that bind an epitope on an antigen, where such domains are derived from or share sequence homology with the variable region of an antibody, and include proteinaceous molecules binding three antigens or more as known in the art, including as described in the previously filed application U.S. 62/650,467.

A domain of an invention described herein comprises a framework or constant domain that differs from a wildtype or reference sequence, such that it comprises a negatively charged amino acid, wherein the corresponding position of the wildtype or reference sequence is non-surface exposed or buried, and contains a neutral amino acid. Alternatively, a domain of an invention described herein comprises a framework or constant domain that differs from a wildtype or reference sequence, such that it comprises a positively charged amino acid, wherein the corresponding position of the wildtype or reference sequence is non-surface exposed or buried, and contains a neutral amino acid. Alternatively, a domain of an invention described herein comprises a framework or constant domain that differs from a wildtype or reference sequence, such that it comprises a neutral amino acid, wherein the corresponding position of the wildtype or reference sequence is non-surface exposed or buried, and contains a positive or negative amino acid. Alternatively, a domain of an invention described herein, comprises a combination of embodiments described above, such that the net pI of the domain is different by one or more charges from the wildtype or reference sequence.

The term ‘charged amino acid residue’ or ‘charged residue’ as used herein means amino acid residues with electrically charged side chains at physiological relevant pH. These may be either be positively charged side chains, such as present in arginine (Arg, R), histidine (His, I) and lysine (Lys, K) or can be negatively charged side chains, such as present in aspartic acid (Asp, D) and glutamic acid (Glu, E). The term ‘neutral amino acid residue’ or neutral residue as used herein refers to all other amino acids that do not carry electrically charged side chains at physiologically relevant pH. These neutral residues include serine (Ser, S), threonine (Thr, T), asparagine (Asn, N), glutamine (Glu, Q), Cysteine (Cys, C), glycine (Gly, G), proline (Pro, P), alanine (Ala, A), valine (Val, V), isoleucine (Ile, I), leucine (Leu, L), methionine (Met, M), phenylalanine (Phe, F), tyrosine (Tyr, Y), and tryptophan (Trp, T).

A preferred embodiment of an invention described herein, comprises a separation domain as described above, and/or a protein comprising such a separation domain. A separation domain of an invention described herein may be incorporated into an antibody or a protein having an immunoglobulin domain. It may be incorporated into IgG of any subclass or T-cell receptor domain or immunoglobulins that are mono- or multispecific.

A further preferred embodiment of an invention described herein, is a protein comprising one or more binding domains and comprises a CH1 separation domain comprising an N159K, H or R or N159D or E separation residue, and more preferably a N159K or N159D separation residue. A further preferred embodiment of an invention described herein, is a protein comprising one or more binding domains and comprises a CH1 separation domain comprising an N201K, H or R or N201D or E separation residue, and more preferably a N201K or N201D separation residue.

A monospecific, bispecific or multispecific protein as provided in accordance with the invention, incorporating a separation domain of an invention set out herein, may comprise a CH1 region selected to be a CH1 region from a human IgG, which in one embodiment comprises amino acids within the CH1 region that are selected from the group comprising of T120, K147, D148, Y149, V154, N159, A172, Q175, S190, N201, and K213. The numbering of these amino acid positions is in accordance with EU-numbering.

A CH1 separation domain of an invention disclosed herein may further comprise a stabilizing variation corresponding to T197D.

A CH1 separation domain of an invention disclosed herein may further comprise a stabilizing variation corresponding to the hinge at E216K

A CH1 separation domain of an invention disclosed herein may further comprise a stabilizing variation corresponding to G122P, S157T, I199V, N203, S207T, and V211I.

By producing and employing separation domains comprising variations as disclosed herein, e.g., via variation residues amino acids selected from the group comprising of T120, K147, D148, Y149, V154, N159, A172, Q175, S190, N201, and K213 within the CH1 region of human IgG1 immunoglobulin polypeptide chains, monospecific proteins, bispecific proteins or multispecific proteins can be produced that have a differentiated charge at pH used during formulation and separation. i.e. different isoelectric points (See e.g. FIG. 1A-1E), which allows for separation and isolation of monospecific proteins from bispecific or multispecific proteins (or bispecific proteins from trispecific, and so on).

In one embodiment, a monospecific, bispecific or multispecific protein is produced in accordance with the invention wherein the CH1 region of the immunoglobulin polypeptide comprises a separation residue at the CH1 region that is a non-surface exposed or buried amino acid, selected from the group consisting of D148, Y149, V154, N159, A172, S190 and N201. Said protein is preferably a human protein, preferably an IgG protein, preferably an IgG1 protein.

In one embodiment, for a bispecific protein in accordance with the invention wherein the CH1 region of the immunoglobulin polypeptide is selected to be a CH1 region from a human IgG1, the amino acids within the CH1 region selected from the group consisting of T120, K147, D148, N159, Q175, N201, K213, as these amino acid positions allow for a variations having a different charge (changing between a neutral, positively and negatively charged amino acid). In a further embodiment, the amino acids within the CH region that are non-surface exposed amino acids are selected from the group consisting of amino acids N159 and N201, which are buried amino acids. More preferably said immunoglobulin protein is a bispecific antibody or multispecific protein.

Most preferably, said first and second CH1-containing immunoglobulin polypeptides each comprise a heavy chain variable region, wherein each of said variable region binds to a different antigen or epitope.

In another embodiment, a bispecific protein is provided in accordance with the invention comprising a first CH1-containing immunoglobulin polypeptide and a second CH1-containing immunoglobulin polypeptide, said CH1 region being a human IgG1 CH1 region, wherein the first or second CH1-containing immunoglobulin polypeptides comprise one or more variations of amino acids selected from amino acids within the CH1 region, said variations comprising one or more variations selected from the group consisting of K147E, N159D, Q175E, N201D, and K213Q, or one or more variations selected from the group consisting of T120K, D148K, N159K, Q175K, N201K. Most preferably said bispecific protein is a bispecific antibody.

In a preferred embodiment, the invention provides an immunoglobulin protein comprising a first CH1-containing immunoglobulin polypeptide and a second CH1-containing immunoglobulin polypeptide, said CH1 region being a human IgG1 CH1 region, wherein one of the first or second CH1-containing immunoglobulin polypeptides comprises variations N159K and at a hinge position at E216K. In a further embodiment, the other of the first or second CH1-containing immunoglobulin polypeptides comprises no variations or e.g. one or more variations such as e.g. selected from T197D and K213Q. Preferably a multimerizing protein of the invention is comprised of two polypeptides, wherein a first polypeptide comprises a first variable domain binding a first antigen or epitope and a second polypeptide comprises a second variable domain binding a different antigen or epitope than the first variable domain, wherein the first variable domain is linked via a peptide bond to a separation domain, which is linked to a dimerization domain, such as CH3, wherein said dimerization domain forms an interface with a second dimerization domain, such as a second CH3 domain, which is linked via a peptide bond to said second variable domain, and preferentially a second separation domain having a different charge than the first separation domain, wherein said protein preferably comprises a bispecific or multispecific protein or antibody.

In one embodiment, a monospecific, bispecific or multispecific protein is produced in accordance with the invention wherein the CH1, CH2, CH3 region or combination thereof of the immunoglobulin polypeptide comprises a separation residue at the CH region that is a non-surface exposed or buried amino acid, selected from the group consisting of T120, K147, D148, Y149, V154, N159, A172, Q175, S190, N201, and K213, V303, K370, EE382 and E388. Said protein is preferably a human protein, preferably an IgG protein, preferably an IgG1 protein.

In one embodiment, for a bispecific protein in accordance with the invention wherein the CH region of the immunoglobulin polypeptide is selected to be a CH region from a human IgG1, the amino acids within the CH region selected from the group consisting of T120, K147, D148, N159, Q175, N201, K213, V303, K370, E382 and E388 as these amino acid positions allow for a variations having a different charge (changing between a neutral, positively and negatively charged amino acid). In a further embodiment, the amino acids within the CH region that are non-surface exposed amino are selected from the group consisting of amino acids N159 and N201 for CH1, V303 for CH2 and E382 and E388 for CH3, which are buried amino acids. More preferably said immunoglobulin protein is a bispecific antibody or multispecific protein. Most preferably, said first and second CH-containing immunoglobulin polypeptides each comprise a heavy chain variable region, wherein each of said variable region binds to a different antigen or epitope.

In another embodiment, a bispecific protein is provided in accordance with the invention comprising a first CH-containing immunoglobulin polypeptide and a second CH-containing immunoglobulin polypeptide, said CH region being a human IgG1 CH region, wherein the first or second CH-containing immunoglobulin polypeptides comprise one or more variations of amino acids selected from amino acids within the CH1 region, said variations comprising one or more variations selected from the group consisting of K147E, N159D, Q175E, N201D, K213Q and V303E or one or more variations selected from the group consisting of T120K, D148K, N159K, Q175K, N201K, V303K, E382Q, E382T, E388L, E388M, E388T. Most preferably said bispecific protein is a bispecific antibody.

It is known in the art, that bispecific or multispecific antibodies may be preferentially produced over a monospecific antibody (or unwanted protein by-products) by having a bispecific or multispecific antibody, which comprises a first polypeptide comprising a CH3 region comprising variations L351D and L368E (“DE arm”), and a second polypeptide comprising a second CH3 region comprising variations T366K and L351K (“KK arm) (collectively referred to as a “DEKK” heterodimer) (EU-numbering), such that the two polypeptides forming the DEKK preferentially pair over two polypeptides comprising the either a DE/DE homodimer or KK/KK homodimer. Other forms of charge engineering are known in the art to promote heterodimerization.

In an embodiment described herein, where a negative separation domain, such as a CH1 region is used, it is preferentially paired with a DE CH3 domain, and where a positive separation domain, such as a CH1 region is used, it is preferentially paired with a KK arm or any combination of the forgoing (e.g., a negative separation domain and DE CH3 domain on one polypeptide and a positive separation domain and a KK CH3 domain on the other polypeptide to preferentially form a heterodimer that may be more readily separated from either a dual positive separation domain and KK/KK homodimer or a dual negative separation domain and DE/DE homodimer). To the extent other heterodimerization technology is employed, as known to a person of ordinary skill in the art, the invention applies in the same manner, by combining a negatively charged separation domain to a negatively charged heterodimerization domain and/or a positively charged separation domain to a positively charged heterodimerization domain to facilitate heterodimer formation and separation of said heterodimer.

In a further embodiment, a multimerizing protein, preferably a bispecific or multispecific protein is provided in accordance with the invention comprising a first CH1-containing immunoglobulin polypeptide and a second CH1-containing immunoglobulin polypeptide, said CH1 region being a human IgG1 CH1 region, wherein the first CH1-containing immunoglobulin polypeptide comprises one or more variations of amino acids selected from amino acids within the CH1 region, said variations comprising one or more variations selected from the group consisting of K147E, N159D, Q175E, N201D, and K213Q, and wherein the second CH1-containing immunoglobulin polypeptide comprises one or more variations of amino acids selected from amino acids within the CH1 region, said variations comprising one or more variations selected from the group consisting of T120K, D148K, N159K, Q175K, and N201K. Most preferably said bispecific protein is a bispecific antibody.

In another embodiment, a multimerizing protein, preferably a bispecific or multispecific protein is provided in accordance with the invention comprising a first CH1-containing immunoglobulin polypeptide and a second CH1-containing immunoglobulin polypeptide, said CH region being a human IgG1 CH1 region, wherein the first or second CH1-containing immunoglobulin polypeptide comprises one or more variations of amino acids selected from amino acids within the CH1 region, said variations selected from the group consisting of K147E and Q175E; N201D and K213Q; T197D and K213Q; N159D and K213Q; and K213Q, or said variations selected from the group consisting of T120K; N201K; D148K and Q175K; and N159K and a variation of an amino acid at a hinge residue E216K. Most preferably said protein is a bispecific antibody.

In still a further embodiment, a multimerizing protein, preferably a bispecific or multispecific protein is provided in accordance with the invention comprising a first CH1-containing immunoglobulin polypeptide and a second CH1-containing immunoglobulin polypeptide, said CH region being a human IgG1 CH1 region, wherein the first CH1-containing immunoglobulin polypeptide comprises one or more variations of amino acids selected from amino acids within the CH1 region, said variations selected from the group consisting of K147E and Q175E; N201D and K213Q; T197D and K213Q; N159D and K213Q; and K213Q and wherein the second CH1-containing immunoglobulin polypeptide comprises one or more variations of amino acids selected from amino acids within the CH1 region, said variations selected from the group consisting of T120K; N201K; D148K and Q175K; and N159K and a variation of an amino acid at a hinge residue E216K. Most preferably said immunoglobulin protein is a bispecific antibody.

In one embodiment a multimerizing protein, preferably a bispecific or multispecific protein is provided in accordance with the invention comprising a CH region with a sequence of a CH1, CH2 or CH3 region as depicted in table 14 part B. The multimerizing protein, preferably a bispecific or multispecific protein may have CH1, CH2 or CH3 region that are a combination of two or three CH region sequences of table 14 part B. Where it has two CH1, two CH2 or two CH3 sequences as depicted in table 14 part B it is preferred that the two have opposite charge differences when compared to a wild type CH region. So if one has a more positive charge when compared to the wild type CH, the other preferably has a more negative charge when compared to the wild type CH. Heavy chains can have two or three sequences of table 14 by having for instance two or three of a CH1 sequence, a CH2 sequence and CH3 sequence of table 14 part B. In such a case the two or three may have the same charge difference when compared to a wild of the CH. The two or three all a more positive charge when compared to the wild types or the two or three all a more negative charge. Again the multimerizing protein preferably a bispecific or multispecific protein may have two of such heavy chains, in such cases it is preferred that the two have opposite charge differences when compared to a wild type heavy chain.

The multimerizing protein preferably a bispecific or multispecific protein is preferably a multispecific antibody, preferably a bispecific antibody.

Various approaches are described in the art in order to promote the formation of a multispecific protein of interest, such as a bispecific antibody, thereby reducing the content of a monospecific, bivalent (parent). For antibodies, the CH3-CH3 interaction is a driver for Fe dimerization. Variations of amino acids of the CH3 regions at the interface between two CH3 regions can be introduced to promote bispecific formation and/or disrupt monospecific parent formation (e.g. via introduction of compatible/repulsive charges or steric (in)compatibility). Such approaches can advantageously be combined with the variations of the CH region as described herein.

Accordingly, in a further embodiment, an immunoglobulin protein in accordance with the invention is provided, wherein the first and second CH1 containing immunoglobulin polypeptides comprise a CH3 region, and wherein one of the first and second CH1-containing immunoglobulin polypeptide comprises (CH3 variations L351D and L368E, and the other comprises CH3 variations T366K and L351K (also referred to as “DEKK”) (EU-numbering).

It is understood that as these so-called DEKK variations are in alignment with the addition of charge to the first and/or second CH1-containing immunoglobulin polypeptide. This means that when a CH-containing immunoglobulin polypeptide comprises variations that have added a negative charge, that chain will preferably have the L351D and L368E CH3 residues, the other chain, which need not be (but may be) a variant CH1 region, will have the CH3 T366K and L351K residues. Conversely, this means that when a CH1-containing immunoglobulin polypeptide comprises variations that have added a positive charge, that chain will preferably have the T366K and L351K CH3 variations, the other chain, which need not be a variant CH1 region, will have the L351D and L368E CH3 residues. Preferably, said immunoglobulin protein in accordance with the invention comprises a human immunoglobulin Fc region, most preferably said human immunoglobulin Fc region is an IgG1 Fc region. As noted above, where other charge variation CH3 technology may be employed for heterodimer formation, a preferred embodiment of a multimerizing protein of the invention comprises a separation domain-containing immunoglobulin polypeptide having a negative charge further comprising a multimerizing domain, such as a CH3 that has a negative charge, and a separation domain-containing immunoglobulin polypeptide having a positive charge further comprising a multimerizing domain, such as a CH3 that has a positive charge, to facilitate heterodimerization and separation of said multimerizing protein.

The terms ‘CH1 region’, ‘CH2 region’ and ‘CH3 region’ are well known in the art. The IgG structure has four chains, two light and two heavy chains; each light chain typically has two domains, the variable and the constant light chain (VL and CL) and each heavy chain typically has four domains, the variable heavy chain (VH) and three constant heavy chain domains (CH1, CH2, CH3). The CH2 and CH3 region of the heavy chain is called Fc (Fragment crystallizable) portion, Fc fragment, Fc backbone or simply Fc. The IgG molecule is a heterotetramer having two heavy chains that are held together by disulfide bonds (—S—S—) at the hinge region and between the CH1 and CL. The heavy chain dimerization includes interactions comprised at the CH3-CH3 domain interface and through interactions at the hinge region. Examples of amino acid sequences of suitable CH2, CH3 and hinge regions are depicted in FIG. 14A-14E.

In one embodiment, the immunoglobulin protein in accordance with an invention described herein, comprises a first CH1-containing polypeptide which is an antibody heavy chain. In one embodiment, the immunoglobulin protein in accordance with an invention described herein comprises a second CH1-containing polypeptide which is an antibody heavy chain. In a further and preferred embodiment, both the first and second CH1 containing polypeptides are antibody heavy chains, such as human IgG1 heavy chains. Said immunoglobulin proteins in accordance with the invention can further comprise one or more antibody light chains. Most preferably said antibody light chain is a common light chain.

The term ‘common light chain’ as used herein thus refers to light chains which may be identical or have some amino acid sequence differences while retaining the binding specificity of the resulting antibody after pairing with a heavy chain. It is for instance possible to prepare or find light chains that are not identical in amino acid sequence but still functionally equivalent. e.g. by introducing and testing conservative amino acid changes, and/or changes of amino acids in regions that do not or only partly contribute to binding specificity when paired with the heavy chain, and the like. A combination of a certain common light chain and such functionally equivalent variants is encompassed within the term “common light chain”. Reference is made to WO 2004/009618 for a detailed description of the use of common light chains Preferably, a common light chain is used in the present invention which is a germline-like light chain, more preferably a germline light chain, preferably a rearranged germline human kappa light chain, most preferably either the rearranged germline human kappa light chain IgVκ1-39Jκ or IGVκ3-20/Jκ. Other light chains encompassed within the inventions disclosed herein include IgKV3-15/JK1 and surrogate light chains, which are also known in the art to constitute common light chains.

As an alternative to using a common light chain and to avoid mispairing of unmatched heavy and light chains, means for forced pairing of the heavy and light chain, such as for example described in WO2009/080251, WO2009/080252 and/or WO2009/080253 may be contemplated. Examples of amino acid sequences of common light chain variable regions, common light chains and/or CDR sequences for a common light chain are depicted in FIG. 13A-13P. A preferred common light chain has a sequence as depicted in FIG. 13A-13E.

As the variant residues of the CH1 region are selected among those amino acids within the CH1 region that are non-surface exposed amino acids, or preferably buried amino acids as described above, such variations are in particular suitable for human bispecific proteins, as these variations allow for a bispecific protein that most closely resembles the tertiary structure of human antibodies. It is understood that of the term human in reference to protein description does not imply that the entire amino acid sequences of the first and second CH1-containing polypeptides needs to be of human origin, nor that the amino acid sequences need to be directly obtained from a human. It is understood that reference to a human domain, protein or antibody refers to a protein that may include some alterations of the amino acid sequences. e.g. CH2 engineering (including for Fc silencing) CH3 engineering (including for heterodimerzation) and/or Fe engineering (including for impacting Fe receptor activity), and for inclusion of separation residues. The human domains used to generate the bispecific or multispecific proteins may be encoded by nucleic acid sequences obtained from mice harboring features of a human immune system, such as a heavy, light or hybrid loci encoding human variable region gene segments and/or constant regions, as known in the art. WO2009/157771. Such proteins may also be obtained through identification of nucleic acid encoding human immunoglobulin domains identified from phage display, yeast display, and other techniques well known to those of ordinary skill in the art.

Multimerizing proteins in accordance with the invention can be bispecific or multispecific proteins, preferably antibodies which, although not occurring in nature, can also be of human sequence, as the sequences of e.g. the two heavy chains and the two (common) light chains that combine into a human bispecific antibody, which may have minor variations from an amino acid sequence perspective as e.g. described herein, including variations of the CH1 region and/or preferably CH3 engineering and the like.

In one embodiment, the immunoglobulin protein in accordance with the invention, further comprising light chains, preferably has one or more variations of one or more amino acids within the CH1 region that are non-surface exposed and that are located distant from the CH1/CL interface. This way, any potential effects on the functionality of an antigen binding domain, including pairing of heavy and light chains can be avoided. Preferably, the bispecific protein in accordance with the invention is a bispecific antibody. More preferably, said bispecific antibody is a human bispecific antibody. Most preferably, said bispecific antibody of a human bispecific antibody is an IgG1 antibody.

In one embodiment, a nucleic acid encoding a separation domain of the invention, such as an immunoglobulin CH1-containing polypeptide comprising one or more variations selected from amino acids within the CH1 region that are non-surface exposed, is provided. Furthermore, another embodiment of an invention described herein is a cell or a recombinant host cell comprising nucleic acid encoding a separation domain of the invention. Further, in another embodiment, a cell or recombinant host cell comprising one or more nucleic acids encoding a first and second CH1-containing immunoglobulin polypeptides in accordance with the invention is provided. Such isolated nucleic acids, cells and recombinant host cells being in particular suitable for the production of the immunoglobulin proteins in accordance with an invention disclosed herein, and suitable for methods of separation of such immunoglobulin proteins.

Also provided are host animals or transgenic animals, comprising nucleic acids encoding variant separation domains as disclosed herein in accordance with the invention. Said host animals or transgenic animals in one embodiment encode an immunoglobulin region comprising one or more separation residues that correspond to non-surface exposed amino acid residues of wild-type immunoglobulin domains in accordance with the invention. Preferably such a transgenic animal is a rodent or bird, more preferably a mouse, rat, or chicken wherein at least part of the antibody repertoire of said mouse, rat, or chicken is human or humanized.

Hence, in one embodiment, a composition comprising an immunoglobulin protein in accordance with the invention as described herein is provided. It is understood that such a composition may be an intermediate product, e.g. a crude cell lysate and/or filtered crude lysate or semi-purified product. When such a composition is further processed, e.g. including separation steps that allow obtaining the bispecific protein due to the variations of the CH1 region(s) in accordance with the invention. A pharmaceutical composition may be obtained, i.e. comprising the bispecific protein in accordance with the invention and comprising pharmaceutically acceptable excipients. Such a product may be in the form of a liquid or in the form of a freeze dried product. Any pharmaceutically acceptable composition may be employed. It is understood that such a pharmaceutically acceptable composition may not be necessarily administered directly to a patient, but may be subjected to further preparatory steps, e.g. dissolving or mixing the pharmaceutical product in an appropriate solution for infusion to a patient. The above applies further to compositions comprising trispecific proteins and other multispecific proteins, comprising separation domains other than a CH1 region as further described throughout.

Further embodiments as described below relate to methods for producing immunoglobulin proteins in accordance with the invention.

In one embodiment, a method is provided for producing a variant bispecific protein in accordance with the invention, comprising the steps of:

a) providing a nucleic acid encoding a first CH-containing immunoglobulin polypeptide and a nucleic acid encoding a second CH-containing immunoglobulin polypeptide, said first and second CH-containing immunoglobulin polypeptides encoding a bispecific protein;
b) said nucleic acid encoding the first CH-containing immunoglobulin polypeptide comprising one or more variations of triplets encoding one or more amino acids within the CH region that are non-surface exposed, such that the isoelectric point of the variant bispecific protein comprising the first CH-containing immunoglobulin polypeptide and the second CH-containing immunoglobulin polypeptide is different from the isoelectric points of the monospecific proteins containing only the first CH-containing immunoglobulin polypeptide or only the second CH-containing immunoglobulin polypeptide:
c) providing a cell with the nucleic acid encoding the first CH-containing immunoglobulin polypeptide and the nucleic acid encoding the second CH-containing immunoglobulin polypeptide and producing the variant bispecific protein.

As described already above, it is understood that any variation may be performed in silico in steps a) and b) described above and below. Hence, the first and second CH-containing immunoglobulin polypeptides may be varied entirely in silico as compared to a reference sequence. It is understood that said variation may also comprise simply providing said (variant) nucleic sequences and ligating these e.g. suitable variable domains. Any way of construction, including standard molecular techniques, DNA synthesis and/or in silico design may be employed in accordance with the invention and be used in steps a) and b) as described above and below. It is also understood that the provision of nucleic acid to a cell may include any suitable method, such as transient and stable transfections or the like. It is also understood that the step of providing a cell with the nucleic acid of step c) may also include providing only a part thereof, as long as the end result is that cell is provided with the nucleic acid encoding the first CH-containing immunoglobulin polypeptide and the nucleic acid encoding the second CH-containing immunoglobulin polypeptide and said cell is capable of producing the variant bispecific protein. In another embodiment, a method for producing a variant bispecific protein in accordance with the invention is provided, wherein the method comprises the steps of:

a) providing a nucleic acid encoding a first CH-containing immunoglobulin polypeptide and a nucleic acid encoding a second CH-containing immunoglobulin polypeptide, said first and second CH-containing immunoglobulin polypeptides encoding a bispecific protein;
b) wherein the nucleic acid encoding the first CH-containing immunoglobulin polypeptide and the nucleic acid encoding the second CH-containing immunoglobulin polypeptide comprise one or more variations of triplets encoding one or more amino acids within the CH regions that are non-surface exposed, such that the isoelectric point of the variant bispecific protein comprising the first CH-containing immunoglobulin polypeptide and the second CH-containing immunoglobulin polypeptide is different from the isoelectric points of the parent proteins containing only the first CH-containing immunoglobulin polypeptide or only the second C-containing immunoglobulin polypeptide;
c) providing a cell with the nucleic acids encoding the first and modified second CH-containing immunoglobulin polypeptides and producing the variant immunoglobulin bispecific protein.

Preferably, said methods in accordance with the invention comprising one or more of said variations are selected from:
changing an amino acid from a neutral amino acid to a negatively charged amino acid
changing a positively charged amino acid to a neutral amino acid;
changing a positively charged amino acid to a negatively charged amino acid.
changing an amino acid from a neutral amino acid to a positively charged amino acid;
changing a negatively charged amino acid to a neutral amino acid; and
changing a negatively charged amino acid to a positively charged amino acid.

It is understood that preferably one of the CH-containing immunoglobulin polypeptides will have an added positive charge, or have an added negative charge. Both CH-containing immunoglobulin polypeptides may have an added charge, wherein preferably one CH-containing immunoglobulin polypeptide will have an added negative charge and the other CH-containing immunoglobulin polypeptide will have an added positive charge.

Hence, in a further embodiment, a method for producing a bispecific protein in accordance with the invention is provided comprising a first CH-containing immunoglobulin polypeptide and a second CH-containing immunoglobulin polypeptide, wherein the method comprises the steps of

a) providing a nucleic acid encoding a first CI-containing immunoglobulin polypeptide and a nucleic acid encoding a second CH-containing immunoglobulin polypeptide; wherein the first and/or second CH-containing immunoglobulin polypeptides comprise one or more variations of one or more amino acids selected from amino acids within the CH region that are non-surface exposed, wherein the first CH-containing immunoglobulin polypeptide comprises variations selected from:
a neutral amino acid to a negatively charged amino acid;
a positively charged amino acid to a neutral amino acid; and
a positively charged amino acid to a negatively charged amino acid; and wherein the second CH-containing immunoglobulin polypeptide comprises variations selected from:
a neutral amino acid to a positively charged amino acid;
a negatively charged amino acid to a neutral amino acid; and
a negatively charged amino acid to a positively charged amino acid;
   b) providing a cell with the nucleic acid encoding the first and second CH-containing immunoglobulin polypeptide and producing the bispecific protein.

It is understood that any variation steps may be performed in silico in steps a). Hence, the first and second CH-containing immunoglobulin polypeptides may be varied entirely in silico as compared to a reference sequence. It is understood that said variation may also comprise simply providing said nucleic sequences and ligating these e.g. suitable variable domains. Any way of construction, including standard molecular techniques, DNA synthesis and/or in silico design may be employed in accordance with the invention and be used in step a). It is also understood that the provision to a cell may include any suitable method, such as transient and stable transfections or the like. It is also understood that the step of providing a cell with the nucleic acid of step b) may also include providing only a part thereof, as long as the end result is that cell is provided with the nucleic acid encoding the first CH-containing immunoglobulin polypeptide and the nucleic acid encoding the second CH-containing immunoglobulin polypeptide and said cell is capable of producing the variant bispecific protein.

In a further embodiment, the step of varying the amino acid sequence of a CH-containing immunoglobulin polypeptide includes in addition the introduction of stabilizing modifications at further amino acid positions corresponding with one or more amino acids within the CH region.

The above applies further to methods of producing trispecific proteins and other multispecific proteins, nucleic acids that encode such proteins, and that comprise and encode separation domains other than a CH1 region having variations at non-surface exposed residues.

According to the invention a cell is provided comprising nucleic acid encoding at least a first and a second CH-domain comprising polypeptide chain, in accordance with the invention. Said cell according to the invention can further comprise a nucleic acid encoding a light chain, preferably a common light chain. Any cell for manufacturing immunoglobulin proteins in accordance with the invention may be employed, which includes any cell capable of expressing recombinant DNA molecules, including bacteria such as for instance Escherichia (e.g. E. coli), Enterobacter, Salmonella, Bacillus, Pseudomonas, Streptomyces, yeasts such as S. cerevisiae, K. lactis, P. pastoris, Candida, or Yarrowia, filamentous fungi such as Neurospora, Aspergillus oryzae, Aspergillus nidulans and Aspergillus niger, insect cells such as Spodoptera frugiperda SF-9 or SF-21 cells, and preferably mammalian cells such as Chinese hamster ovary (CHO) cells, BHK cells, mouse cells including SP2/0 cells and NS-0 myeloma cells, primate cells such as COS and Vero cells, MDCK cells, BRL 3A cells, hybridomas, tumor-cells, immortalized primary cells, human cells such as W138, HepG2, HeLa, HEK293, HT1080 or embryonic retina cells such as PER, C6, and the like.

Often, the expression system of choice will involve a mammalian cell expression vector and host so that proteins are appropriately glycosylated. A human cell line can be used to obtain bispecific antibodies with a completely human glycosylation pattern. In general, principles, protocols, and practical techniques for maximizing the productivity of mammalian cell cultures can be found in Mammalian Cell Biotechnology: a Practical Approach (M. Butler, ed., IRL Press, 1991). Expression of antibodies in cells and in recombinant host cells has been extensively described in the art. Hence, nucleic acids encoding the proteins of the invention comprise all elements that allow expression of the components of the bispecific proteins (e.g. two heavy chains and a light chains), such as e.g. promoter sequences, 5′/3′ UTRs, intron sequences, and the like. The nucleic acids encoding protein in accordance with the invention may be present as extrachromosomal (stably) transfected copies and/or stably integrated into a chromosome of the host cell. The latter is preferred.

Immunoglobulin polypeptides of the invention are expressed in host cells and are harvested from the cells or, preferably, from the cell culture medium by methods that are generally known to the person skilled in the art. After harvesting, the immunoglobulin protein comprising the first and second CH-containing immunoglobulin peptides (or the like) may be purified by using conventional methods known in the art. Such methods may include precipitation, centrifugation, filtration, size-exclusion chromatography, affinity chromatography. For a mixture of antibodies comprising IgG polypeptides, protein A or protein G affinity chromatography can be suitably used (see e.g. U.S. Pat. Nos. 4,801,687 and 5,151,504). Following capture using affinity chromatography, orthogonal polishing steps with appropriate process parameters may be used to remove any remaining process-related impurities such as HCP, and DNA. In general, to obtain a purified bispecific antibody or multivalent multimer, several steps are undertaken, comprising host cell culture, harvest clarification, followed by protein capture, anion exchange chromatography, to remove host cell DNA, and CIEX to remove host cell protein (HCP), leached protein A and potential aggregates followed by additional steps, such as virus filtration. Persons of skill in the art are aware the order of such steps may be modified or individual steps substituted. For example, alternatives for polishing steps include hydrophobic interaction chromatography and mixed-mode chromatography.

Said methods of processing bispecific proteins, or the like, in addition to the processing as described above, may further comprise a separation step of separating the produced bispecific proteins from the produced monospecific proteins (or multispecific from other produced proteins) based on the differences in isoelectric points between the produced bispecific protein and produced monospecific proteins. Any suitable separation step may be employed. A suitable separation step selected may be isoelectric focusing. Alternatively, or in addition, said method comprising a separation step of separating the produced bispecific proteins from the produced parent proteins, comprises ion-exchange or hydrophobic interaction. As shown in the example section, variations of non-surface exposed amino acids within the CH region, preferably buried amino acids, allows for a differentiation with regard to the charges, and can provide for differentiation in isoelectric points and/or chromatographic properties between bispecific proteins and parent proteins. Such differentiation allows for separation of these proteins using conventional chromatography, which includes ion-exchange and hydrophobic interaction. Preferred methods are industrial applicable separation methods for processing of pharmaceutical biological products, such as antibodies. Alternative methods of separation are included within the scope of the invention that utilize the charge and/or the isoelectric point (pI) differentials generated by use of the separation domains and variations, including for example, capillary zone and capillary isotachophoresis, and capillary isoelectric focusing, which are techniques know to persons of ordinary skill in the art.

As said, although the variations of the separation domains, such as a CH region, on its own as described herein may allow for sufficient separation of parent proteins from bispecific proteins in the methods as described herein, the formation of bispecific proteins during production in a cell may be promoted e.g. by varying CH3 regions as comprised in the CH-containing immunoglobulin polypeptides. Hence, a further method in accordance with the invention is provided wherein the first CH-containing immunoglobulin polypeptide and the second CH-containing immunoglobulin polypeptide comprise CH3 regions and wherein said CH3 regions comprise CH3 variations enhancing pairing between the first and second CH-containing immunoglobulin polypeptides. Preferably, one of the first and second CH-containing immunoglobulin polypeptides comprises CH3 variations L351D and L368E, and the other comprises CH3 variations T366K and L351K. DEKK residues are at the interface between the two domains that interact with each other to promote heterodimerization of a DE and KK chain, whereas two KK modified CH3 domains are repulsive. It is understood that, as described above, the DEKK variations are preferably selected to be aligned (i.e. adding a positive or negative charge to both CH3 and CH regions comprised in the same polypeptide). Other forms of heterodimerization technology are known in the art and can be employed with the variations described herein, for example using a knob-into-hole technology or an electrostatic engineering approach.

In the methods in accordance with the invention as described above for producing bispecific proteins, variations of the CH region are preferably variations as defined throughout herein as being suitable for the bispecific proteins, and preferably, said bispecific protein preferably can be selected to comprise the further features as described throughout herein as well.

Preferably, said proteins produced in the methods of the invention are bispecific antibodies, which are more preferably human bispecific antibodies, most preferably of human IgG1. Said bispecific proteins as produced in a method in accordance with the invention, wherein the CH region of the immunoglobulin polypeptide is selected to be a CH region from a human IgG1, and the amino acids within the CH region comprise a charge differential from a human wild type CH region at positions selected from the group consisting of T120, K147, D148, N159, Q175, N201, K213, V303, K370, E382, E388 as these amino acid positions as exemplified in the example section allow for alternative residues at these positions comprising a different charge (changing between a neutral, positively and negatively charged amino acid). Most preferred are the amino acids N159 and N201, which represent buried amino acids. Most preferred are the amino acids V303, E382, E388, which represent buried amino acids. Most preferably, said first and second CH-containing immunoglobulin polypeptides represent different heavy chains providing for different antigen binding domains, i.e. differing mainly with regard to the heavy chain variable regions.

In another embodiment, a bispecific or multispecific protein as produced in accordance with the invention comprises a first CH-containing immunoglobulin polypeptide and a second CH-containing immunoglobulin polypeptide, said CH region being a human IgG1 CH region, wherein the first or second CH-containing immunoglobulin polypeptides comprise one or more variations of amino acids selected from amino acids within the CH region, said variations comprising one or more variations selected from the group consisting of K147E, N159D, Q175E, N201D, K213Q, V303E, K370S, K370T or one or more variations selected from the group consisting of T120K, D148K, N159K, Q175K, N201K, V303K, E382Q, E382T, E388L, E388M, E388T. Preferably said isolated immunoglobulin protein is a bispecific antibody or multispecific antibody.

In a further embodiment, a bispecific or multispecific protein is produced in a method in accordance with the invention comprising a first CH-containing immunoglobulin polypeptide and a second CH-containing immunoglobulin polypeptide, said CH region being a human IgG1 CH region, wherein the first CH-containing immunoglobulin polypeptide comprises one or more variations of amino acids selected from amino acids within the CH region, said variations comprising one or more variations selected from the group consisting of K147E, N159D, Q175E, N201D, K213Q, V303E, K370S, K370T, and wherein the second CH-containing immunoglobulin polypeptide comprises one or more variations of amino acids selected from amino acids within the CH region, said variations comprising one or more variations selected from the group consisting of T120K, D148K, N159K, Q175K, N201K, V303K, E382Q, E382T, E388L, E388M, E388T. Most preferably said produced immunoglobulin protein is a bispecific or multispecific antibody.

In another embodiment, a bispecific or multispecific protein is produced in a method in accordance with the invention comprising a first CH1-containing immunoglobulin polypeptide and a second CH1-containing immunoglobulin polypeptide, said CH1 region being a human IgG1 CH1 region, wherein the first or second CH1-containing immunoglobulin polypeptide comprises one or more variations of amino acids selected from amino acids within the CH1 region, said variations selected from the group consisting of K147E and Q175E; N201D and K213Q; T197D and K213Q; N159D and K213Q; and K213Q, or said variations selected from the group consisting of T120K; N201K; D148K and Q175K; and N159K and a variation of an amino acid at a hinge residue E216K. Most preferably said produced bispecific or multispecific protein is a bispecific or multispecific human antibody.

In still a further embodiment, a bispecific or multispecific protein is produced in a method in accordance with the invention comprising a first CH1-containing immunoglobulin polypeptide and a second CH1-containing immunoglobulin polypeptide, said CH1 region being a human IgG1 CH1 region, wherein the first CH1-containing immunoglobulin polypeptide comprises one or more variations of amino acids selected from amino acids within the CH1 region, said variations selected from the group consisting of K147E and Q175E; N201D and K213Q; T197D and K213Q; N159D and K213Q; and K213Q and wherein the second CH1-containing immunoglobulin polypeptide comprises one or more variations of amino acids selected from amino acids within the CH1 region, said variations selected from the group consisting of T120K; N201K; D148K and Q175K; and N159K a variation of an amino acid at a hinge residue E216K. Most preferably said produced protein is a bispecific or multispecific human antibody.

A CH1, CH2 or CH3 region, further referred to as CH region comprising a variation of a neutral amino acid to a negatively charged amino acid; a positively charged amino acid to a neutral amino acid; and/or a positively charged amino acid to a negatively charged amino acid is said to be a CH region with a negative charge difference with respect to the original CH region, preferably as compared to a human wild-type CH region. The variation provides the negative charge difference to the CH region at the relevant pH. A CH region with a variation of a neutral amino acid to a positively charged amino acid; a negatively charged amino acid to a neutral amino acid; and/or a negatively charged amino acid to a positively charged amino acid is said to be a CH region with a positive charge difference with respect to the original CH region, preferably as compared to a human wild-type CH region. If a CH region has two variations of an amino acid residue as described herein it is preferred that both variations provide the same charge difference in kind to the CH region. If a CH region has three or more variations of an amino acid residue as described herein it is preferred that net result of the variations provide a charge difference to the CH region. The immunoglobulin region is preferably a human immunoglobulin region. In some embodiments the immunoglobulin region is an IgG region, preferably an IgG1 region. The immunoglobulin regions disclosed above can be used advantageously as a part of an antibody that needs to be separated from a mixture of antibodies.

The invention further provides an antibody comprising a heavy chain and a light chain comprising an immunoglobulin CH region as described herein. For instance when such an antibody is produced as part of a mixture, the variation in charge provided to a CH region may facilitate separation of said antibody from said mixture. In a preferred embodiment the antibody comprises different heavy chains. In a preferred embodiment the antibody is a multispecific antibody such as a bispecific or trispecific antibody. In this case the variation in charge provided to a CH region may facilitate separation of said bispecific or trispecific antibody from said mixture. The different heavy chains preferably comprise compatible heterodimerization regions, preferably compatible heterodimerization CH3 regions. In one embodiment one of heavy chains comprises the CH3 variations L351D and L368E, and the other of said heavy chains comprises the CH3 variations T366K and L351K. The antibody is preferably an IgG antibody, preferably an IgG1 antibody. In some embodiments the antibody comprises two or more immunoglobulin CH regions as described herein. It is preferred that the heavy chain that comprises the CH3 variations L351D and L368E comprises one CH region as described herein and that the heavy chain that comprises the CH3 variations T366K and L351K comprises another CH region as described herein. In such cases it is preferred that the one and the other CH regions comprise CH regions with different charges. In such cases the difference in iso-electric points of the resulting antibodies in the mixture will be further apart thereby facilitating separation of said antibody from said mixture. In other words if one CH region is a CH region with a negative charge difference with respect to the original CH region the other is preferably a CH region with a positive charge difference with respect to the original CH region. Similarly if one CH region is a CH region with a positive charge difference with respect to the original CH region the other is preferably a CH region with a negative charge difference with respect to the original CH region. The CH3 variations L351D and L368E, and the CH3 variations T366K and L351K preferably match the charge difference of the CH variation. The variations L351D and L368E are preferably in the heavy chain comprising the CH region with a negative charge difference with respect to the original CH region or comparative residue in the original or native CH region. The variations T366K and L351K are preferably in the heavy chain comprising the CH region with a positive charge difference with respect to the original CH region or comparative residue in the original or native CH region. For example a polypeptide comprising CH3 variations L351D and L368E may be combined with one or more of the following K147E, N159D, Q175E, N201D, K213Q, V303E, K370S, K370T, or other variations that increase the negative charge of the polypeptide as set out herein. Similarly, a polypeptide comprising CH3 variations T366K and L351K may be combined with one or more of the following variations T120K, D148K, N159K, Q175K, N201K, V303K, E382Q, E382T, E388L, E388M, E388T, or other variations that increase the positive charge of the polypeptide as set out herein.

Antibodies with compatible heterodimerization regions such as compatible CH13 heterodimerization regions as described herein with such CH1 regions typically separate better from the respective antibodies having the same heavy chains, and/or half antibodies, if present, in a separation step that utilize charge and/or the isoelectric point (pI) of antibodies or fragments thereof. The antibody preferably comprises one or more light chains. It preferably comprises the same light chain. The light chain is preferably a common antibody light chain as described herein. The common light chain preferably comprises a light chain variable region as depicted in FIG. 13B or FIG. 13D. In one embodiment the light chain has a light chain constant region as depicted in FIG. 13C. In a preferred embodiment the light chain has an amino acid sequence of a light chain depicted in FIG. 13A or FIG. 13E. The common light is preferably a light chain having the CDRs as depicted in FIG. 13F. The antibody or CH region is preferably a human antibody or human immunoglobulin CH region, wherein the human CH region comprises variations at amino acid(s) positions that are non-surface exposed, and preferably buried within wild-type human CH regions.

The immunoglobulin region, preferably a CH1 region or antibody comprising a variation of an amino acid that is not surface exposed and preferably buried as described herein, preferably has a variation that is selected from amino acids that are not present at the CH1/CL interface. The Q175 position is in the CH1/CL interface but is nevertheless exceptionally effective and stable.

The immunoglobulin region, preferably a CH3 region or antibody comprising a variation of an amino acid that is not surface exposed and preferably buried as described herein, preferably has a variation that is selected from amino acids that are not present at the CH3 interface. The K370 position is the exception. It is in the CH3/CH3 (see FIG. 22). Nevertheless it is a good position for introducing a variation as indicated herein, even without compensating variations are the opposing CH3 chain, such as for instance present in the DEKK.

The immunoglobulin region, preferably a CH1, CH2 or CH3 region or antibody comprising a variation of an amino acid that is not surface exposed as described herein, preferably does not substantially, adversely affect the stability of the resulting CH1 region or antibody, including any heavy and light chain interface. The immunoglobulin region, preferably a CH1, CH2 or CH3 region or antibody comprising a variation of an amino acid that is not surface exposed as described herein, may include additional variation(s) that bolster stability of the variation(s) that produce a charge difference.

The immunoglobulin region, preferably a C1 region or antibody comprising a variation of an amino acid that is not surface exposed as described herein, may include additional variation(s) that produce a charge difference.

The invention further provides a method of producing an antibody of any one of the above, wherein the method comprises the steps of

providing a nucleic acid encoding a first heavy chain with a CH region as described herein;
providing a nucleic acid encoding a second heavy chain, wherein said first and second heavy chain may be the same or different;
providing a nucleic acid encoding a light chain;
introducing said nucleic acid into host cells and culturing said host cells to express the nucleic acid(s); and
collecting the antibody from the host cell culture, the method further comprising separation of the antibody from other antibodies or antibody fragments in a separation step based on the electrical charge of the antibodies and/or antibody fragments. In one embodiment said first and second heavy chains comprise compatible heterodimerization regions, preferably compatible CH3 heterodimerization regions.

The invention further provides a method of producing an antibody of any one of the above, wherein the method comprises the steps of

providing a nucleic acid encoding a first heavy chain with a CH region as described herein;
providing a nucleic acid encoding a second heavy chain, wherein said first and second heavy chain may be the same or different;
providing a nucleic acid encoding a light chain;
introducing said nucleic acid into host cells and culturing said host cells to express the nucleic acid(s); and
collecting the antibody from the host cell culture, the method further comprising performing a harvest clarification,
performing protein capture,
performing anion exchange chromatography, and
performing cation exchange chromatography to separate the antibody from other antibodies or antibody fragments. In one embodiment said first and second heavy chains comprise compatible heterodimerization regions, preferably compatible CH3 heterodimerization regions.

The invention further provides a method of producing an antibody of any one of the above, wherein the method comprises the steps of

providing a nucleic acid encoding a first heavy chain with a CH region as described herein;
providing a nucleic acid encoding a second heavy chain, wherein said first and second heavy chain may be the same or different;
providing a nucleic acid encoding a light chain;
introducing said nucleic acid into host cells and culturing said host cells to express the nucleic acid(s); and
collecting the antibody from the host cell culture, the method further comprising separating the antibody from other antibodies or antibody fragments in a separation step comprising isoelectric focusing on a gel.

Further provided is a method for producing a multispecific antibody comprising a first heavy chain and a second heavy chain whose isoelectric points are different, wherein the method comprises the steps of:

(a) expressing a nucleic acid encoding a first heavy chain and a nucleic acid encoding a second heavy chain, such that isoelectric points of the encoded first heavy chain and that of the encoded second heavy chain differ, wherein said nucleic acid encodes one or more variations at amino acid position(s) selected from non-surface exposed positions of an encoded immunoglobulin region comprising a first and/or second heavy chain, preferably a CH1 region, a CH2, a CH3, more preferably, T120, K147, D148, Y149, V154, N159, A172, Q175, S190, N201, K213, V303, K370, E382 and E388 (EU-numbering in the CH region) and
(b) culturing host cells to express the nucleic acid; and
(c) collecting the multispecific antibody from the host cell culture, using the difference in isoelectric point.

Also provided is a method for separating a multispecific antibody comprising a first heavy chain and a second heavy chain whose isoelectric points are different, wherein the method comprises the steps of:

(a) expressing both or either one of a nucleic acid encoding the amino acid residues of the first heavy chain and a nucleic acid encoding the amino acid residues of the second heavy chain, such that the isoelectric point of the encoded first heavy chain and that of the encoded second heavy chain differ, wherein the position(s) of said nucleic acid is/are position(s) that differ from an encoded CH region at a non-surface exposed residue(s), preferably one or more amino acid variations selected from T120, K147, D148, Y149, V154, N159, A172, Q175, S190, N201, K213, V303, K370, E382 and E388 (EU-numbering in the CH region) and
(b) culturing host cells to express the nucleic acid; and
(c) separating the multispecific antibody from the host cell culture by chromatography.

In a preferred embodiment the nucleic acid encodes a first heavy chain and second heavy chain, such that a retention time of the first heavy chain, a homomultimer of the first heavy chain, the second heavy chain, a homomultimer of the second heavy chain, and a heteromultimer of the first and second heavy chain differ when expressed and are separated in an ion exchange chromatography step.

The variant amino acid(s) at the position(s) encoded by said nucleic acid is/are preferably selected from amino acids that are non-surface exposed in a human wild-type CH region and selected from
a neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid.

Also provided is a method for producing a multispecific antibody comprising a first heavy chain and a second heavy chain whose isoelectric points are different, wherein the method comprises the steps of:

providing a nucleic acid encoding a CH region of the first heavy chain and a nucleic acid encoding a CH region of the second heavy chain, such that the isoelectric point of the first encoded heavy chain and that of the second encoded heavy chain differ, wherein at least one of said CH regions comprises an amino acid variation in a CH region at a position selected from T120, K147, D148, Y149, V154, N159, A172, Q175, S190, N201, K213, V303, K370, E382 and E388 (EU-numbering) and

culturing host cells to express the nucleic acid; and

collecting the multispecific antibody from the host cell culture, using the difference in isoelectric point further comprising the steps of

collecting the antibody from the host cell culture,

performing harvest clarification,

performing protein capture,

performing anion exchange chromatography, and

performing cation exchange chromatography to separate the antibody from another antibody or an antibody fragment.

Further provided is a method for purifying a multispecific antibody comprising a first heavy chain and a second heavy chain whose isoelectric points are different, wherein the method comprises the steps of:

providing both or either one of a nucleic acid encoding a CH region of the first heavy chain and a nucleic acid encoding a CH region of the second heavy chain, such that the first encoded heavy chain and the second encoded heavy chain differ in isoelectric point, wherein at least one of said CH regions comprises an amino acid variation at a position selected from T120, K147, D148, Y149, V154, N159, A172, Q175. S190, N201, K213, V303, K370, E382 and E388 3 (EU-numbering of the CH region) and

culturing host cells to express the nucleic acid; and

purifying the multispecific antibody from the host cell culture by isoelectric focusing and separating the multispecific antibody from another antibodies or an antibody fragment.

The one or more nucleic acid encoding a homomultimer of the first heavy chain, a homomultimer of the second heavy chain, and a heteromultimer of the first and second heavy chain are expressed as proteins having different isoelectric points and produce different retention times in ion exchange chromatography.

The invention further provides a method for producing or purifying an antibody such as a multispecific antibody as described wherein which further comprises determining the charge or pI difference of the heavy chains relative to each other and selecting the heavy chain with the more negative charge/pI as said first heavy chain and the heavy chain with more the positive charge/pI as said second heavy chain. This embodiment additionally facilitates the separation of the multispecific antibody from homodimers and halfbodies in a charge separation method such as CIEX. As indicated herein above said first heavy chain preferably comprises one or more CH1, CH2 or CH3 regions as described herein that provide an additional negative charge to the heavy chain. Similarly as indicated herein above said second heavy chain preferably comprises one or more CH1, CH2 or CH3 regions as described herein that provide an additional positive charge to the heavy chain. In addition and as also referred to herein above, the first heavy chain preferably comprises a DE variations of the CH3 heterodimerization domain whereas said second heavy chain preferably comprises the KK variations of the CH3 heterodimerization domain.

In these embodiments the natural charge difference between the heavy chains, the amino acid variations of the CH1, CH2 and/or CH3 regions described herein and optionally the charge difference introduced by DEKK CH3 heterodimerization domain as described herein all work together to improve charge separation of the antibody such as the bispecific and multispecific antibody described herein.

A factor that may result in a difference in the relative charge of the two heavy chains is a difference in the amino acid sequence of the variable domains. For instance a difference in the amino acid sequence of the heavy chain variable regions when the same light chain is used for both heavy chain variable regions. In such cases it is often sufficient to determine the charge or pI difference of the variable domains or of the heavy chain variable regions as the case may be relative to each other.

The charge or pI difference of variable domains can be used to improve the production and/or purification as indicated above. In some embodiments variable domains heavy different heavy chains and the same light chain. Examples of light chains that can be used as such are described elsewhere herein and some are for instance listed in FIG. 13A-13P. Heavy chain variable regions that can be used in such a method are typically selected to pair well with the selected light chain. Heavy chain variable region that are selected to pair well with the light chain of FIG. 13A are described in the examples. Other examples of such heavy chains variable regions are described in WO2015/130172; PCT/NL2020/050081; WO2019/031965; WO2019/009726; WO2019/009728; and WO2019/009727 which are enclosed by reference herein for this purpose. The heavy chain variable regions as mentioned herein and described in the above references are to be considered as suitable examples of heavy chains and not considered to be a limitative list. The invention can be applied to a large variety of variable domains and/or heavy light chain combinations. Some examples of such variable domains and/or heavy light chain combinations are depicted in FIGS. 1A-1E and 2A-2B and description thereof. Other examples of heavy and light chain combinations are for instance described in WO2019190327 which is referred to by reference herein for that purpose.

### BRIEF DESCRIPTION OF THE DRAWINGS

FIG. 1A-1E. Schematic representations of bispecific and monospecific antibodies are provided in accordance with separation domains of the invention. It should be noted that other features and aspects of the invention are apparent from the detailed description, taken in conjunction with the accompanying drawings which illustrate, by way of example, the features in accordance with embodiments of the invention. Each of the figures provided is exemplary and is not intended to nor do these figures limit the scope of the inventions provided, which are defined by the claims and the full extent of the detailed disclosure, which describe and enable the inventions set out herein. In FIGS. 1A-1C, the first CH1-containing immunoglobulin is depicted in black, representing a first heavy chain, and a second CH1-containing immunoglobulin is depicted in grey, representing a second heavy chain, and in white, the light chain is depicted, in the scenario being a common light chain. In these figures, a first heavy chain comprises a separation CH1 region (FIG. 1A), a second heavy chain comprises a separation CH1 region (FIG. 1B), and both a first and second heavy chain comprise a separation CH1 region of alternative charges (FIG. 1C). Again, it is understood that the invention does not require the use of a common light chain, which is depicted as an example of an embodiment of the invention. In FIG. 1D, the first CH2-containing immunoglobulin is depicted in black, representing a first heavy chain, and a second CH2-containing immunoglobulin is depicted in grey, representing a second heavy chain, and in white, the light chain is depicted, wherein the second heavy chain comprises a separation CH2 domain. In FIG. 1E, a single heavy chain is used and depicted in black, with two different light chains depicted in grey and white. The variations are indicated with either + or −, indicating the relative change of charge as compared with an unmodified or reference domain, and the integration of the respective + and − signs for the unmodified or reference antibody. In FIGS. 1A-1C, the CH1 region comprises separation residues of the invention described herein, in 1D the CH2 region, and in FIG. 1E, the CL region of the light chain is a variant light chain in accordance with the invention set forth, FIG. 1A) In this scenario, the first heavy chain is provided with a positive charge (indicated with + in the CH1 region), this results in the two monospecific antibodies having either a ++ or neutral charge, wherein the bispecific antibody has a + charge. The charge as indicated represents the change in charge as compared with antibodies lacking the separation domains. FIG. 1B) In this scenario, the second heavy chain is provided with two negative charges (indicated with −− in the CH1 region), this results in the two monospecific antibodies having either a −−−− or neutral charge, wherein the bispecific antibody has a −− charge. FIG. 1C) In this scenario, the first heavy chain is provided with a positive charge (indicated with + in the CH1 region), the second heavy chain is provided with a negative charge (indicated with − in the CH1 region), this results in the two monospecific antibodies having either a −− or ++ charge, wherein the bispecific antibody has a neutral charge. FIG. 1D) In this scenario, the first heavy chain lacks a separation domain, and the second heavy chain comprises a negative separation CH2 domain having a −2 charge variation. This results in two monospecific antibodies having a neutral or −−−− charge, whereas the bispecific antibody has a −− charge. FIG. 1E) In this scenario, two CL domains are employed, one comprising a positive CL separation domain and one that is not a separation domain, lacking variation. The format depicted here utilizes a common heavy chain format. This results in monospecific antibodies having ++ or neutral charge, whereas the bispecific antibody has a + charge.

FIG. 2A-2B. Schematic representation of mono-, trispecific or trivalent, and quadrispecific or quadrivalent antibodies are provided in accordance with the invention. In FIGS. 2A and 2B, the first CH-containing immunoglobulin is depicted in black, representing a first heavy chain, having in addition a second CH1-VH domain (black and striped) via a linker. The second heavy chain is depicted in grey. The common light chain is depicted in white. Again, it is understood that the invention does not require the use of a common light chain, which is depicted as an example of an embodiment of the invention. The variations are indicated with either + or −, indicating the relative change of charge as compared with an unmodified chain, or unmodified antibody. In FIG. 2A) the CL region of the light chain is a separation domain, and in 213) the CH1 region of the first heavy chain is a separation domain. In FIG. 2A), in this scenario, a quadrispecific or quadrivalent antibody having a −−−− charge and a monospecific antibody having a −− charge is formed, whereas the trispecific antibody has a −−− charge is formed. In FIG. 2B), a quadrispecific or quadrivalent antibody is formed having a −−−−−−−− charge and a monospecific antibody has a neutral charge, whereas the trispecific or trivalent antibody has a −−−− charge is formed.

FIG. 3. Melting curves of monospecific antibodies are provided setting out two peaks associated with such antibody having a wildtype CH1 and those comprising a variant CH1 region.

FIG. 4. Isoelectric focusing of bivalent monospecific antibodies produced having CH1 variations is provided, demonstrating separation in bands based on charge. These data show a correlation between separation domain increasing or decreasing charge and the corresponding capacity to separate in bands antibodies comprising these domains during isoelectric focusing.

FIG. 5. CIEX chromatography of DE, KK and DE and KK arms. The upper graph shows a chromatogram of a monospecific, bivalent antibody produced using a DE arm having a wild-type CH1 sequence and a heavy chain variable region (MF1516). The lower graphs show a chromatogram of a monospecific, bivalent antibody produced using a KK arm having a wild-type CH1 sequence and a different heavy chain variable region (MF3462). The middle graph shows a chromatogram of a bispecific antibody produced using above mentioned KK arm having a wild-type CH1 sequence and above mentioned DE arm having a wild-type CH1 sequence. In the upper graph, the arrow indicates the bivalent monospecific antibody produced (DE/DE) and in the lower graph the arrow indicates the monovalent monospecific “halfbody” produced (KK). The light chain for each antibody is the same.

FIG. 6. CIEX chromatography of DE, KK and DE and KK arms with a separation CH1 region. The upper graph shows a chromatogram of a monospecific, bivalent antibody produced using a DE arm having a CH sequence with T197D and K213Q variations and a heavy chain variable region (MF1516). The lower graphs shows a chromatogram of a monospecific, bivalent antibody produced using a KK arm having a CH1 sequence with N159K and a hinge residue E216K variations and a heavy chain variable region (MF3462). The middle graph shows a chromatogram of bispecific antibody (MF1516/MF3462) produced using the KK arm and the DE arm combined, and the separation of the peak for the bivalent DE. T197D, K213Q/KK, N159K, E216K from the other proteins formed. The light chain for each antibody is the same.

FIG. 7. Separation of bispecific antibodies—CIEX retention time CIEX chromatography of DE arms with wild type CH1 and KK arms with separation CH1 regions. The upper graph shows a chromatogram of antibody produced using DE and KK arms having a wild type CH1 sequence. The second graph shows a chromatogram of antibody produced using KK arms having a CH1 sequence with T120K and wild type CH1 region with the DE arms. The third graph shows a chromatogram of antibody produced using KK arms having a CH1 sequence with N201K and wild type CH1 region with the DE arms. The bottom graph shows a chromatogram of antibody produced using KK arms having a CH1 sequence with N159K and a hinge residue E216K and wild type CH1 region with the DE arms. The white arrow indicates the bivalent monospecific antibody produced (DE/DE). The black arrow indicates the bivalent bispecific antibody produced (DE/KK). The grey arrow indicates the bivalent monospecific antibody produced (KK/KK). The light chain for each antibody is the same.

FIG. 8. Separation of bispecific antibodies—CIEX retention time CIEX chromatography of DE arms with separation CH1 regions and KK arms with wild type CH1. The upper graph shows a chromatogram of antibody produced using DE and KK arms having a wild type CH1 sequence. The middle graph shows a chromatogram of antibody produced using DE arms having a CH1 sequence with T197D and K213Q and wild type CH1 region with the KK arms. The bottom graph shows a chromatogram of antibody produced using DE arms having a CH1 sequence with K213Q and wild type CH1 region with the KK arms. The white arrow indicates the bivalent monospecific antibody produced (DE/DE). The black arrow indicates the bivalent bispecific antibody produced (DE/KK). The grey arrow indicates the bivalent monospecific antibody produced (KK/KK). The light chain for each antibody is the same.

FIG. 9. Separation of bispecific antibodies—CIEX retention time CIEX chromatography of DE arms and KK arms with wild type or separation CH1 regions. The upper graph shows a chromatogram of antibody produced using DE and KK arms having a wild type CH1 sequence. The second graph shows a chromatogram of an antibody produced using KK arms having a CH1 sequence with T120K and DE arms having a CH sequence with T197D and K213Q. The third graph shows a chromatogram of an antibody produced using KK arms having a CH1 sequence with N201K and DE arms having a CH1 sequence with T197D and K213Q. The bottom graph shows a chromatogram of an antibody produced using KK arms having a CH1 sequence with N159K and a hinge residue E216K and DE arms having a CH1 sequence with T197D and K213Q. The white arrow indicates the bivalent monospecific antibody produced (DE/DE). The black arrow indicates the bivalent bispecific antibody produced (DE/KK). The grey arrow indicates the bivalent monospecific antibody produced (KK/KK). The light chain for each antibody is the same.

FIG. 10. Separation of bispecific antibodies—CIEX retention time

CIEX chromatography of DE arms and KK arms with wild type or a separation CH1 region. The upper graph shows a chromatogram of antibody produced using DE and KK arms having a wild type CH1 sequence. The second graph shows a chromatogram of antibody produced using KK arms having a CH1 sequence with T120K and DE arms having a CH1 sequence with K213Q. The third graph shows a chromatogram of antibody produced using KK arms having a CH1 sequence with N201K and DE arms having a CH1 sequence with K213Q. The bottom graph shows a chromatogram of antibody produced using KK arms having a CH1 sequence with N159K and a hinge residue E216K and DE arms having a CH1 sequence with K213Q. The white arrow indicates the bivalent monospecific antibody produced (DE/DE). The black arrow indicates the bivalent bispecific antibody produced (DE/KK). The grey arrow indicates the bivalent monospecific antibody produced (KK/KK). The light chain for each antibody is the same.

FIGS. 11A-11E.—CIEX retention time of monospecific, bivalent antibodies (MF1122/MF1122).

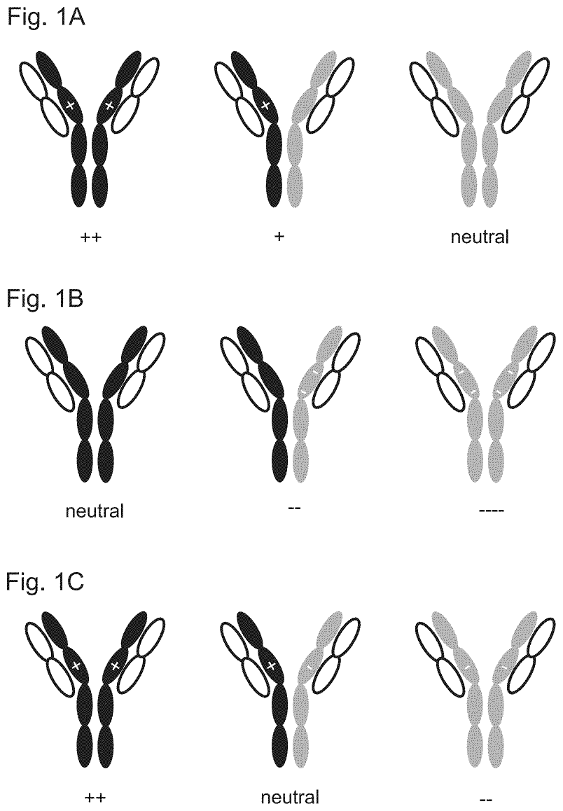

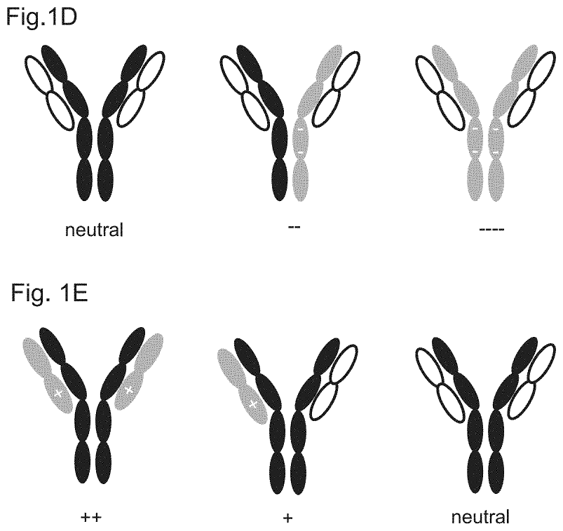

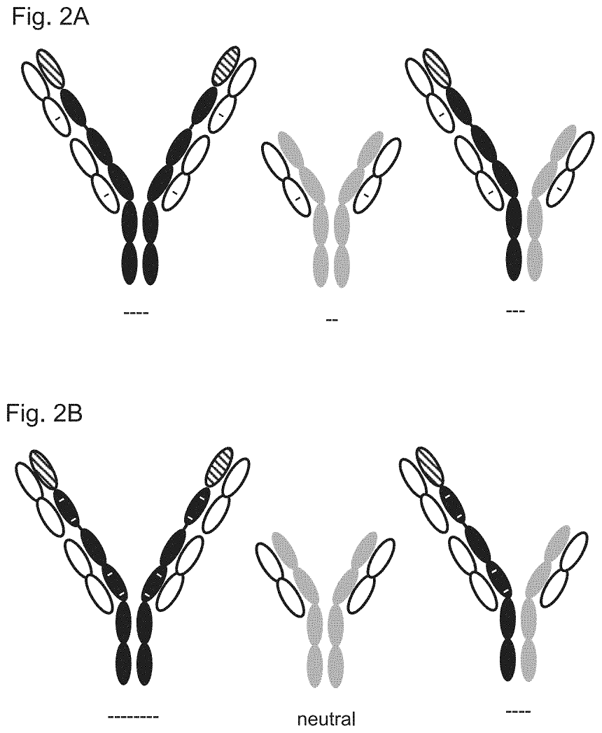

CIEX chromatography of monospecific antibodies having variations in the CH1 region Each variant is tested separately and graphs show the CIEX retention time for each variant demonstrating different retention times as compared to a monospecific, bivalent antibody comprising two human wild type CH1 regions. FIG. 1A shows the chromatographic results for variants T197D and K213Q; K213Q and N159D; and K147E and Q175E. FIG. 11B shows the chromatographic results for variants N201D and K213Q; K213Q; and N201D. FIG. 11C shows the chromatographic results for variants N201D, A172P, and S190A; wild-type; and variants Y149A, V154I, A172P, and S190A. FIG. 11D shows the chromatographic results for variants T120K; N201K; and N201K, A172P, and S190A. FIG. 11E shows the chromatographic results for variants D148K and Q175K; and N159K and E216K.

FIG. 12. structure of constructs used for cloning

Constructs used for cloning to prepare constructs for the expression of antibodies having separation CH1 regions. The CH2 and CH3 domain are obtained from the MV1708 construct. This construct contains a unique BspEI site at the N-terminus of CH2. The heavy chain variable domain (V) was obtained from the MF1122 construct. The CH1 region was cloned into the final construct flanked by a BstE and a BstE restriction site.

FIGS. 13A-13P

FIG. 13A Amino acid sequence common light chain;

FIG. 13B DNA and amino acid sequence of a common light chain variable domain (IGKV1-39/jk1);

FIG. 13C DNA and amino acid sequence of a common light chain constant region;

FIG. 13D Amino acid sequence IGKV1-39/jk5 common light chain variable domain;

FIG. 13E Amino acid sequence V-region IGKV1-39A;

FIG. 13F CDR1. CDR2 and CDR3 of a common light chain;

FIG. 13G Amino acid sequence of human common light chain IGKV3-15/jk1;

FIG. 13H Amino acid sequence of human common light chain IGKV3-20/jk1;

FIG. 13I Amino acid sequence of human common light chain IGLV3-21/jl3;

FIG. 13J Amino acid sequence of the V-region of IGKV3-15;

FIG. 13K Amino acid sequence of the V-region of IGKV3-20;

FIG. 13L Amino acid sequence of human common light chain IGKV1-39/jk5 and kappa constant region;

FIG. 13M Amino acid sequence of human common light chain IGKV3-15/jk1 and kappa constant region;

FIG. 13N Amino acid sequence of human common light chain IGKV3-20/jk1 and kappa constant region;

FIG. 13O Amino acid sequence of human common light chain IgVλ3-21/IGJλ3 and lambda constant region;

FIG. 13P Amino acid sequence of the V-region of IGLV3-21.

FIGS. 14A-14E. IgG heavy chains for the generation of bispecific molecules. FIG. 14A CH1 region. FIG. 14B hinge region. FIG. 14C CH2 region. FIG. 14D CH3 domain containing variations L351K and T366K (KK). FIG. 14E CH3 domain containing variations L351D and L368E (DE).

FIG. 15. A three-dimensional model of a human wild type CH1 region depicting 84 position 201 under EU numbering in dark grey and sharp lines at the arrow, demonstrating its buried position and lack of solvent accessibility within the core of the protein.

FIG. 16. ELISA results. Binding of fibrinogen or PD-L1 specific IgG1 antibodies with the indicated CH1 variants to fibrinogen and PD-L1. PG1122 is a monospecific bivalent fibrinogen binding antibody with two identical heavy and light chains. The two variable domains have a heavy chain variable region with the amino acid sequence of MF1122 and the light chain of FIG. 13A. The numbers p113, p118 etc indicate which amino acid variants the CH1 region of the antibody has. This information is provided in table 16. PG PD-L1 is a monospecific bivalent antibody with two identical PD-L1 binding variable domains. The numbers p06-p13 indicate which amino acid variants the CH1 region of the antibody has. This information is provided in table 16.

FIG. 17. IMGT table with EU-numbering of the respective amino acids of an IgG1 CH1, hinge, CH2 and CH3 region. Included for numbering purposes of amino acid residue positions.

FIG. 18. Summary of ELISA results with the bispecific antibodies and the indicated monospecific antibodies of FIG. 19. All tested bispecific antibodies bind c-MET and Tetanus Toxoid in a dose dependent manner.

FIG. 19. A summary of the characteristics of the antibodies tested. Each row lists one antibody. PB indicates an antibody with two different variable domains, PG indicates an antibody with two identical variable domains. A number following PB identifies the two variable domain combination, of which the heavy chain variable region is identified with the indication MG followed by a number. MG1516 . . . and MG3462 . . . in the next column indicates that one variable domain has a VH of MF1516 and the other a VH of MF3462. The light chain region was the light chain of FIG. 13A. NA is not applicable. Columns MG1 that do not mention NA indicate that this antibody has a heavy chain with a DE CH3 domain. Columns MG2 that do not mention NA indicate that this antibody has a heavy chain with a KK CH3 domain. WT IgG1 indicates that these antibodies have all wild type IgG1 constant regions, a light chain of FIG. 13A and heavy chain variable regions of MF1516 or MF3462. DEDE indicates that these antibodies have only a heavy chain with a DE CH3 domain. KK indicates that these antibodies have only a heavy chain with a KK CH3 domain.

FIG. 20. CIEX profiles of bispecific and monospecific antibodies. The codes of the respective antibodies are indicated above or below the respective panel. The left arrow indicated the DEDE homodimer. The arrow on the right the KK-halfbody. The antibody code is decoded in FIG. 19 and table 24.

FIG. 21. CIEX profiles of bispecific and monospecific antibodies. The codes of the respective antibodies are indicated above or below the respective panel. The left arrow indicated the DEDE homodimer. The arrow on the right the KK-halfbody. The antibody code is decoded in FIG. 19 and table 24.

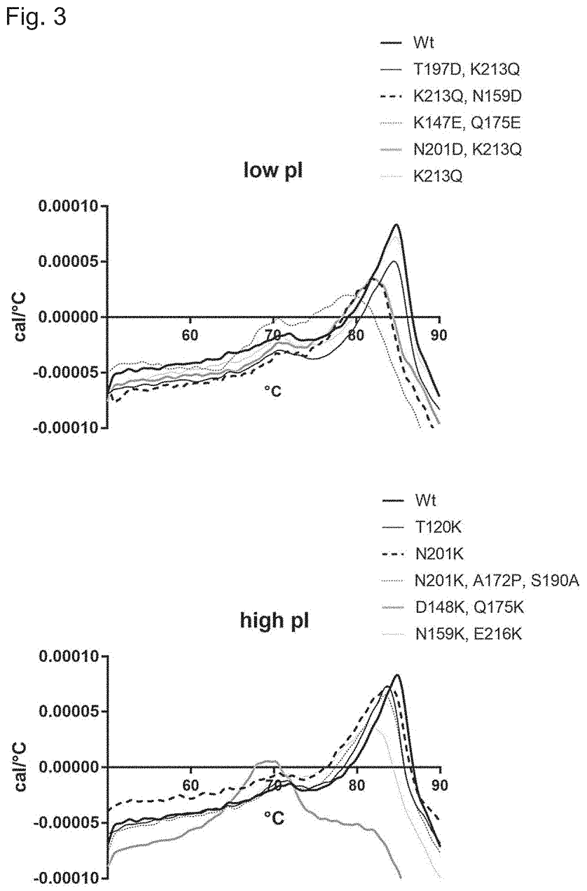

FIG. 22, CH3 residues that are identified to be at the interface of the CH3/CH3 homodimer according to Traxlmayer et al (2012). J Mol Biol. October 26; 423(3): 397-412. (see discussion and FIG. 3).

### PDB FILES

The application references four protein database files (PDB files). One for a CH1 region, one for a CH2 region and two for two different CH3 regions. One of the CH3 regions is the DE variant of the (11:3 heterodimerization domain, the other is the KK variant of the CH3 heterodimerization domain.

### EXAMPLES

### Example 1: Identifying Non-Surface Residues for Separation Design

From structural information of an IgG1 CH1 sequence with a VL domain, surface, non-surface exposed and buried amino acid residue positions within the CH1 region were identified by use of the program GETAREA 1.0 using default parameters. Negi et al., “Solvent Accessible Surface Areas. Atomic Solvation Energies, and Their Gradients for Macromolecules”, Last modified on Wed 17th April, 3:00 PM, 2015. A model of the CH1-CL domain with the sequence of table 1 and FIG. 13C was submitted to the Swiss-model website (Arnold K. Bordoli L, Kopp J, Schwede T. The SWISS-MODEL workspace: a web-based environment for protein structure homology modelling. Bioinformatics, 2006 Jan. 15:22(2):195-201). A high quality homology model was obtained by aligning (with greater than 95% identity over the full length of the CH1 region) to PDB structure GC6X.pdb (A 1.99 Å crystal structure of Middle-East Respiratory Syndrome coronavirus neutralizing antibody JC57-14 isolated from a vaccinated rhesus macaque). Numerous other CH1 regions in the PDB could provide high quality starting structures (with >95% sequence identity to the CH1 region used here and high quality structures). This structure was processed with GETAREA 1.0 beta, uploading the pdb file generated to determine the percent of each residue's surface area predicted to be accessible to solvent. Based on the default parameters of GETAREA 1.0 beta, amino acids that are greater than 50% surface exposed are referred to as “Out” or surface, non-OUT or non-surface exposed residues are between 50% to greater than 20%, and less than 20% accessible are referred by GETAREA 1.0 beta as “In” or as referred to herein as buried amino acids (see table 1).

TABLE 1a
Probe radius: 1.600

| Residue | | Total | Apolar | Backbone | Sidechain | Ratio (%) | In/Out |
|---|---|---|---|---|---|---|---|
| ALA | 1 | 96.46 | 36.86 | 86.27 | 10.19 | 15.7 | i |
| SER | 2 | 100.14 | 63.98 | 14.57 | 85.57 | 100.8 | o |
| THR | 3 | 71.48 | 25.80 | 20.58 | 50.90 | 47.9 | |
| LYS | 4 | 88.08 | 83.61 | 4.27 | 83.82 | 51.0 | o |
| GLY | 5 | 15.60 | 8.43 | 15.60 | 0.00 | 17.9 | i |
| PRO | 6 | 8.38 | 0.00 | 8.38 | 0.00 | 0.00 | i |
| SER | 7 | 44.54 | 23.43 | 10.17 | 34.38 | 44.4 | |
| VAL | 8 | 3.70 | 0.00 | 3.70 | 0.00 | 0.0 | i |
| PHE | 9 | 101.92 | 101.92 | 0.00 | 101.92 | 56.6 | o |
| PRO | 10 | 63.90 | 49.02 | 14.88 | 49.02 | 46.6 | |
| LEU | 11 | 72.30 | 72.23 | 6.15 | 66.15 | 45.2 | |
| ALA | 12 | 61.03 | 43.83 | 17.20 | 43.83 | 67.5 | o |
| PRO | 13 | 14.19 | 2.80 | 11.39 | 2.00 | 2.7 | i |
| SER | 14 | 66.46 | 53.17 | 14.10 | 52.36 | 67.6 | o |
| SER | 15 | 129.87 | 74.67 | 44.68 | 85.18 | 100.0 | o |
| LYS | 16 | 199.56 | 132.92 | 29.10 | 170.46 | 100.0 | o |
| SER | 17 | 26.37 | 12.21 | 21.94 | 4.43 | 5.7 | i |
| THR | 18 | 98.88 | 65.82 | 8.23 | 90.65 | 85.4 | o |
| SER | 19 | 118.70 | 62.69 | 24.19 | 94.51 | 100.0 | o |
| GLY | 20 | 75.24 | 38.21 | 75.24 | 0.00 | 86.3 | o |
| GLY | 21 | 23.73 | 22.10 | 23.73 | 0.00 | 27.2 | |
| THR | 22 | 90.44 | 44.60 | 16.22 | 74.22 | 69.9 | o |
| ALA | 23 | 2.96 | 2.96 | 2.96 | 0.00 | 0.0 | i |
| ALA | 24 | 55.34 | 49.03 | 6.31 | 49.03 | 75.5 | o |
| LEU | 25 | 2.47 | 2.47 | 2.47 | 0.00 | 0.0 | i |
| GLY | 26 | 0.00 | 0.00 | 0.00 | 0.00 | 0.0 | i |
| CYS | 27 | 0.00 | 0.00 | 0.00 | 0.00 | 0.0 | i |
| LEU | 28 | 50.30 | 50.30 | 0.00 | 50.30 | 34.4 | |
| VAL | 29 | 0.00 | 0.00 | 0.00 | 0.00 | 0.0 | i |
| LYS | 30 | 72.65 | 54.33 | 0.00 | 72.65 | 44.2 | |
| ASP | 31 | 27.99 | 2.43 | 0.00 | 27.99 | 24.8 | |
| TYR | 32 | 0.00 | 0.00 | 0.00 | 0.00 | 0.0 | i |
| PHE | 33 | 54.61 | 54.61 | 0.00 | 54.61 | 30.3 | |
| PRO | 34 | 44.56 | 44.56 | 0.00 | 44.56 | 42.4 | |
| GLU | 35 | 75.13 | 35.01 | 10.96 | 64.17 | 45.4 | |
| PRO | 36 | 91.37 | 90.40 | 0.98 | 90.40 | 85.9 | o |
| VAL | 37 | 12.46 | 0.09 | 12.38 | 0.08 | 0.1 | i |
| THR | 38 | 81.50 | 74.42 | 3.01 | 77.69 | 73.2 | o |
| VAL | 39 | 19.62 | 0.07 | 19.62 | 0.00 | 0.0 | i |
| SER | 40 | 43.68 | 37.75 | 4.85 | 38.83 | 50.2 | o |
| TRP | 41 | 0.05 | 0.06 | 0.00 | 0.05 | 0.0 | i |
| ASN | 42 | 26.68 | 16.57 | 11.74 | 14.94 | 13.1 | i |
| SER | 43 | 101.97 | 52.17 | 29.16 | 72.81 | 94.1 | o |
| GLY | 44 | 40.36 | 25.94 | 40.36 | 0.00 | 46.3 | |
| ALA | 45 | 92.86 | 67.14 | 40.49 | 52.36 | 80.7 | o |
| LEU | 46 | 36.55 | 34.54 | 2.63 | 33.92 | 23.2 | |
| THR | 47 | 101.76 | 68.88 | 15.06 | 86.70 | 81.6 | o |
| SER | 48 | 99.15 | 64.33 | 14.13 | 85.02 | 100.8 | o |
| GLY | 49 | 34.74 | 27.90 | 34.74 | 0.00 | 39.8 | |
| VAL | 50 | 36.27 | 17.50 | 18.77 | 17.50 | 14.3 | i |
| HIS | 51 | 117.55 | 88.13 | 4.51 | 113.04 | 73.1 | o |
| THR | 52 | 57.30 | 32.86 | 22.85 | 34.46 | 32.4 | |
| PHE | 53 | 106.87 | 106.72 | 0.99 | 105.88 | 58.8 | o |
| PRO | 54 | 123.36 | 121.33 | 14.74 | 108.62 | 100.0 | o |
| ALA | 55 | 19.73 | 0.64 | 19.10 | 0.63 | 1.0 | i |
| VAL | 56 | 110.89 | 110.89 | 3.68 | 107.21 | 87.7 | o |
| LEU | 57 | 102.18 | 83.44 | 18.74 | 83.44 | 57.1 | o |
| GLN | 58 | 44.42 | 33.86 | 10.63 | 33.79 | 23.5 | |
| SER | 59 | 142.95 | 77.35 | 49.58 | 93.36 | 100.0 | o |
| SER | 60 | 76.55 | 53.42 | 39.24 | 37.31 | 48.2 | |
| GLY | 61 | 24.28 | 24.26 | 24.28 | 0.00 | 27.8 | |
| LEU | 62 | 26.19 | 26.19 | 0.00 | 26.19 | 17.9 | i |
| TYR | 63 | 41.62 | 10.54 | 0.00 | 41.62 | 21.6 | |
| SER | 64 | 12.35 | 9.95 | 0.65 | 11.70 | 15.1 | i |
| LEU | 65 | 14.66 | 14.66 | 8.18 | 6.48 | 4.4 | i |
| SER | 66 | 26.24 | 15.50 | 0.34 | 25.90 | 33.5 | |
| SER | 67 | 0.00 | 0.00 | 0.00 | 0.00 | 0.0 | i |
| VAL | 68 | 31.44 | 31.44 | 0.00 | 31.44 | 25.7 | |
| VAL | 69 | 0.00 | 0.00 | 0.00 | 0.00 | 0.0 | i |
| THR | 70 | 62.47 | 34.59 | 9.93 | 52.55 | 49.5 | |
| VAL | 71 | 5.98 | 5.98 | 2.71 | 3.28 | 2.7 | i |
| PRO | 72 | 81.01 | 81.01 | 0.51 | 80.50 | 76.5 | o |
| SER | 73 | 17.25 | 7.69 | 9.56 | 7.69 | 9.9 | i |
| SER | 74 | 81.09 | 63.02 | 20.39 | 60.70 | 78.4 | o |
| SER | 75 | 18.99 | 18.99 | 1.80 | 17.19 | 22.2 | |
| LEU | 76 | 48.87 | 30.22 | 18.85 | 30.02 | 20.5 | |
| GLY | 77 | 82.92 | 48.29 | 82.92 | 0.00 | 95.1 | o |
| THR | 78 | 126.95 | 88.03 | 38.10 | 88.85 | 83.7 | o |
| GLN | 79 | 80.67 | 21.24 | 4.68 | 75.99 | 52.9 | o |
| THR | 80 | 80.15 | 63.59 | 5.79 | 74.37 | 70.0 | o |
| TYR | 81 | 5.72 | 1.57 | 0.39 | 5.33 | 2.8 | i |
| ILE | 82 | 53.28 | 53.28 | 0.00 | 53.28 | 36.2 | |
| CYS | 83 | 0.00 | 0.00 | 0.00 | 0.00 | 0.0 | i |
| ASN | 84 | 13.67 | 8.31 | 1.67 | 12.00 | 10.5 | i |
| VAL | 85 | 0.00 | 0.00 | 0.00 | 0.00 | 0.0 | i |
| ASN | 86 | 24.22 | 6.17 | 0.34 | 23.87 | 20.9 | |
| HIS | 87 | 0.00 | 0.00 | 0.00 | 0.00 | 0.0 | i |
| LYS | 88 | 153.55 | 97.12 | 9.44 | 144.11 | 87.6 | o |
| PRO | 89 | 56.09 | 36.24 | 34.61 | 21.48 | 20.4 | |
| SER | 90 | 33.23 | 14.05 | 24.77 | 8.46 | 10.9 | i |
| ASN | 91 | 143.48 | 40.64 | 29.16 | 114.32 | 100.0 | o |
| THR | 92 | 30.70 | 17.39 | 5.76 | 24.94 | 23.5 | |
| LYS | 93 | 148.72 | 78.74 | 25.22 | 123.51 | 75.1 | o |
| VAL | 94 | 38.30 | 38.30 | 5.13 | 33.17 | 27.1 | |
| ASP | 95 | 82.49 | 24.32 | 21.15 | 61.34 | 54.3 | o |
| LYS | 96 | 77.53 | 28.82 | 0.58 | 76.96 | 46.8 | |
| ARG | 97 | 122.18 | 58.53 | 1.05 | 120.33 | 61.6 | o |
| VAL | 98 | 0.28 | 0.00 | 0.28 | 0.00 | 0.0 | i |
| GLU | 99 | 131.88 | 49.33 | 13.14 | 118.75 | 84.1 | o |

CH1 sequence and modeling information. Positions are indicated with an arbitrary number. Residue number 1 corresponds to EU-number 118, residue number 2 corresponds to EU-number 119 etc. Column In/Out indicates whether the amino acid is considered to be buried (i) or surface-exposed (o). An open space indicates a value for an amino acid that is not surface-exposed but also not buried.

Listed below is the amino acid sequence of the human CH1 region modelled in accordance with EU numbering, with underlined and italic amino acids representing non-surface exposed amino acid positions, and those in bold further representing amino acids that are buried.

TABLE 1b

| | |
|---|---|
| 118 | [symbol] S |
| 120 | T K [symbol] [symbol] S [symbol] F P L A. |
| 130 | [symbol] S S K [symbol] T S G G T |
| 140 | [symbol] A [symbol] [symbol] [symbol] L [symbol] K D [symbol] |
| 150 | F P E P [symbol] T [symbol] S [symbol] [symbol] |
| 160 | S [symbol] A L T S G [symbol] H T |
| 170 | F P [symbol] V L Q S S G [symbol] |
| 180 | Y [symbol] [symbol] S [symbol] V [symbol] T [symbol] P |
| 190 | [symbol] S S L G T Q T [symbol] I |
| 200 | [symbol] [symbol] [symbol] N [symbol] K P S N T |
| 210 | K V D K R [symbol] |

Rosetta software (version 3.1 https://www.rosettacommons.org/software) was used (in design mode) to model variation of non-surface residues in conjunction with in silico stability analysis, evaluating the impact of variations at these positions and impact on the stability of the protein. Rosetta design runs led to the predictions that the following variations at residues with <20% SASA in the starting model would improve stability: A172P, S190A, Y149A, V154I. Rosetta also predicted the following variations would improve stability: G122P, S157T, I199V, N203I, S207T, and V211I. After making the design variations from the first round of identified non-surface residues, two additional Rosetta designs were carried out: 1) where non-surface residues were only allowed to be varied if they increased the predicted positive charge (changing a residue to a positive charge or removing D or E), and 2) where residues were only allowed to be varied if they increased the predicted negative charge (to D or E from neutral, or from K and R to non-charged).

Buried residues N159 (N42) and N201 (N84) were found to sustain variations of a positive charge (K) and negative charge (D) residue, while maintaining good stability. Other non-surface exposed residues were identified with potential to support charge variations without significant predicted detraction of the stability of CH1, and including certain variations predicted to improve the stability according to the bioinformatics analysis (with the more negative number score meaning more stable).

TABLE 2
WT stability score of −632.956

1. [symbol]
2. [symbol]
3. [symbol]
4. [symbol]
5. [symbol]
6. [symbol]
7. [symbol]
8. [symbol]
9. [symbol]
10. [symbol]
11. [symbol]
12. [symbol]
13. [symbol]
14. [symbol]
15. [symbol]
16. [symbol]
17. [symbol]
18. [symbol]
19. [symbol]
20. [symbol]
21. [symbol]
22. [symbol]
23. [symbol]
24. [symbol] [symbol]
indicates data missing or illegible when filed

### Example 1 b: Construct Design

Non-surface and buried positions in CH1 are varied to change the charge of multimerizing proteins incorporating these immunoglobulin regions. In total 13 exemplary variant CH1 regions are produced and incorporated into mono, and multispecific antibodies for comparison against mono, and multispecific antibodies with wild type CH1 regions. Constructs to express these molecules comprising these separation CH1 regions are prepared as follows.

The fragment encoding the CH2 and CH3 domain was obtained from the MV1708 construct. MV1708 was chosen as it contains a unique BspEI site at the N terminus of CH2. The fragment encoding the variable heavy chain MF1122 was used, which has a BstEII on its C-terminus. MF1122 was chosen because it does not present any issues with production, purification or CIEX and has an average retention time on CIEX of ˜13.4 min at a pI (VH) of 8.64. The constructs used for cloning and the cloning strategy are displayed in FIG. 12.

Vector MV1708 (containing DE variations in CH3) was modified to contain a WT CH3 region. The VH gene from MF1122 was inserted in the vector using Sfil and BstEll-HF restriction enzymes. Correct colonies are selected by colony PCR and sequencing.

In the construct, the sequence encoding for the CH1 region is flanked by the restriction sites BstEII and BspEI. This allows exchange of the CH1 encoding sequence. Plasmids containing the wild type or variant CH1 regions were produced. The specific sequences for each variant CH1 region are listed below.

The CH1 encoding sequences (363 bp) were excised from the plasmids using BstEll and BspEI (2 ug of each of CH1 encoding constructs). Simultaneously, the prepared vector was excised from the plasmids using BstEll and BspEI restriction enzymes (20 ug of the vector). Plasmids are incubated for at least 1 h with BspEI (0.25 uL enzyme/ug DNA) in buffer NEBuffer3.1 at 37 degrees, followed by heating of the mixture to 60 degree and addition of BstEll. Digested DNA was purified by Gel Electrophoresis and Gel Extraction. The digest removes a 748 bp fragment from the backbone (˜10 kb) and 363 bp fragment from the CH1 domain and hinge-containing constructs.

Ligation of the vector with the CH1 coding sequence was carried out followed by transforming into DH5a cells and plated on 113 agar plates containing Ampicillin. The correct constructs are identified by colony PCR and sequencing permitting identification of the correct CH1 and correct, CH2-CH3. The identity of the final constructs was confirmed by sequencing.

### Examples 1 c: Expression and Purification of Antibodies with CH1 Variants

All used buffers were made using Versylene (endotoxin-free and sterile) water. Endotoxin was removed from glasswork. Quixstand. Akta-explorer by incubation with 0.1M NaOH for at least 16 hours. Hek293 cells were transfected with endotoxin-free plasmid DNA. Six days post-transfection conditioned medium containing recombinant antibody was harvested by low-speed centrifugation (10 minutes, 1000 g) followed by high-speed centrifugation (10 minutes, 4000 g). A 100 ul sample was stored at 4° C.

MabSelectSureLX (GE healthcare life sciences) purification was performed: The antibody was bound batch-wise to 2 ml MabSelectSureLX for 4-hours. MabSelectSureLX sepharose containing bound antibody was harvested by centrifugation and transferred into gravity flow column. Non-specifically bound proteins were removed by washing the column with PBS, PBS containing 1 M NaCl and PBS. The bound antibody was eluted using 100 nM citrate pH 3.5 and 5 ml fractions were collected in 12 ml tubes containing 4 ml 1 M Tris pH 8.0 for neutralization to pH 7. Protein containing fractions were pooled. The MabSelectSureLX pool was concentrated to 2.0-3.0 ml using vivaspin20 10 kDa Spin filter. Aggregates in the concentrated pool were removed by centrifugation. The concentrated sample was stored at 4° C. before gelfiltration.

Gelfiltratation: the recombinant antibody was purified further by gelfiltration using a superdex 200 16/600 column, which was equilibrated in PBS. Protein containing fractions were analyzed by LabChip (PerkinElmer) and correct antibody containing fractions were pooled. The pool was sterilized by filtration using a 0.22 um syringe filter. The product was stored in aliquots containing 1.8 ml at 4° C. The product was analyzed by LabChip capillary electrophoresis (PerkinElmer) and LAL assay (Endotoxin assay).

The LabChip analysis was performed under reducing and non-reducing conditions. HP-SEC analysis of the samples showed only one major peak for the antibodies, indicating that the samples do not contain aggregates or half-bodies.

### Example 1d: Generation of Constructs to Produce CH-1 Modified Bispecific Antibodies

Exchange of the DE Arm of the Heavy Chain with a KK Arm

A second vector encoding a heavy chain was produced. The heavy chain encoded by this vector comprises a KK arm in order to distinguish the heavy chains from the heavy chain with a DE arm. Production of 2 different heavy chains allows the preferential formation of bispecific antibodies. The vector encoding the KK heavy chain was produced as follows.

The fragment encoding for the DE arm of the antibody was exchanged for a fragment encoding for a KK arm. The arms are exchanged using the flanking restriction sites BspEI and AflII in the construct and using cloning techniques as described above. Subsequently, the DE heavy chain was combined with a heavy chain variable domain VH region MF1516 and the KK heavy chain was combined with a heavy chain variable domain VH region MF3462. This cloning step was performed using the restriction enzymes SfiI and BstEII and cloning techniques as described above. The identity of the final constructs was confirmed by sequencing.

This cloning procedure results in vectors encoding for two heavy chains having a different binding specificity. When expressed together the heavy chains preferentially form bispecific antibodies. The CH1 variants can be inserted in each of the 2 heavy chains by applying the cloning steps as described above.

### Example 2: Demonstrating Capacity to Separate Identical Monospecific Antibodies Based on pI Separation Residues in the CH1 Separation Domain

In order to express the antibodies, a combination of nucleic acid constructs is used. The constructs encode for a common light chain (FIG. 13A and a heavy chain comprising a heavy chain variable region targeting fibrinogen (MF1122) (set out below). The heavy chain further comprises a (CH1 separation domain having a negative charge difference or a positive charge difference as compared to a wild-type human CH1. Expression of the constructs preferentially leads to formation of a monospecific IgG1 human antibody. The rearranged germline human kappa light chain IgVκ1-39*01/IGJκ1*01 is used as common light chain.

TABLE 3
Sequences of the light chain

| cLC sequences | Sequences |
|---|---|
| Amino acid- | DIQMTQSPSSLSASVGDRVTITCRASQSISSY |
| sequence of | LNWYQQKPGKAPKLLIYAASSLQSGVTSRFSG |
| the common | SGSGTDFTLTISSLQPEDFATYYCQQSYSTPP |
| light chain | PTFGQGTKVEIK |
| variable | (SEQ ID NO: 1) |
| region | |
| IgV_{K}1- | |
| 39*01/ | |
| IGJ_{K}1*01 | |
| Amino acid- | RTVAAPSVFIFPPSDEQLKSGTASVVCLLNNF |
| sequence | YPREAKVQWKVDNALQSGNSQESVTEQDSKDS |
| light chain | TYSLSSTLTDSKADYEKHKVYACEVTHQGLSS |
| constant | PVTKSFNRGEC |
| region (CL) | (SEQ ID NO: 2) |
| DNA sequence: | GACATCCAGATGACCCAGTCTCCATCCTCCCT |
| of IgV_{K}l- | GTCTGCATCTGTAGGAGACAGAGTCACCATGA |
| 39*01/ | CTTGCCGGGCAAGTCAGAGCATTAGCAGCTAC |
| IGJ_{K}1*01 | TTAAATTGGTATCAGGAGAAACCAGGGAAAGC |
| | CCCTAAGCTCCTGATCTATGCTGCATCCAGTT |
| | TGCAAAGTGGGGTCCCATCAAGGTTCACTGGC |
| | AGTGGATCTGGGACAGATTTCACTCTCACCAT |
| | CAGCAGTCTGCAACCTGAAGATTTTGCAACTT |
| | ACTACTGTCAACAGAGTTACAGTACCCCTCCA |
| | ACGTTCGGCCAAGGGACCAAGGTGGAGATCAA |
| | A(SEQ ID NO: 3) |
| DNA sequence: | CGAACTGTGGCTGCACCATCTGTCTTCATCTT |
| light chain | CCCGCCATCTGATGAGCAGTTGAAATCTGGAA |
| constant | CTGCCTCTGTTGTGTGCCTGCTGAATAACTTC |
| region (CL) | TATCCCAGAGAGGCCAAAGTACAGTGGAAGGT |
| | GGATAAGGCGCTCCAATCGGGTAACTCCCAGG |
| | AGAGTGTCACAGAGCAGGACAGCAAGGACAGC |
| | ACCTACAGCCTCAGCAGCACCGTGACGCTGAG |
| | CAAAGCAGACTACGAGAAACACAAAGTCTACG |
| | CCTGCGAAGTCACCCATCAGGGCCTGAGCTCG |
| | CCCGTCACAAAGAGCTTCAACAGGGGAGAGTG |
| | T(SEQ ID NO: 4) |

The amino acid sequences of the heavy chain variable region capable of binding fibrinogen (MF1122) that was used in these experiments is listed below. The CH1, CH2 and CH3 regions are human IgG1 (FIGS. 14A-14E).

The target of the heavy chain variable domain is fibronectin, the isoelectric point of the heavy chain variable domain is 8.64 (pI) and the isoelectric point of the full heavy chain is 8.54 (pI).

TABLE 4
Amino acid sequences of the various parts of
the heavy chain variable region of MF1122
capable of binding fibrinogen. Further
described is a heavy chain variable
region capable of binding PD-L1 (VH PD-L1).
The variable domain can combine in a
variable domain with the common light
chain. The heavy chain variable region of
MF1122 has a pI of 8.64. The heavy chain
variable region that targets PD-L1 has
a pI of 5.73.

| | MF1122 heavy chain variable region |
|---|---|
| | amino acid sequences |
| FW1 | EVQLVESGGGVVQPGRSLRLSCAASGETES |
| | (SEQ ID NO: 5) |
| CDR1 | SYGMH (SEQ ID NO: 6) |
| FW2 | WVRQAPGKGLEWVA (SEQ ID NO: 7) |
| CDR2 | VISYDGSNKYYADSVKG (SEQ ID NO: 8) |
| FW3 | RFTISRDNSKNTLYLQMNSLRAEDTAVYYCAR |
| | (SEQ ID NO: 9) |
| CDR3 | ALFTTIAMDY (SEQ ID NO: 10) |
| FW4 | WGQGTLVT (SEQ ID NO: 11) |
| VH | EVQLVESGGGVVQPGRSLRLSCAASGFTFSSYGMHWVRQ |
| | APGKGLEWVAVISYDGSNKYYADSVKGRFTISRDNSKNT |
| | LYLQMNSLRAEDTAVYYCARALFTTIAMDYWGQGTLVTV |
| | SS (SEQ ID NO: 12) |

The following CH1 variants tested are provided below, with residue variations identified in accordance with EU numbering.

TABLE 5

| | Charge difference of |
|---|---|
| | antibodies |
| | comprising two |
| | variant CH1 regions |
| Variant Positions by EU- | relative to wild-type |
| numbering | CH1 |
| T197D K213Q | −4 |
| K213Q N159D | −4 |
| K147E Q175E | −6 |
| N201D K213Q | −4 |
| K213Q | −2 |
| N201D | −2 |
| N201D A172P S190A | −2 |
| wt | 0 |
| Y149A, V154I, A172P S190A | 0 |
| T120K | +2 |
| N201K | +2 |
| N201K A172P S190A | +2 |
| D148K Q175K | +6 |
| N159K E216K(hinge) | +6 |

Sufficient and similar amounts of each antibody were produced with volume yields in the range of 10-25 ml having concentrations of about 1.7 mg/mL

For each antibody the CIEX retention time was determined.

CIEX-HPLC chromatography was done using TSKgel SP-STAT (7 μm particle size, 4.6 mM I.D.×10 cm L, Tosoh 21964) series of ion exchange columns. The CIEX assay uses a hydrophilic polymer based column material packed with non-porous resin particles, of which the surface consists of an open access network of multi-layered cation exchange groups (sulfonic acid group), making it a strong cation exchanger and therefore suitable for separation of charge isomers of monoclonal antibodies by using NaCl salt gradients. Positively charged antibodies will bind to the negatively charged column.

The TSKgel SP-STAT (7 μm particle size, 4.6 mM I.D×10 cm L, Tosoh 21964) is equilibrated using Buffer A (Sodium Phosphate buffer, 25 mM, pH 6.0) for at least 30 min at ˜50 bars pressure. This is followed by injection of control and sample IgGs. The injection sample mass for all test samples and controls (in PBS) was 10 μg protein and injection volumes 10-100 μl. The antibodies are displaced from the column by increasing salt concentration and running a gradient of Buffer B (25 mM Sodium Phosphate, 1 mM NaCl, pH 6.0). Flow rate was set at 0.5 mL/min. The chromatograms were analysed for peak patterns, retention times and peak areas for the major peaks observed based on the 220 nm results.

In this study, the retention time correlated with the total charge difference as compared with wild-type, i.e. the more positive charge added, the longer the retention time, and the more negative charge added, the shorter the retention time.

TABLE 6
CIEX retention time of monospecific antibodies with
CH1 variants

| | | Experiment 1 | |
|---|---|---|---|
| | | FIGS. 11A-11E | |
| | | | diff. RT |
| variations | total charge | RT on | from |
| (EU) | diff from wt | CIEX | WT |
| T197D K213Q | −4 | 11.7 | −1.7 |
| K213Q N159D | −4 | 12.1 | −1.3 |
| K147E Q175E | −6 | 12.1 | −1.3 |
| N201D K213Q | −4 | 12.2 | −1.2 |
| K213Q | −2 | 12.4 | −1 |
| N201D | −2 | 13.2 | −0.2 |
| N201D A172P | −2 | 13.3 | −0.1 |
| S190A | | | |
| WT (none) | 0 | 13.4 | 0 |
| A172P S190A | 0 | 13.5 | 0.1 |
| Y149A V154I | | | |
| T120K | 2 | 15.6 | 2.2 |
| N201K | 2 | 15.7 | 2.3 |
| N201K A172P | 2 | 15.8 | 2.4 |
| S190A | | | |
| D148K, Q175K, | 6 | 17.7 | 4.3 |
| N159K | | | |
| E216K(hinge) | 6 | 21 | 7.6 |

The CIEX retention time for all CH1 variants is displayed in table 6 and FIGS. 11A-11E. These data demonstrate that antibodies otherwise having identical pI—for example, the bivalent, monospecific human IgG antibodies as described above, comprising a CH1 separation domain for each heavy chain and comprising wild-type human CH2 and CH3 domains and a common light chain—can be adequately separated based solely on use of the separation residues provided above, such that retention differentials of 0.1 to 7.6 are generated relative to wild-type CH1 region, by incorporation of one or more positive or negative charge difference residues per CH1 separation domain.

### Example 3: Stability Analysis of Antibodies Incorporating the Separation Residues Demonstrating Suitable Stability for Development

Stability of bivalent, monospecific antibodies in PBS was determined by freezing and thawing the antibodies which indicated that all bivalent, monospecific antibodies had comparable stability as the wild-type monoclonal antibody.

The composition of the samples was analysed after 1 freeze/thaw cycle with HP-SEC. Samples were stored at −80° C. overnight and the following day thawed at room temperature. For each antibody 21 μg dissolved in PBS was analysed with HP-SEC. All antibodies eluted as 1 major peak, indicating that the produced antibodies are stable after a freeze-thaw cycle. Accordingly, the samples maintain their composition when stored at −80° C.

Furthermore, the antibodies incorporating the separation domains were evaluated by determining temperature melting curves using DSC (Differential Scanning Calorimetry). To perform DSC, the antibody was diluted until 0.5 mg/mL in PBS, and dialyzed in dialysis buffer. The antibodies were subsequently filtered through a 0.45 um filter. After dialysis the samples were diluted to a concentration of 0.25 mg/mL to perform DCS analysis and obtain a temperature melting curve for each antibody. Temperature melting (TM) curves are depicted in FIG. 3. The TM1 and TM2 as determined from the temperature melting curve are listed in Table 7 below (DSC). In a second stability assay, TM2 was determined using UNcle (Unchained Labs), as set out in Table 8. The results are listed in the tables below. A ranking was also provided ranking the stability of IgGs with regard to TM2. The samples were heated from 25 degrees to 95 degrees Celsius at 0.5 degrees/min, in PBS buffer and tested at a pH of 7.4, with protein samples ranging from 0.2-1 mg/ml. The Tm/Tagg temperatures were then computed from fluorescence signal, and performed in triplicate.

UNCLE (Unchained Labs) was used to perform thermal stability studies by Differential Scanning Fluorometry (DSF) and Static Light Scattering (SLS). DSF is based on the detection of intrinsic amino acid fluorescence between 250 and 720 nm and used to infer protein unfolding upon denaturation. SIS detects changes in aggregate content by changes of light scattering of a laser with 266 nm. In brief, proteins are analyzed at 50 ug/mL and subjected to an increase of temperature from 25 to 95 degree (0.3 or 0.5 degree/minute). Heat denaturation induces changes of protein fluorescence (monitored between 250 and 720 nm) and light scattering (light of a laser with 266 nm) which are detected and analyzed. Changes in fluorescence are displayed as BCM (barycentric mean: the detected fluorescence spectrum is divided in two equal areas) over temperature. The UNCLE analysis software is used to calculate the differential of the change of fluorescence over temperature graph and identify the presence of melting points (TM—temperature at which a change in fluorescence occurs) and temperature-induced aggregation (TAGG—temperature at which the signal of static light scatter at 266 nm increases by about 10% above baseline).

TABLE 7
DSC analysis of the Temperature melting (TM) curves result in a TM1, and
TM2. Temperature melting curves are depicted in FIG. 3.

| DSC | | |
|---|---|---|
| CH1 version | | |
| (EU) | TM1 | TM2 |
| Wt | 71.9 | 84.8 |
| T197D, K213Q | 70.6 | 84.7 |
| K213Q, N159D | 71.7 | 82.1 |
| K147E, Q175E | 70.6 | 79.0 |
| N201D, K213Q | 70.7 | 82.0 |
| K213Q | 70.7 | 84.8 |
| T120K | 71.6 | 83.6 |
| N201K | 70.6 | 83.7 |
| N201K, A172P, S190A | 70.6 | 83.4 |
| D148K, Q175K | 69.2 | 70.1 |
| N159K, | 70.7 | 81.7 |
| E216K(hinge) | | |

TABLE 8
Stability of the antibody variants was measured using UNCLE
and DSC. Agg refers to aggregation occurring
before melting. ND indicates no data (these samples
have not been analysed with DSC).

| | CH1 variations | UNCLE | DSC |
|---|---|---|---|
| Ranking | (EU) | TM2 | TM2 |
| 1 | wt (none) | 85.5 | 84.8 |
| 2 | T197D, K213Q | 85.5 | 84.7 |
| 3 | K213Q | 85 | 84.8 |
| 4 | T120K | 84 | 83.6 |
| 5 | K213Q, N159D | 83.9 | 82.1 |
| 6 | N201D, K213Q | 83.4 | 82 |
| 7 | N201K | 81.8 | 83.7 |
| 8 | N201K, A172P, S190A | 81 | 83.4 |
| 9 | N201D | 80.8 | ND |
| 10 | N201D, A172P, S190A | 80.2 | ND |
| 11 | N159K, E216K(hinge) | 79.4 | 81.7 |
| 12 | K147E, Q175E | agg | 79 |
| 13 | D148K, Q175K | agg | 70.1 |
| 14 | A172P S190A Y149A | agg | ND |
| | V154I | | |

### Example 4: Isoelectric Focusing

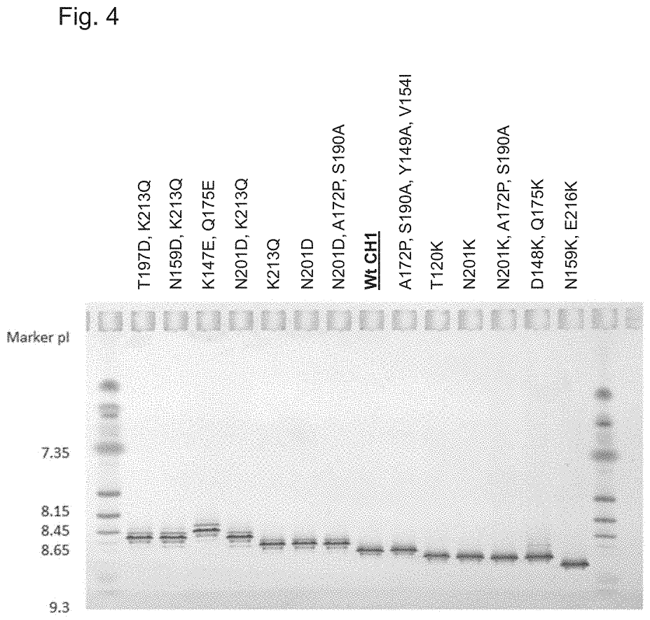

When produced. IgG was run on SDS-page gels, under reduced and non-reduced conditions. All protein sizes were as expected and all bands for each variant were at the same height. In addition, IgGs produced were run on gels using iso-electric focusing, the results thereof are depicted in FIG. 4. The relative migration of bands on gel correlated with a calculated pI listed below (FIG. 4).

TABLE 9
Relative migration of bands on SDS-page gel
correlated with a calculated pI

| | | theoretical |
|---|---|---|
| | | Full Heavy |
| | | Chain |
| | total charge | Polypeptide |
| CH1 variations (EU) | diff from wt | pI |
| T197D K213Q | −4 | 8.36 |
| K213Q N159D | −4 | 8.36 |
| K147E Q175E | −6 | 8.19 |
| N201D K213Q | −4 | 8.36 |
| K213Q | −2 | 8.49 |
| N201D | −2 | 8.49 |
| N201D A172P S190A | −2 | 8.49 |
| wild-type (none) | 0 | 8.60 |
| A172P S190A Y149A | 0 | 8.60 |
| V154I | | |
| T120K | 2 | 8.69 |
| N201K | 2 | 8.69 |
| N201K A172P S190A | 2 | 8.69 |
| D148K Q175K | 6 | 8.85 |
| N159K E216K(hinge) | 6 | 8.85 |

### Example 5: Separation of Bispecific and Monospecific Antibodies by Use of the CH1 Separation Domains (Comprising Separation Residues)

Bispecific antibodies are produced by expressing 2 different heavy chains together. In order to form antibodies these heavy chains were paired with the common light chain as described above.

The experiments are performed with a heavy chain having a DE arm and a heavy chain having a KK arm. The cloning of these constructs is described in example 1d. The DE or KK modification is located in the CH3 domain of the heavy chain.

Each heavy chain consists of a CH3, CH2, CH1 and VH domain. The CH3 domain allows heterodimerization of the heavy chain antibodies and contains either the DE or KK residues for the 2 different heavy chains. The CH2 domain is a human CH2 domain. The VH determines the specificity of the antibody, whereby the DE heavy chain targets tetanus toxin (T) (MF1516) and the KK heavy chain targets cMet (MF3462). Sequences provided below at Tables 10 and 11.

The CH1 regions of the heavy chains are either wild type or a separation domain as described herein producing a charge differential from the wild type domain. The heavy chain having a DE arm is a variant of the human wild type CH3 domain for promoting heterodimerization. The heavy chain having a KK arm is a variant of the human wild type CH3 for promoting heterodimerization. The DE arm is linked to a separation domain having a negative charge differential as compared to a wild type domain and the KK arm is linked to a separation arm having a positive charge differential as compared to a wild type domain.

TABLE 10
Amino acid sequences of the various parts of
the DE heavy chain variable region. The heavy
chain targets tetanus toxin (ME1516). The heavy
chain variable region has a pI of 8.64 and the
full heavy chain has a pI. of 8.54.

| | |
|---|---|
| FW1 | EVQLVETGGGWQPGRSLRLSCAASGFTFS |
| | (SEQ ID NO: 13) |
| CDR1 | QYAMH(SEQ ID NO: 14) |
| FW2 | WVRQAPGKGLEWVA (SEQ ID NO: 15) |
| CDR2 | IISHDERNKYYVDSGMG (SEQ ID NO: 16) |
| FW3 | RFTISRDNSKNTLFLQMNSLRSEDTAVYYCAR |
| | (SEQ ID NO: 17) |
| CDR3 | DMRKGGYYYGFDV (SEQ ID NO: 18) |
| PW4 | WGQGTTVT (SEQ ID NO: 19) |
| VH | EVQLVBTGGGWQPGRSLRLSCAASGFTFSQYAMHWVRQAP |
| | GKGLEWVAIISHDERNKYYVDSGMGRFTISRDNSKNTLFL |
| | QMMSLRSEDTAVYYCARDMRKGGYYYGFDWGQGTTVTVSS |
| | (SEQ ID NO: 20) |

TABLE 11
Amino acid sequences of the various parts
of the KK heavy chain variable region and the
light chain variable region. The heavy chain
that targets cMet (MF3462) has a pI of 8.04
and the full heavy chain has a pI of 8.46.

| | |
|---|---|
| FW1 | EVQLLESGGGLVQPGGSLRLSCAASGFTFS |
| | (SEQ ID NO: 21) |
| CDR1 | SYAMS (SEQ ID NO: 22) |
| FW2 | WVRQAPGKGLEWVS (SEQ ID NO: 23) |
| CDR2 | AISGSGGSTYYADSVKG (SEQ ID NO: 24) |
| FW3 | RFTISRDNSKNTLYLQMNSLRAEDTAVYYCAR |
| | (SEQ ID NO: 25) |
| CDR3 | GKSHYSWDAFDY(SEQ ID MO: 26) |
| FW4 | WGQGTLVTVSS(SEQ ID NO: 27) |
| VH | EVQLLESGGGLVQPGGSLRLSCAASGFTFSSYAMSW |
| | VRQAPGKGLEWVSAISGSGGSTYYADSVKGRFTISR |
| | DNSKNTLYLQMNSLRAEDTAVYYCARGKSHYSWDAF |
| | DYWGQGTLVTVSS |
| | (SEQ ID NO: 28) |

Amino acid sequences of antibodies that target Tetanus Toxoid identified with number have the amino acid sequence of MF1337

MF1337:
(SEQ ID NO: 29)
EVQLVETGAEVKKPGASVKVSOKASDYIFTKYDINW
VRQAPGQGLEWMGWMSANTGNIGYAQKFQGRVTMTR
DTSINTAYMELSSLTSGDTAVYFCARSSLFKTETAP
YYHFALDVWGQGTIVEVES

The bispecific antibodies were produced by transfecting IgG1 heavy chain constructs with alight chain construct into HEK293 cells as follows. Suspension adapted 293 cells were cultivated in T125 flasks at a shaker plateau until a density of 3.0×10\hat{ }6 cells/ml. Cells were seeded at a density of 0.3-0.5×10\hat{ }6 viable cells/ml in each well of a 24-deep well plate. The cells were transiently transfected with individual sterile DNA: PEI mixtures according to standardized procedures and further cultivated. Seven days after transfection, supernatant was harvested and filtered through a 0.22 μM filter. The sterile supernatant was stored at 4° C. until antibody was purified by means of protein-A affinity chromatography. Antibodies were subsequently expressed in HEK293 cells by transient transfection and purified from the culture supernatant using protein-A affinity chromatography according to standard procedures.

IgG Purification for Functional Screening Purification of IgG was performed on a small scale (<500 μg), medium scale (<10 mg) and large scale (>10 mg) using protein-A affinity chromatography. Small scale purifications were performed under sterile conditions in 24 well filter plates using filtration. First, the pH of the medium was adjusted to pH 8.0 and subsequently, IgG-containing supernatants were incubated with protein A Sepharose CL-4B beads (50% v/v) (Pierce) for 2 hrs at 25° C. on a shaking platform at 600 rpm. Next, the beads were harvested by filtration. Beads were washed twice with PBS pH 7.4. Bound IgG was then eluted at pH 3.0 with 0.1 M citrate buffer and the eluate was immediately neutralized using Tris pH 8.0. Buffer exchange was performed by centrifugation using multiscreen Ultracel 10 multiplates (Millipore). The samples were finally harvested in PBS pH 7.4. The IgG concentration was measured using Octet (ForteBio). Protein samples were stored at 4° C.

The following constructs were made and used in the experiments. Before conducting the experiments, constructs were validated with regard to sequence. The encoded heavy chains were produced and analysed using SDS-page under reduced and non-reduced conditions. All heavy chains produced bispecific monovalent antibodies and halfbodies of expected sizes.

TABLE 12
CH1 variations in the heavy chain
having either a DE or KK arm.

| variant (EU) | DE or KK |
|---|---|
| T197D K213Q | DE |
| K213Q | DE |
| T120K | KK |
| N201K | KK |
| N159K | KK |
| E216K(hinge) | |
| wt | DE |
| wt | KK |

The various DE heavy chains were combined with the various KK heavy chains in order to produce bispecific antibodies. The products are analysed with CIEX as described in example 2. Both the combinations of DE/KK antibodies as well as one arm productions with either DE or KK are analysed. One arm production with DE resulted in DE/DE homodimers and one arm productions with KK resulted in KK halfbodies. KK/KK homodimers were not observed to be produced.

The table below describes the retention times for the various antibody species and the one arm productions. The relative difference in retention time between the bispecific antibody (DE/KK) and the homodimer (DE/DE) or the KK half body indicates the distance between the peaks in the CIEX spectrum. A larger difference makes it easier to separate the fractions with the bispecific antibody form the homodimers and halfbodies.

TABLE 13
CIEX retention times of bispecific antibodies with CH1 separation
domains. Retention time (RT), relative differences (ΔRT)

| | | | | | ΔRT | ΔRT |
|---|---|---|---|---|---|---|
| CH1/DE | CH1/KK | | | | (DEKK − | (DEKK − |
| arm | arm | bispecific | DEDE | KK half | DEDE | KK half |
| (EU) | (EU) | (DEKK) | homodimer | body | homodimer) | body) |
| wt | wt | 15.5 | 13.9 | 19.3 | 1.6 | 3.8 |
| wt | T120K | 15.8 | 13.8 | 19.9 | 2 | 4.1 |
| wt | N201K | 16.2 | 13.6 | 20.5 | 2.6 | 4.3 |
| wt | N159K | 18.7 | 13.6 | 23.2 | 5.1 | 4.5 |
| | E216K(hinge) | | | | | |
| T197D | wt | 14.3 | 12 | 18.9 | 2.3 | 4.6 |
| K213Q | | | | | | |
| T197D | T120K | 14.9 | 12 | 19.7 | 2.9 | 4.8 |
| K213Q | | | | | | |
| T197D | N201K | 15.4 | 12 | 20.5 | 3.4 | 5.1 |
| K213Q | | | | | | |
| T197D | N159K | 18 | 12 | 23.2 | 6 | 5.2 |
| K213Q | E216K(hinge) | | | | | |
| K213Q | wt | 14.7 | 12.7 | 18.9 | 2 | 4.2 |
| K213Q | T120K | 15.2 | 12.7 | 19.7 | 2.5 | 4.5 |
| K213Q | N201K | 15.7 | 12.7 | 20.5 | 3 | 4.8 |
| K213Q | N159K | 18.2 | 12.7 | 23.2 | 5.5 | 5 |
| | E216K(hinge) | | | | | |
| wt | — | NA | 13.6 | NA | NA | NA |
| T197D | — | NA | 12 | NA | NA | NA |
| K213Q | | | | | | |
| K213Q | — | NA | 12.7 | NA | NA | NA |
| — | wt | NA | NA | 18.9 | NA | NA |
| — | T120K | NA | NA | 19.7 | NA | NA |
| — | N201K | NA | NA | 20.5 | NA | NA |
| | N159K | NA | NA | 23.2 | | |
| | E216K(hinge) | | | | | |

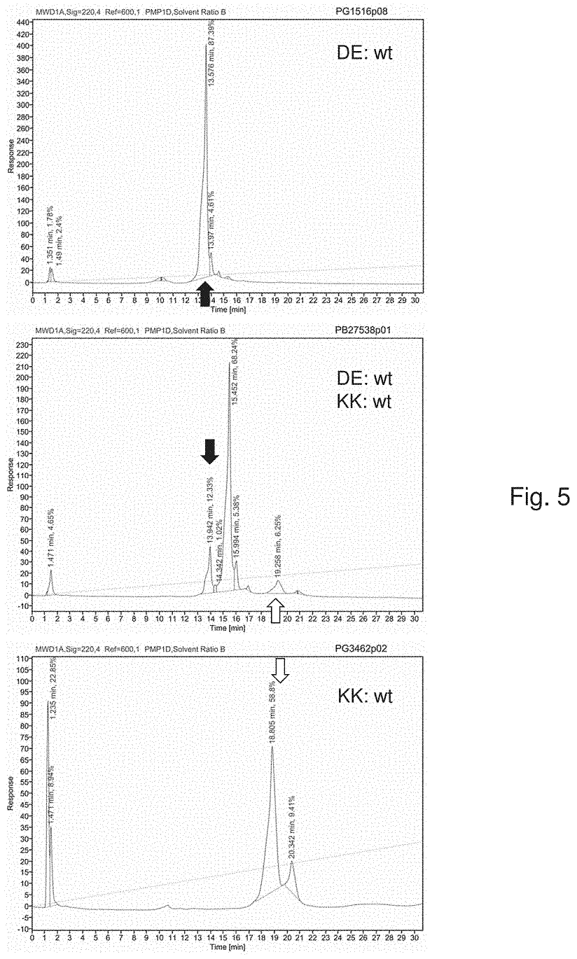

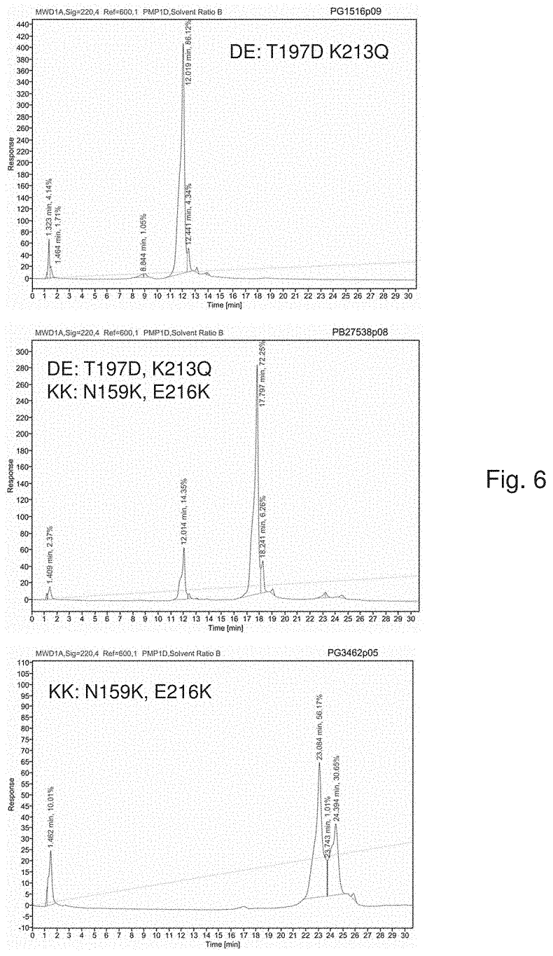

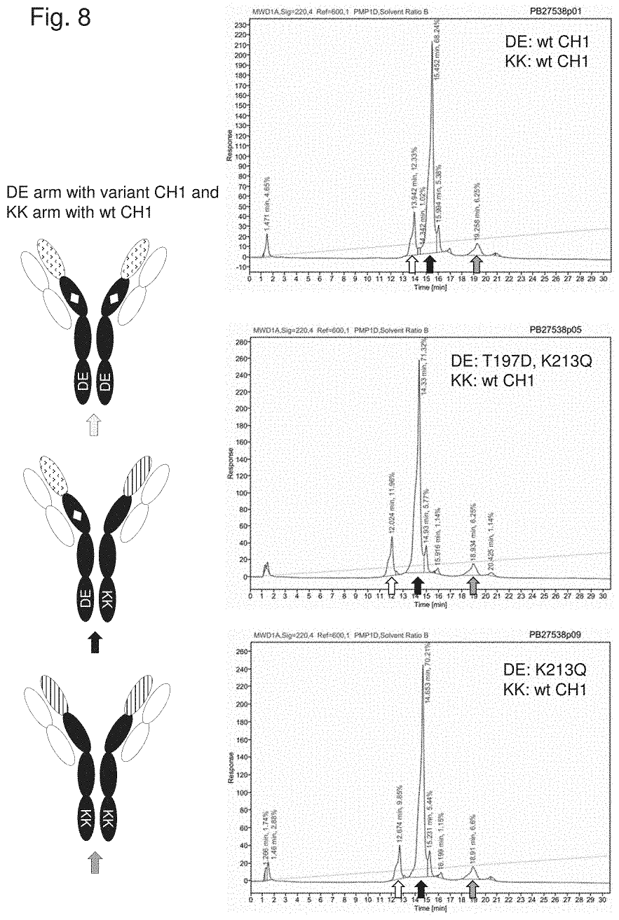

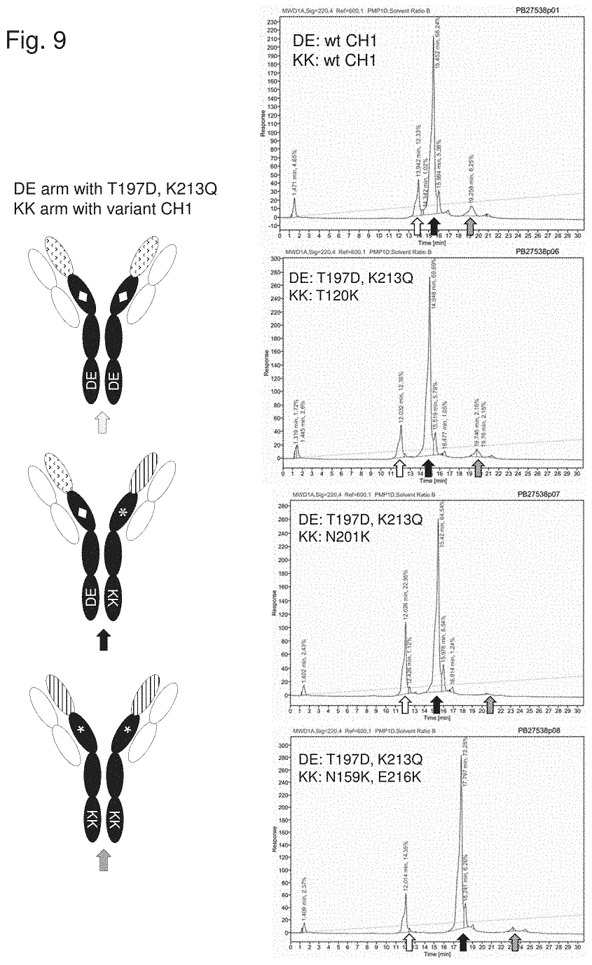

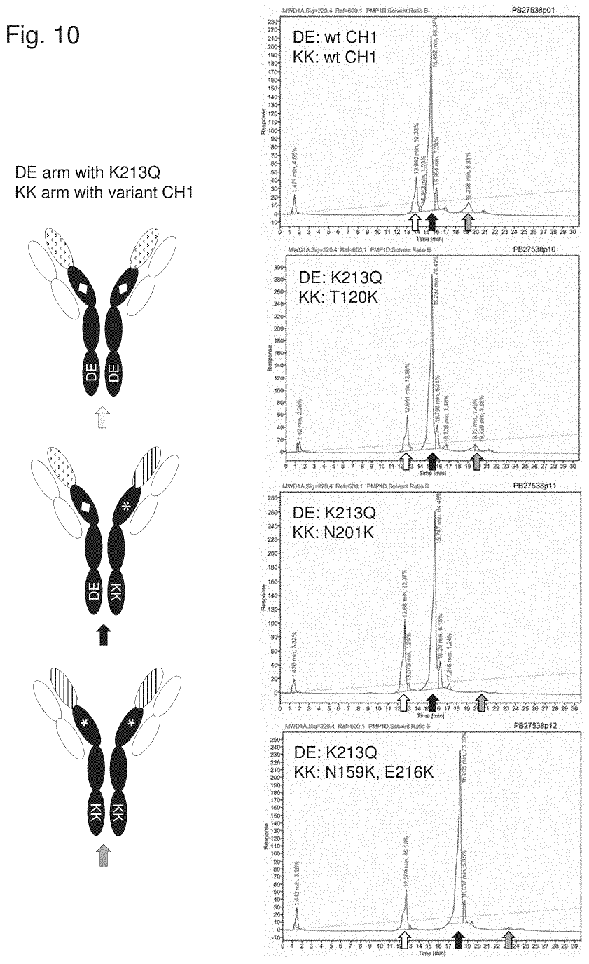

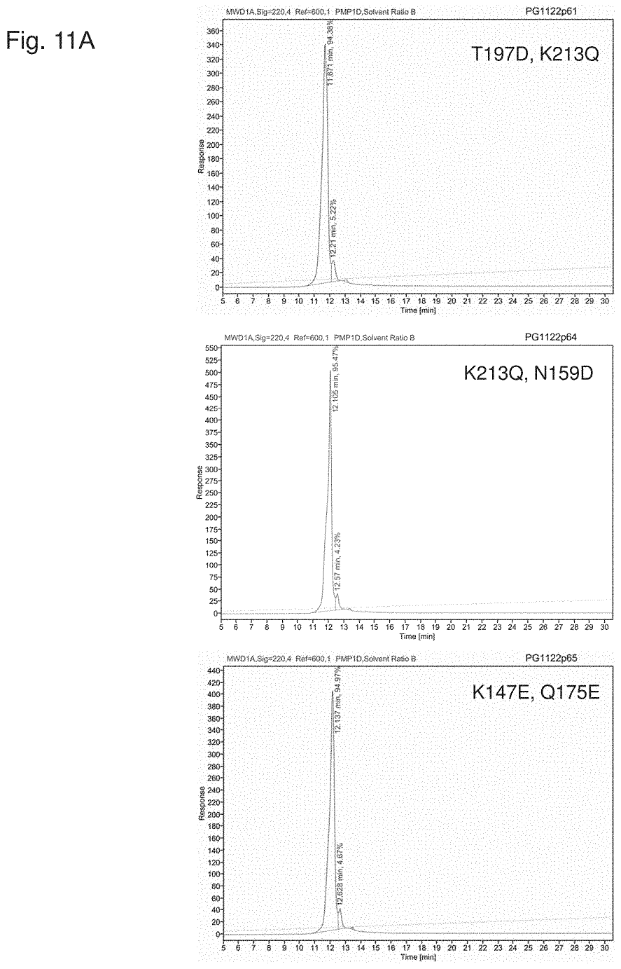

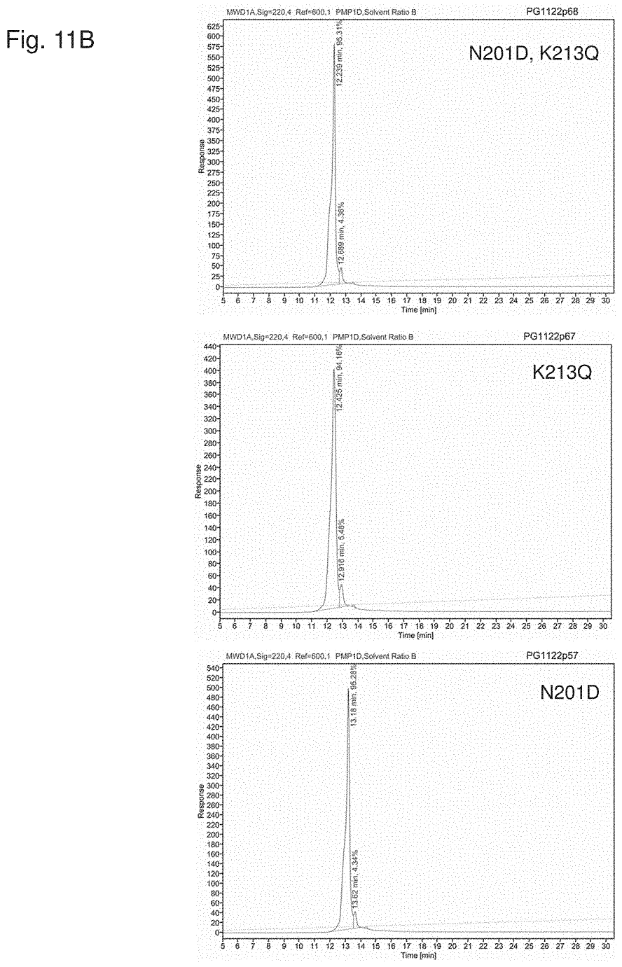

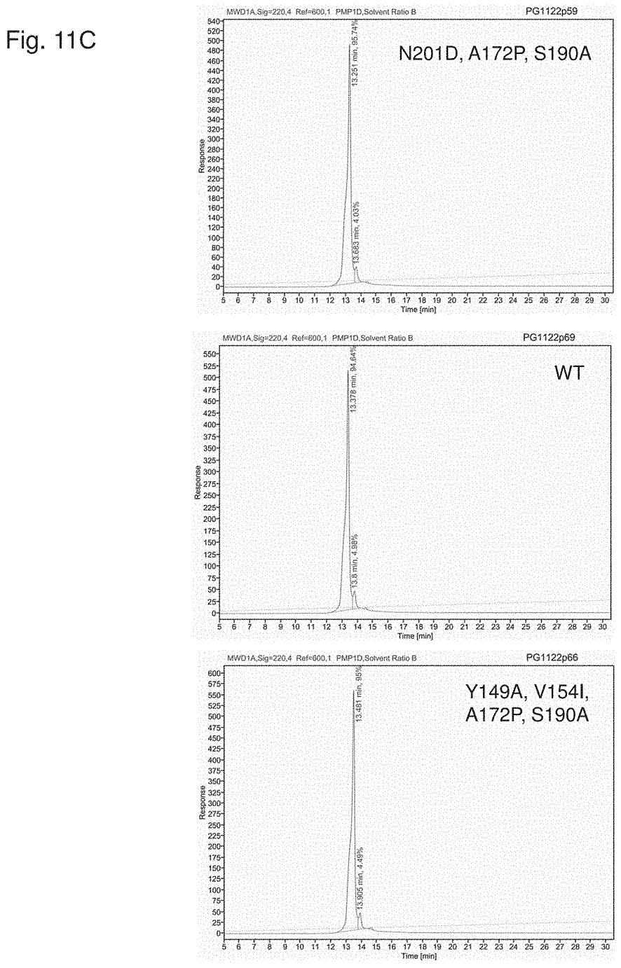

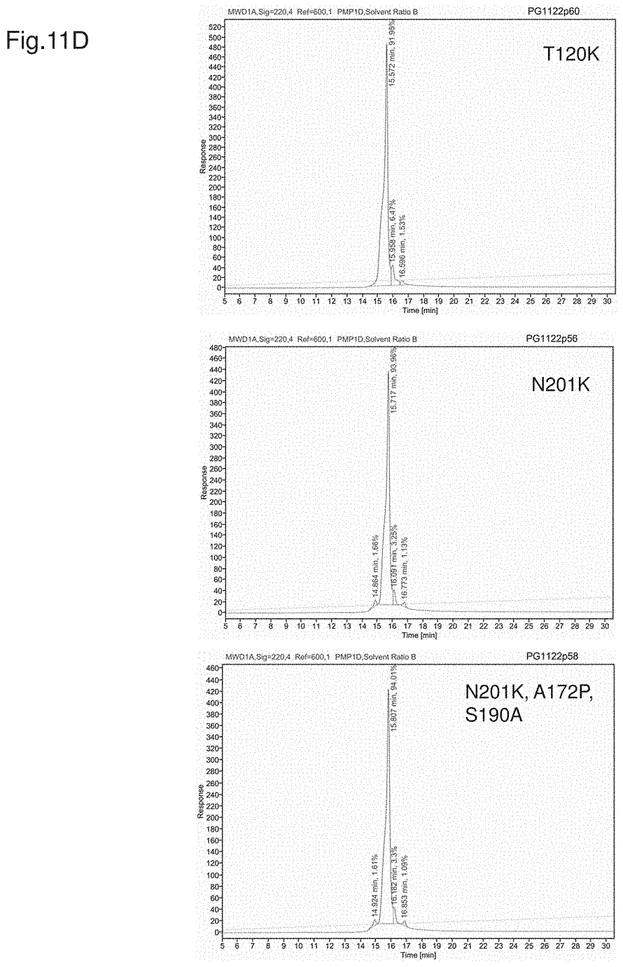

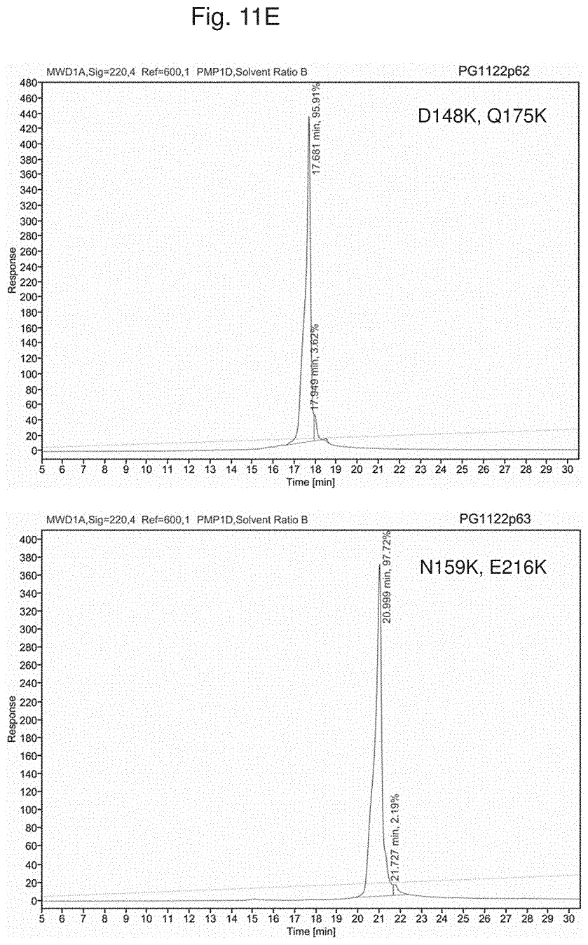

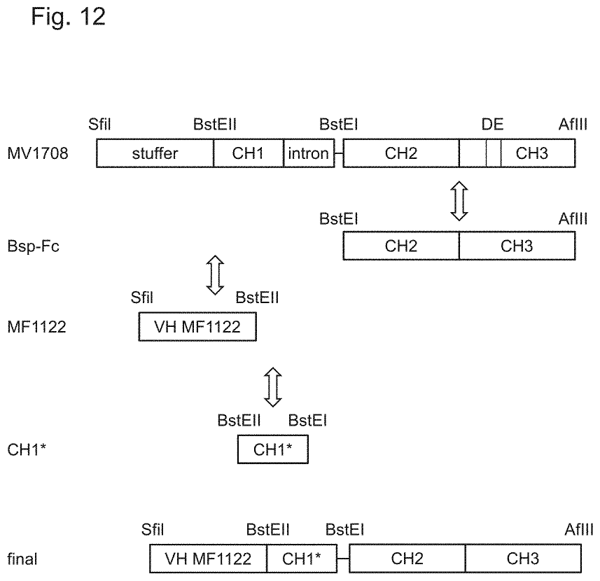

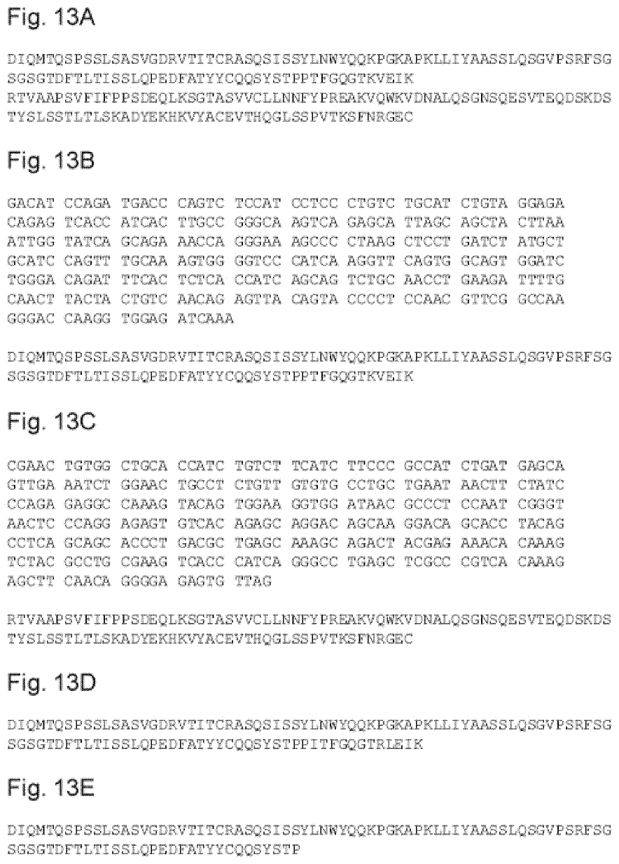

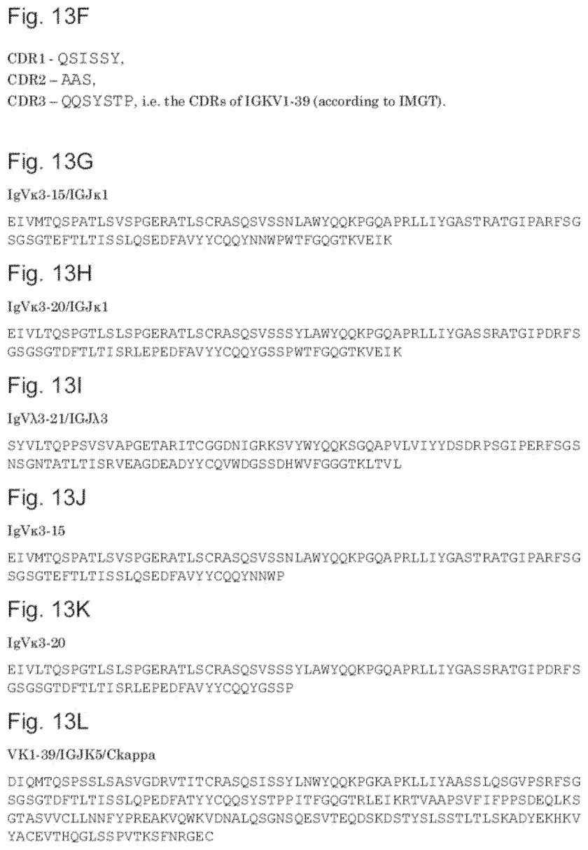

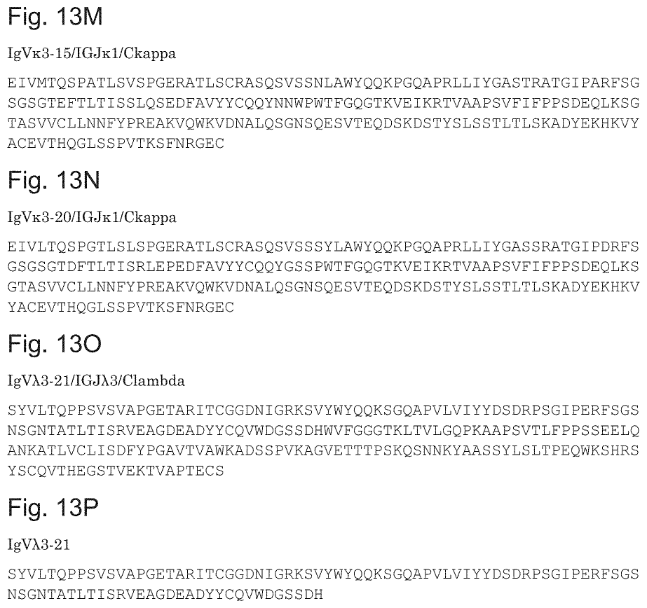

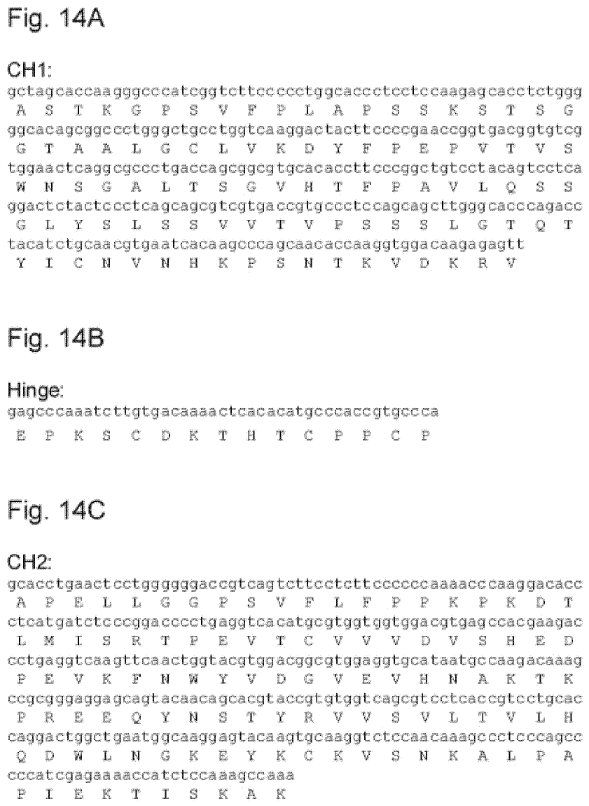

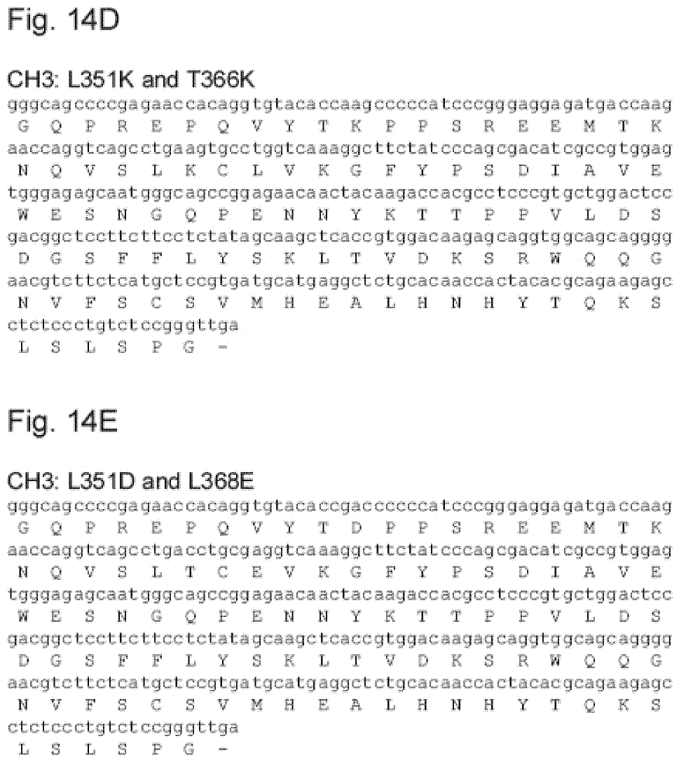

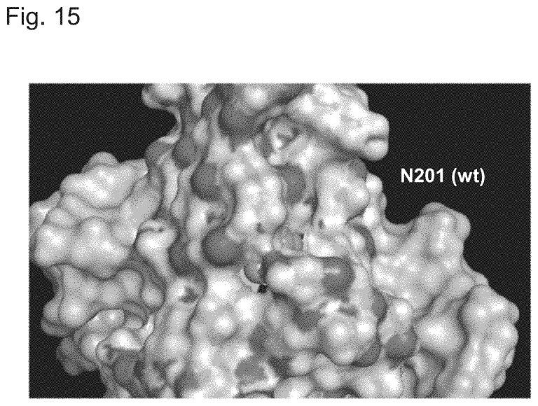

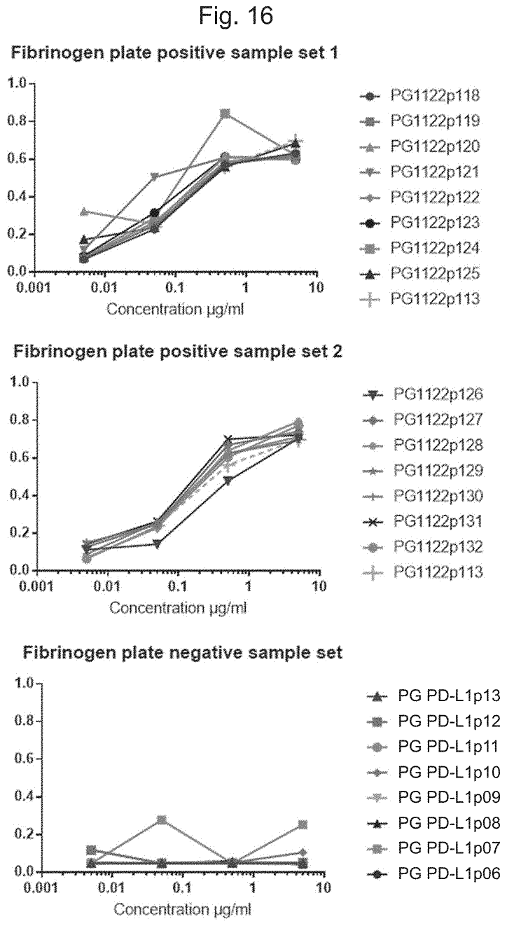

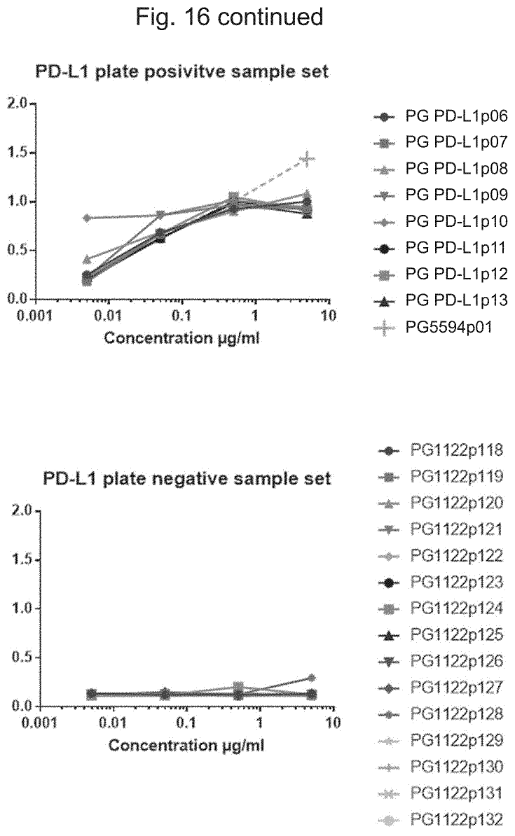

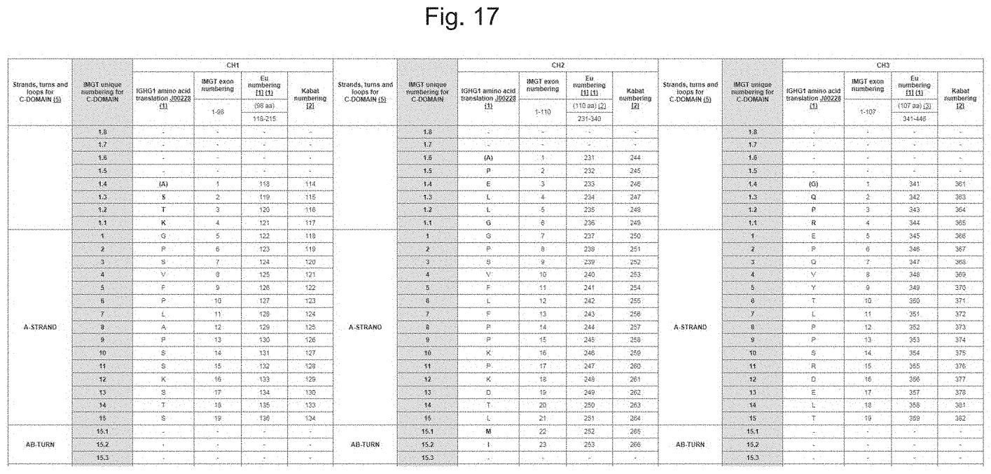

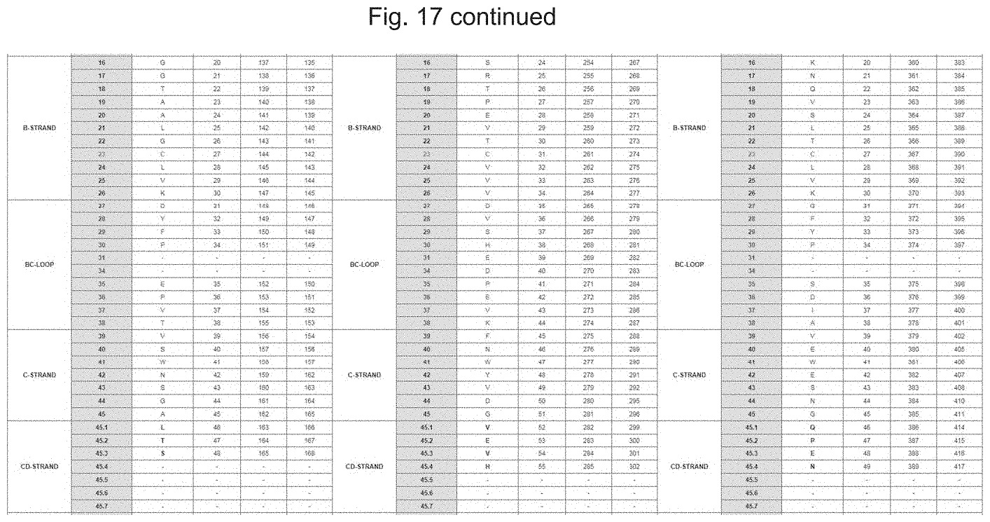

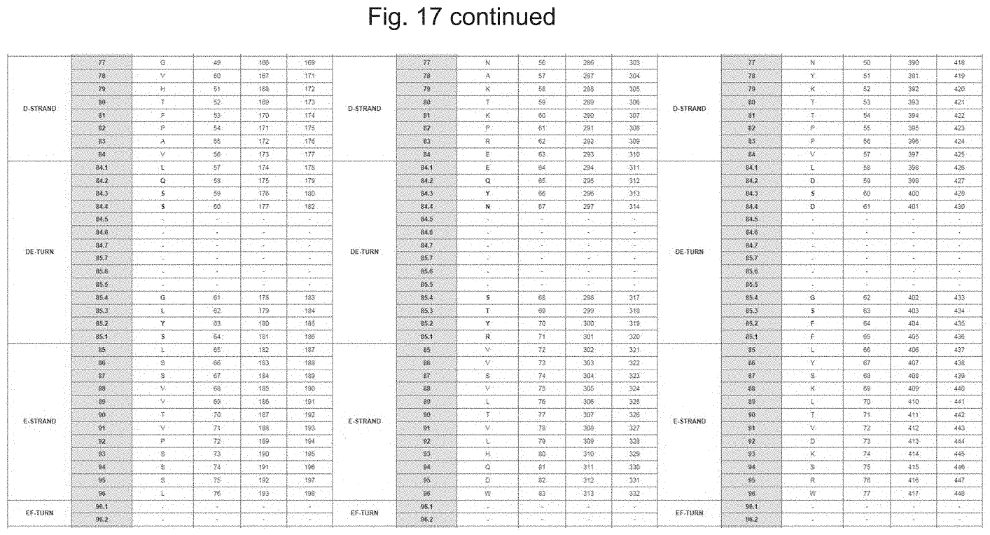

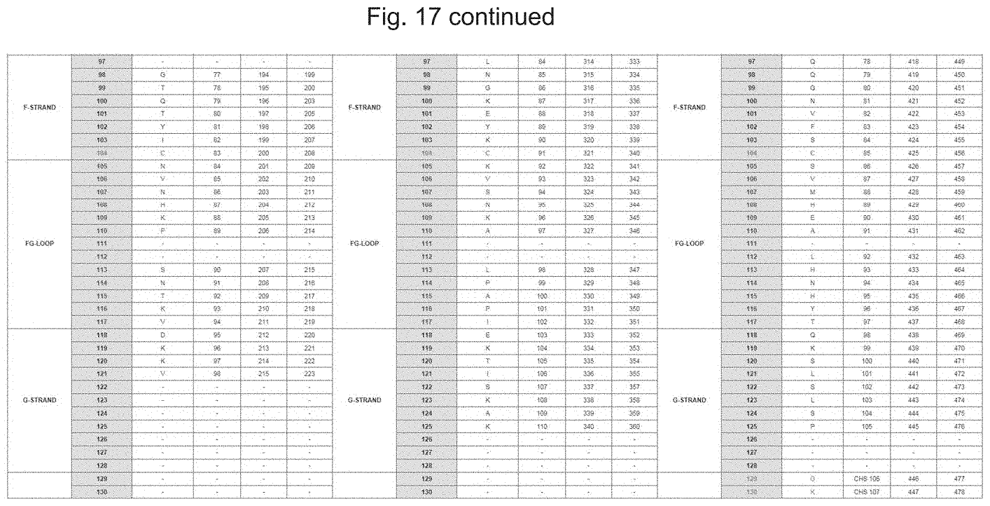

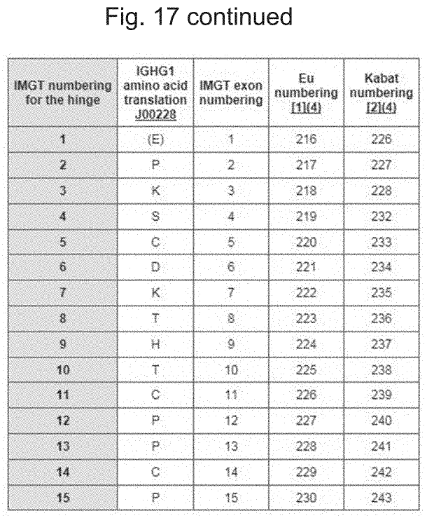

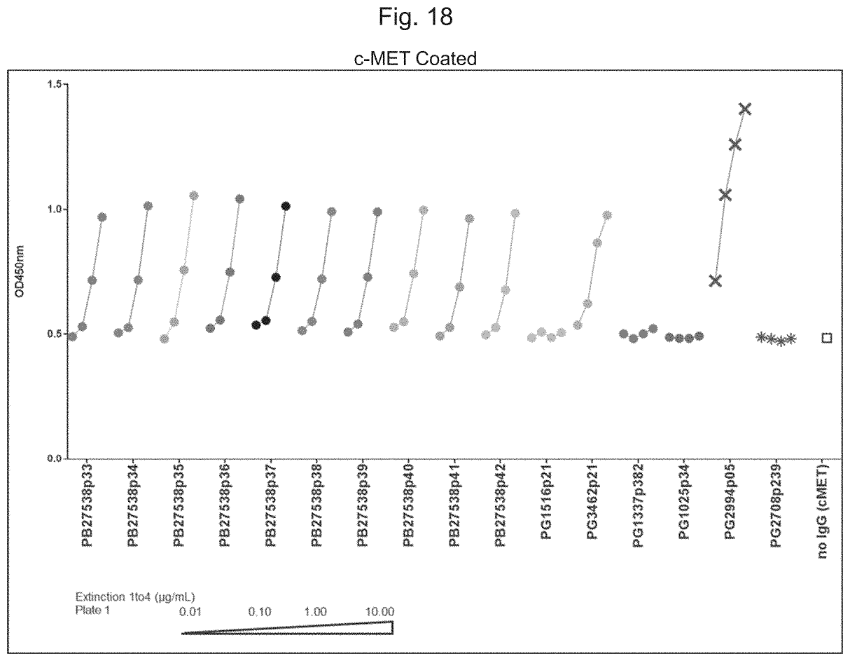

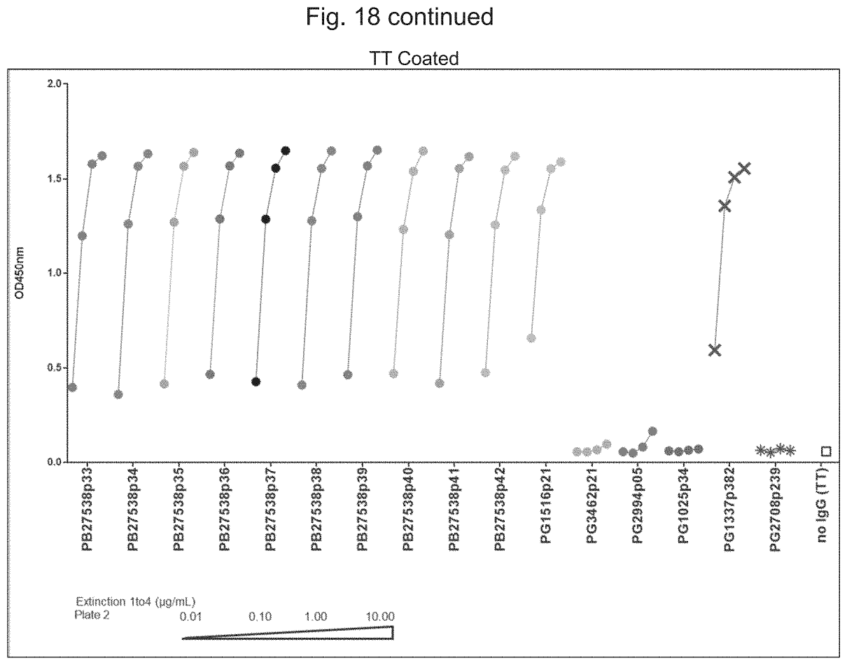

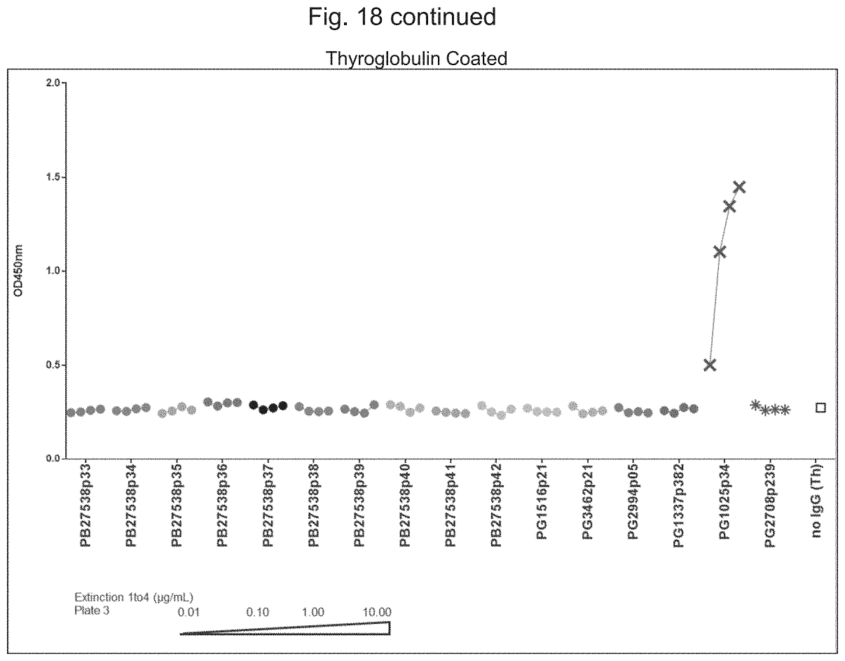

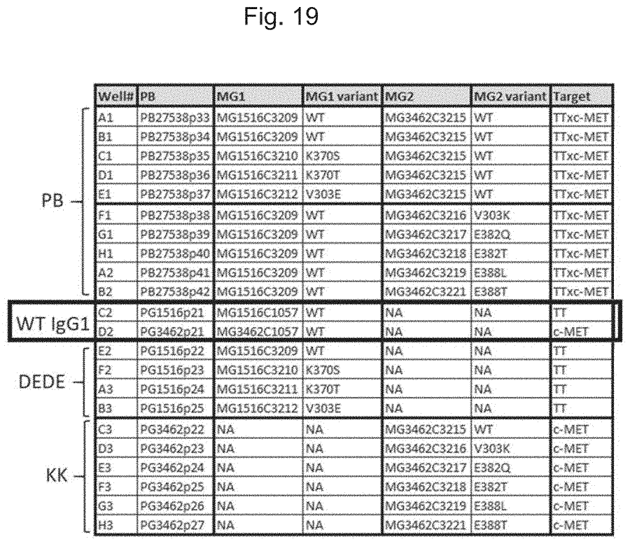

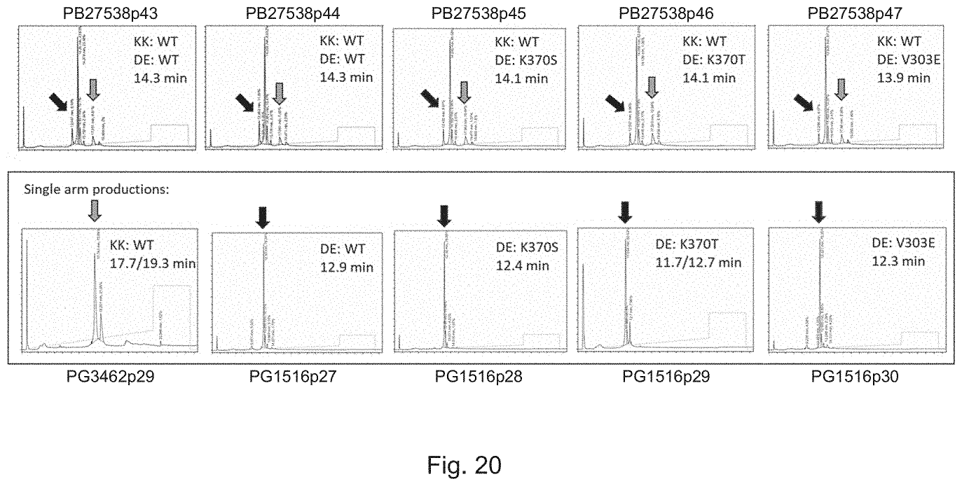

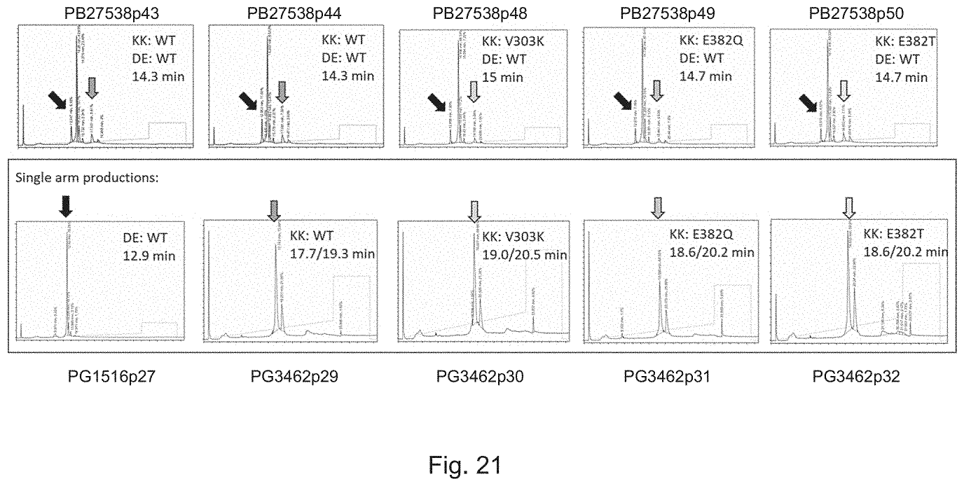

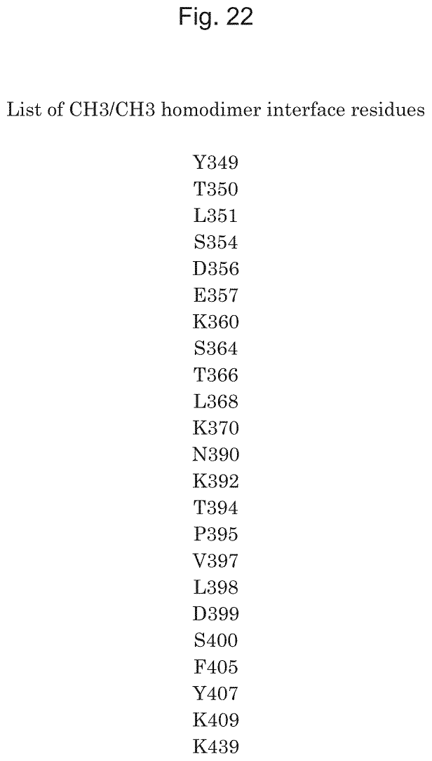

The retention times of the various antibody species as described in Table 13 are displayed in the FIGS. 5-10. As shown in FIG. 5 the CIEX retention time of wild type DE/DE honmodimers and KK halfbodies are relatively close. In contrast, use of the variant CH1 separation domains in conjunction with the DE and KK CH3 heterodimerization domains for these heavy chains alters the CIEX retention time of the heavy chains increasing the difference in retention times for the homodimer, bispecific heterodimer and half bodies. In FIG. 6 the CH1 region of the DE heavy chain with variations T197D and K213Q. The CH1 region of the KK heavy chain with variations N159K and a hinge residue E216K. Therefore the CIEX retention times of the homodimer (DEDE) and the halfbody (KK) have greater difference in retention time as shown in FIG. 6. The retention time of the bispecific antibody (DE/KK) is now further separated from the other species, which allows better separation of the different species.

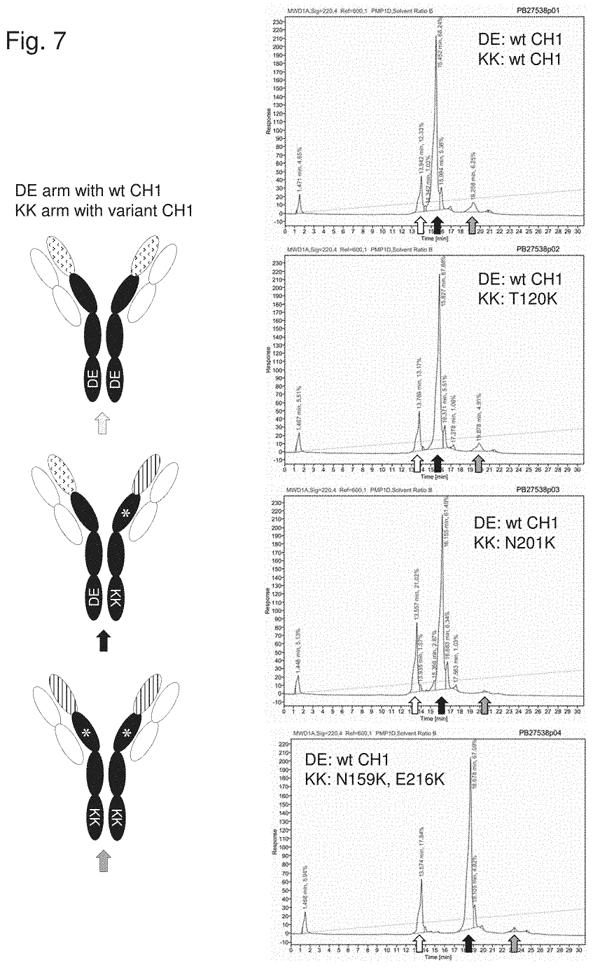

The effect of variations in the CH1 separation domains on the CIEX retention time of bispecific antibodies is shown in FIGS. 7-10.

Table 14
Sequences of CH1, CH2 and CH3 variant separation domains
Part 14A
CH1 WT (SEQ ID NO: 30):
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKX₁V
wherein X₁ = K or R
CH2 WT (SEQ ID NO: 31):
231
APELLGGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA
PIEKTISKAK
CH2 Fc-silent(SEQ ID NO: 32):
231
APELGRGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA
PIEKTISKAK
CH3 WT (SEQ ID NO: 33):
341
GQPREPQVYT LPPSREEMTK NQVSLTCLVK GFYPSDIAVE WESNGQPENN
YKTTPPVLDS DGSFFLYSKL TVDKSRWQQG NVFSCSVMHB ALHNHYTQKS
LSLSPG
Part 14B
CH1 N201K(SEQ ID NO: 34):
ASTKGPSVFPLAPSSKSTSGGTAALGCLVKDYFPEPVTVSWNSGALTSGVHTFPAVLQSSGLYSLSSV
VTVPSSSLGTQTYICKVNHKPSNTKVDKRV
CH1 N201D(SEQ ID NO: 35):
ASTKGPSVFPIAPSSKSTSGGTAALGCLVKDYFPEPVTVSWNSGALTSGVHTFPAVLQSSGLYSLSSV
VTVPSSSLGTQTYICDVNHKPSNTKVDKRV
CH1 A172P SI90A N201K(SEQ ID NO: 36):
ASTKGPSVFPLAPSSKSTSGGTAALGCLVKDYFPKPVTVSWNSGALTSGVHTFPPVLQSSGLYSLSSV
VTVPASSLGTQTYICKVNHKPSNTKVDKRV
CH1 A172P S19CA N201D(SEQ ID NO: 37):
ASTKGPSVFPLAPSSKSTSGGTAALGCLVKDYFPEPVTVSWNSGALTSGVHTFPPVLQSSGLYSLSSV
VTVPASSLGTQTYICDVNHKPSNTKVDKRV
CH1 T120K(SEQ ID NO: 38):
ASKKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKRV
CH1 T120D(SEQ ID NO: 39):
ASDKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKRV
CH1 T197D K213Q (SEQ ID NO: 40):
ASTKGPSVFPLAPSSKSTSGGTAALGCLVKDYFPEPVTVSWNSGALTSGVHTFPAVLQSSGLYSLSSV
VTVPSSSLGTQDYICNVNHKPSNTKVDQRV
CH1 D148K Q175K(SEQ ID NO: 41):
ASTKGPSVFPLAPSSKSTSGGTAALGCLVKKYFPEPVTVSWNSGALTSGVHTFPAVLKSSGLYSLSSV
VTVPSSSLGTQTYICNVNHKPSNTKVDKRV
CH1 N159K(SEQ ID NO: 42):
ASTKGPSVFPLAPSSKSTSGGTAALGCLVKDYFPEPVTVSWKSGALTSGVHTFPAVLQSSGLYSLSSV
VTVPSSSLGTQTYICNVNHKPSNTKVDKRV
CH1 N159D(SEQ ID NO: 43):
ASTKGPSVFPLAPSSKSTSGGTAALGCLVKDYFPEPVTVSWDSGALTSGVHTFPAVLQSSGLYSLSSV
VTVPSSSLGTQTYICNVNHKPSNTKVDKRV
CH1 N159D K213Q(SEQ ID NO: 44):
ASTKGPSVFPIAPSSKSTSGGTAALGCLVKDYFPEPVTVSWDSGALTSGVHTFPAVLQSSGLYSLSSV
VTVPSSSLGTQTYICNVNHKPSNTKVDQRV
CH1 K147E Q175E(SEQ ID NO: 45):
ASTKGPSVFPLAPSSKSTSGGTAALGCLVEDYFPEPVTVSWNSGALTSGVHTFPAVLESSGLYSLSSV
VTVPSSSLGTQTYICNVNHKPSNTKVDKRV
CH1 Y149A VI541 A172P S190A(SEQ ID NO: 46):
ASTKGPSVFPLAPSSKSTSGGTAALGCLVKDAFPEPITVSWNSGALTSGVHTFPPVLQSSGLYSLSSV
VTVPASSLGTQTYICNVNHKPSNTKVDKRV
CH1 K213Q(SEQ ID NO: 47):
ASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDQRV
CH1 N201D K213Q(SEQ ID NO: 48):
ASTKGPSVFPLAPSSKSTSGGTAALGCLVBDYFPEPVTVSWNSGALTSGVHTFPAVLQSSGLYSLSSV
VTVPSSSLGTQTYICDVNHKPSNTKVDQRV
CH1 T120K N2OIK(SEQ ID NO: 49):
ASKKGPSVFPLAPSSKSTSGGTAALGCLVKDYFPEPVTVSWNSGALTSGVHTFPAVLQSSGLYSLSSV
VTVPSSSLGTQTYICKVNHKPSNTKVDKRV
CH1 N201K N159K(SEQ ID NO: 50):
ASTKGPSVFPLAPSSKSTSGGTAALGCLVKDYFPEPVTVSWKSGALTSGVHTFPAVLQSSGLYSLSSV
VTVPSSSLGTQTYICKVNHKPSNTKVDKRV
CH1 T120K N159K(SEQ ID NO: 51):
ASKKGPSVFPLAPSSKSTSGCTAALGCLVKDYFPEPVTVSWKSGALTSGVHTFPAVLQSSGLYSLSSV
VTVPSSSLGTQTYICNVNHKPSNTKVDKRV
CH1 T120K N201K N159K(SEQ ID NO: 52):
ASKKGPSVFPLAPSSKSTSGGTAALGCLVKDYFPEPVTVSWKSGALTSGVHTFPAVLQSSGLYSLSSV
VTVPSSSLGTQTYICKVNHKPSNTKVDKRV
CH1 N201D N159D(SEQ ID NO: 53):
ASTKGPSVFPLAPSSKSTSGGTAALGCLVKDYFPEPVTVSWDSGALTSGVHTFPAVLQSSGLYSLSSV
VTVPSSSLGTQTYICDVNHKPSNTKVDKRV
CH1 N201D K213Q N159D{SEQ ID NO: 54):
ASTKGPSVFPLAPSSKSTSGGTAALGCLVKDYFPEPVTVSWDSGALTSGVHTFPAVLQSSGLYSLSSV
VTVPSSSLGTQTYICDVNHKPSNTKVDQRV
CH2 V303E(SEQ ID NO: 55):
231
APELLGGPSV FLFPPKPKDT LMISRTPEVT CVVVDVSHED PEVKFNWYVD
GVEVHNAKTK PREEQYNSTY RVESVLTVLH QDWLNGKEYK CKVSNKALPA
PIEKTISKAK
CH2 V303K{SEQ ID NO: 56):
231
APELLGGPSV FLFPPKPKDT LMISRTPEVT CVVVDVSHED PEVKFNWYVD
GVEVHNAKTK PREEQYNSTY RVKSVLTVLH QDWLNGKEYK CKVSNKALPA
PIEKTISKAK
CH2 V303E Fc-silent(SEQ ID NO: 57):
231
APELGRGPSV FLFPPKPKDT LMISRTPEVT CVVVDVSHED PEVKFNWYVD
GVEVHNAKTK PREEQYNSTY RVESVLTVLH QDWLNGKEYK CKVSNKALPA
PIEKTISKAK
CH2 V303K Fc-silent (SEQ ID NO: 58):
231
APELGRGPSV FLFPPKPKDT LMISRTPEVT CVVVDVSHED PEVKFNWYVD
GVEVHNAKTK PREEQYNSTY RVKSVLTVLH QDWLNGKEYK CKVSNKALPA
PIEKTISKAK
CH3 K370S(SEQ ID NO: 59) :
341
GQPREPQVYTLPPSREEMTK NQVSLTCLVSGFYPSDIAVE WESNGQPENNYKTTPPVLDS
DGSFFLYSKLTVDKSRWQQG NVFSCSVMHEALHNHYTQKS LSLSPG
CH3 K370T (SEQ ID NO: 60):
341
GQPREPQVYTLPPSREEMTK NQVSLTCLVTGFYPSDIAVE WESNGQPENNYKTTPPVLDS
DGSFFLYSKLTVDKSRWQQG NVFSCSVMHEALHNHYTQKS LSLSPG
CH3 E382Q (SEQ ID NO: 61):
341
GQPREPQVYTLPPSREEMTK NQVSLTCLVKGFYPSDIAVE WQSNGQPENNYKTTPPVLDS
DGSFFLYSKLTVDKSRWQQG NVFSCSVMHEALHNHYTQKS LSLSPG
CH3 E382T(SEQ ID NO: 62):
341
GQPREPQVYTLPPSREEMTK NQVSLTCLVKGFYPSDIAVE WTSNGQPENNYKTTPPVLDS
DGSFFLYSKLTVDKSRWQQG NVFSCSVMHEALHNHYTQKS LSLSPG
CH3 E383L(SEQ ID NO: 63):
341
GQPREPQVYTLPPSREEMTK NQVSLTCLVKGFYPSDIAVE WESNGQPLNNYKTTPPVLDS
DGSFFLYSKLTVDKSRWQQG NVFSCSVMHEALHNHYTQKS LSLSPG
CH3 E388M(SEQ ID NO: 64):
341
GQPREPQVYTLPPSREEMTK NQVSLTCLVKGFYPSDIAVE WESNGQPMNNYKTTPPVLDS
DGSFFLYSKLTVDKSRWQQG NVFSCSVMHEALHNHYTQKS LSLSPG
CH3 E388T(SEQ ID NO: 65):
341
GQPREPQVYTLPPSREEMTK NQVSLTCLVKGFYPSDIAVE WESNGQPTNNYKTTPPVLDS
DGSFFLYSKLTVDKSRWQQG NVFSCSVMHEALHNHYTQKS LSLSPG
CH3 L351K; T366K; H382Q{SEQ ID NO: 66):
341
GQPREPQVYTCPPSREEMTK NQVSLKCLVKGFYPSDIAVE WQSNGQPENNYKTTPPVLDS
DGSFFLYSKLTVDKSRWQQG NVFSCSVMHEALHNHYTQKS LSLSPG
CH3 L351K; T366K; E382T(SEQ ID NO: 67):
341
GQPREPQVYTKPPSREEMTK NQVSLKCLVKGFYPSDIAVE WTSNGQPENNYKTTPPVLDS
DGSFFLYSKLTVDKSRWQQG NVFSCSVMHEALHNHYTQKS LSLSPG
CH3 L351K; T366K; E388L(SEQ ID NO: 68):
341
GQPREPQVYTKPPSREEMTK NQVSLKCLVKGFYPSDIAVE WESNGQPLNNYKTTPPVLDS
DGSFFLYSKLTVDKSRWQQG NVFSCSVMHEALHNHYTQKS LSLSPG
CH3 L351K; T366K; E388M(SEQ ID NO: 69):
341
GQPREPQVYTKPPSREEMTK NQVSLKCLVKGFYPSDIAVE WESNGQPMNNYKTTPPVLDS
DGSFFLYSKLTVDKSRWQQG NVFSCSVMHEALHNHYTQKS LSLSPG
CH3 L351K/T366K; E388T(SEQ ID NO: 70):
341
GQPREPQVYTKPPSREEMTK NQVSLKCLVKGFYPSDIAVE WESNGQPTNNYKTTPPVLDS
DGSFFLYSKLTVDKSRWQQG NVFSCSVMHEALHNHYTQKS LSLSPG
CH3 L351D; L308E; K370S(SEQ ID NO: 71):
341
GQPREPQVYTDPPSRKEMTK NQVSLTCEVSGFYPSDIAVE WESNGQPENNYKTTPPVLDS
DGSFFLYSKLTVDKSRWQQG NVFSCSVMHEALHNHYTQKS LSLSPG
341
GQPREPQVYTDPPSREEMTK NQVSLTCEVTGFYPSDIAVE WESNWPENNYKTIPPVLDS
DGSFFLYSKLIVDKSRWQQG NVFSCSVMHEALHNHYTQKS LSLSPG

### Example 6 Further Analysis of CH1 Variants of Examples 1-5 and New CH1 Variants

Antibodies are produced as indicated in example 1 with the proviso that the antibodies in this example are monospecific bivalent antibodies having two identical heavy chains and a two identical light, chains. As the antibodies are not bispecific antibodies they have a wild type CH3 domain.

ELISA for evaluating the binding to fibrinogen coated plates of various CH1 variants. The antibodies are IgG1 antibodies with the indicated CH1 variants. All antibodies are bivalent monospecific antibodies with variable domains with the VH of MF1122 and the common light chain of FIG. 13A (indicated as PG1122). As a negative control the same antibodies but now with a PD-1L1 antibody VH (indicated as PG PD-1) were tested on the same fibrinogen coated plates (see FIG. 16: respectively Fibrinogen plate positive sample set 1, Fibrinogen plate positive sample set 2 and Fibrinogen plate negative sample set. The same antibodies were evaluated for binding to PD-L1 coated plates (see FIG. 16 PD-L1 plate positive sample set and PD-L1 plate negative sample set).

Fibrinogen ELISA plates were coated with human fibrinogen at 10 μg/ml (Sigma Aldrich; cat. no. F4753). Antibodies were incubated in a ten-fold concentration dilution range starting at 5 μg/ml with an end-concentration of 0.005 μg/ml.

PDI-L1 ELISA plates were coated with human PD-L1-Fc at 2.5 μg/ml (R&D systems; cat. no. 156-B7). Antibodies were incubated in a ten-fold concentration dilution range starting at 5 μg/ml with an end-concentration of 0.005 g/ml. Antibodies that bind were detected with 1:1000 diluted HRP-conjugated Protein L-based secondary antibody which binds the kappa light chain (Pierce cat. no. 32420).

The PD-L1 binding antibodies were taken along as negative controls in the fibrinogen ELISA and the fibrinogen binding antibodies were taken along as negative controls in the PD-L1 ELISA. The negative controls that had the opposite binding specificity but the same CH2, CH3 sequence and CH1 variant sequences as the test antibodies. The amino acid sequence of the MF1122 VH variable region is indicated in table 4. The sequence of the respective CH1 variants is indicated in table 14. Accordingly, these data demonstrate that the separation residues do not impact binding the target antigen of the designated heavy chain variable region.

The conclusion of the ELISA assays is that all the antibodies tested bind to the target specified by the variable domain sequence and importantly not to the non-specific target (FIG. 16). In other words the fibrinogen specific antibodies bind to fibrinogen in the fibrinogen ELISA and not PD-L1 in the PD-L1 ELISA and; the PD-L specific antibodies bind to PD-L1 in the PD-L1 ELSA and not to fibrinogen in the fibrinogen ELSA.

TABLE 15
Antibodies with CH1 variants that introduce charge
differences compared to WT IgG1 CH1.

| CH1 variant | Introduced charge |
|---|---|
| (EU numbering) | compared to wt CH1 |
| N201K | +1 |
| T120K | +1 |
| N159K | +1 |
| T120K, N201K | +2 |
| N201K, N159K | +2 |
| T120K, N159K | +2 |
| T120K, N201K, N159K | +3 |
| N201D | −1 |
| K213Q | −1 |
| N159D | −1 |
| N201D, K213Q | −2 |
| N201D, N159D | −2 |
| K213Q, N159D | −2 |
| N201D, K213Q, N159D | −3 |

The variant indicated were analysed in a WT IgG1 IgG1 background. Also indicated is whether the CH1 variant is associated with a heavy chain having either a DE or KK arm. The heavy chain variable region (VH) of the heavy chain has the sequence of MF1122 (see table 4). CH1 variations that increase the charge (first 7 entries in the figure) have been studied with PD-L1 heavy chain variable region (RT˜11 and FAB Tm˜76° C.).

The various CH1 variant were combined in an otherwise wtIgG1 to produce IgG1 antibodies with the indicated variable domain and CH1 variant. The antibodies are monospecific bivalent antibodies with identical heavy and light chains. Each antibody has two identical CH1 domains and two identical variable domains. The products are analysed with CIEX as described in example 2. The respective CIEX retention times (RT) and relative difference with the retention time of an IgG1 antibody with the same variable domain and a wtCH1 (WT) are indicated in table 16. A larger difference makes it easier to separate the fractions with the bispecific antibody form the homodimers and halfbodies.

TABLE 16
CIEX retention times of bivalent monospecific
antibodies with CH1 separation domains.
Retention time (RT), relative differences (ΔRT)

| | Heavy chain | | | ΔRT |
|---|---|---|---|---|
| CH1 variant | variable region | | | (variant CH1- |
| (EU numbering) | MF | PG | RT | WT CH1) |
| WT | PD-L1 | PG PD-L1p14 | 9.4 | 0 |
| N159K | PD-L1 | PG PD-L1p17 | 12.1 | 2.7 |
| T120K | PD-L1 | PG PD-L1p15 | 12.3 | 2.9 |
| N201K | PD-L1 | PG PD-L1p16 | 12.8 | 3.4 |
| T120K, N159K | PD-L1 | PG PD-L1p20 | 15.1 | 5.7 |
| T120K, N201K | PD-L1 | PG PD-L1p18 | 15.9 | 6.5 |
| N201K, N159K | PD-L1 | PG PD-L1p19 | 17.3 | 7.9 |
| T120K, N201K, | PD-L1 | PG PD-L1p21 | 20.3 | 10.9 |
| N159K | | | | |
| N201D, K213Q, | 1122 | PG1122p147 | 10.2 | −1.7 |
| N159D | | | | |
| K213Q, N159D | 1122 | PG1122p146 | 10.7 | −1.2 |
| N201D, K213Q | 1122 | PG1122p134 | 10.9 | −1 |
| K213Q | 1122 | PG1122p135 | 11.1 | −0.8 |
| N201D, N159D | 1122 | PG1122p145 | 11.4 | −0.5 |
| N159D | 1122 | PG1122p144 | 11.5 | −0.4 |
| WT | 1122 | PG1122p133 | 11.9 | 0 |
| N201D | 1122 | PG1122p136 | 11.9 | 0 |
| N159K | 1122 | PG1122p139 | 13.9 | 2 |
| T120K | 1122 | PG1122p137 | 14.2 | 2.3 |
| N201K | 1122 | PG1122p138 | 14.4 | 2.5 |
| T120K, N159K | 1122 | PG1122p142 | 16.3 | 4.4 |
| T120K, N201K | 1122 | PG1122p140 | 17 | 5.1 |
| N201K, N159K | 1122 | PG1122p141 | 18.3 | 6.4 |
| T120K, N159K, | 1122 | PG1122p143 | 21 | 9.1 |
| N201K | | | | |

Conclusion is that the amino acid variations increase the ΔRT (defined as difference between RT of IgG variant and IgG WT) as expected. Some variants exhibit a larger ΔRT than others. Both tested VH sequences (VH1122 and PD-L1) are affected by the variations to a similar extent.

### Stability Analysis of Antibodies Incorporating the Separation Residues

Example 3 describes an assay for stability with Uncle. The data indicated below are obtained with the Uncle equipment using the method described in example 3.

The monospecific bivalent antibodies indicate in table 16 were tested for various stability parameters. The results are indicated in table 17.

TABLE 17a
PD-L1 VH

| CH1 | | Tm1 | Tm2 | Tm3 | Tagg |
|---|---|---|---|---|---|
| variant | Sample | (° C.) | (° C.) | (° C.) | 266 (° C.) |
| N201K | 0.05 mg/ml | 70.3 | 74.2 | 78.7 | 80.2 |
| T120K | 0.05 mg/ml | 70.6 | 74.5 | 79.0 | 80.0 |
| N159K | 0.05 mg/ml | 66.6 | 70.6 | 78.0 | 79.7 |
| WT | 0.05 mg/ml | 70.2 | 78.0 | | 79.3 |
| N201K, | 0.05 mg/ml | 71.2 | 78.0 | | 79.0 |
| N159K | | | | | |
| T120K, | 0.05 mg/ml | 71.0 | 76.7 | | 78.6 |
| N201K | | | | | |
| T120K, | 0.05 mg/ml | 70.7 | | | 77.7 |
| N159K | | | | | |
| T120K, | 0.05 mg/ml | 71.2 | | | 76.6 |
| N159K, | | | | | |
| N201K | | | | | |

TABLE 17b
VH of MF1122

| CH1 | | Tm1 | Tm2 | Tm3 | Tagg |
|---|---|---|---|---|---|
| variant | Sample | (° C.) | (° C.) | (° C.) | 266 (° C.) |
| WT | 0.05 mg/ml | 70.5 | 80.0 | | 81.7 |
| K213Q | 0.05 mg/ml | 70.5 | 79.7 | | 81.0 |
| T120K | 0.05 mg/ml | 71.1 | 79.0 | | 79.6 |
| N201K | 0.05 mg/ml | 70.5 | 79.0 | | 79.6 |
| N159D | 0.05 mg/ml | 70.6 | 78.2 | | 79.6 |
| N201K, | 0.05 mg/ml | 69.7 | 78.6 | | 79.0 |
| N159K | | | | | |
| N201D | 0.05 mg/ml | 70.0 | 78.1 | | 78.9 |
| N201D, | 0.05 mg/ml | 70.5 | 77.5 | | 78.7 |
| K213Q | | | | | |
| N159K | 0.05 mg/ml | 70.5 | 78.5 | | 78.7 |
| K213Q, | 0.05 mg/ml | 70.2 | 77.6 | | 77.9 |
| N159D | | | | | |
| T120K, | 0.05 mg/ml | 70.1 | 75.0 | | 77.6 |
| N201K | | | | | |
| N201D, | 0.05 mg/ml | 71.0 | | | 77.0 |
| N159D | | | | | |
| T120K, | 0.05 mg/ml | 71.0 | | | 76.3 |
| N159K, | | | | | |
| N201K | | | | | |
| T120K, | 0.05 mg/ml | 70.5 | 75.0 | | 75.9 |
| N159K | | | | | |
| N201D, | 0.05 mg/ml | 70.0 | | | 75.7 |
| K213Q, | | | | | |
| N159D | | | | | |

In all cases combination of several variations decreases TAGG, however the decrease is well within tolerance levels. Overall thermal stability is affected for both VH to similar extent, and evidenced to be independent on the specific VH sequence in the associated variable domain. Modification of N159 to with the anti-PD-L1 containing variable domain is in this analysis associated with an early melting event at ˜66 degree. This is likely measuring issue as the value for this variant with the MF122 containing variable domain does not show this same difference with WT. This trend is also seen in the various combinations with N159K which typically do not show the same different with WT.

TABLE 18
Summary for all variants that increase the charge or the pI of an antibody
with the variant when compared to an antibody with a WT CH1 sequence.

| | VH PD-L1 | | | | | | VH1122 | | | | |
|---|---|---|---|---|---|---|---|---|---|---|---|
| | | | Thermal Stability | | | | | | Thermal Stability | | |
| | CIEX | | | | | Tagg | CIEX | | | | Tagg |
| CH1 | | ΔRT | Tm1 | Tm2 | Tm3 | 266 | | ΔRT | Tm1 | Tm2 | 266 |
| variant | RT | (variant − WT) | (° C.) | (° C.) | (° C.) | (° C.) | RT | (variant − WT) | (° C.) | (° C.) | (° C.) |
| WT | 9.4 | 0 | 70.2 | 78.0 | | 79.3 | 11.9 | 0 | 70.5 | 80.0 | 81.7 |
| N159K | 12.1 | 2.7 | 66.6 | 70.6 | 78.0 | 79.7 | 13.9 | 2 | 70.5 | 78.5 | 78.7 |
| T120K | 12.3 | 2.9 | 70.6 | 74.5 | 79.0 | 80.0 | 14.2 | 2.3 | 71.1 | 79.0 | 79.6 |
| N201K | 12.8 | 3.4 | 70.3 | 74.2 | 78.7 | 80.2 | 14.4 | 2.5 | 70.5 | 79.0 | 79.6 |
| T120K, | 15.1 | 5.7 | 70.7 | | | 77.7 | 16.3 | 4.4 | 70.5 | 75.0 | 75.9 |
| N159K | | | | | | | | | | | |
| T120K, | 15.9 | 6.5 | 71.0 | 76.7 | | 78.6 | 17.0 | 5.1 | 70.1 | 75.0 | 77.6 |
| N201K | | | | | | | | | | | |
| N201K, | 17.3 | 7.9 | 71.2 | 78.0 | | 79.0 | 18.3 | 6.4 | 69.7 | 78.6 | 79.0 |
| N159K | | | | | | | | | | | |
| T120K, | 20.3 | 10.9 | 71.2 | | | 76.6 | 21.0 | 9.1 | 71.0 | | 76.3 |
| N159K, | | | | | | | | | | | |
| N201K | | | | | | | | | | | |

When compared to the other CH1 variants listed in table 18, it appears that of the single amino acid variants N201K causes strongest shift on CIEX (2.5-3.4 minutes) while maintaining high thermal stability (TAGG is reduced by 0-2° C.). This is the case for both tested VH sequences regardless of their antigen binding specificity and the germline V regions they are derived from. The other listed single amino acid variants also exhibit good stability and exhibit useful shift in CIEX retention time.

TABLE 19
Summary for all variants that alter the charge or the pI of an antibody
with the variant when compared to an antibody with a WT CH1 sequence.

| | | | | Thermal Stability | | | |
|---|---|---|---|---|---|---|---|
| | | CIEX | | | | Tagg | |
| | CH1 | | ΔRT | Tm1 | Tm2 | 266 | ΔTAGG |
| pI | variant | RT | (variant − WT) | (° C.) | (° C.) | (° C.) | (variant − WT) |
| lower | N201D, | 10.2 | −1.7 | 70.0 | | 75.7 | −6.0 |
| | K213Q, | | | | | | |
| | N159D | | | | | | |
| lower | K213Q, | 10.7 | −1.2 | 70.2 | 77.6 | 77.9 | −3.9 |
| | N159D | | | | | | |
| lower | N201D, | 10.9 | −1.0 | 70.5 | 77.5 | 78.7 | −3.0 |
| | K213Q | | | | | | |
| lower | K213Q | 11.1 | −0.8 | 70.5 | 79.7 | 81.0 | −0.7 |
| lower | N201D, | 11.4 | −0.5 | 71.0 | | 77.0 | −4.8 |
| | N159D | | | | | | |
| lower | N159D | 11.5 | −0.4 | 70.6 | 78.2 | 79.6 | −2.1 |
| lower | N201D | 11.9 | 0.0 | 70.0 | 78.1 | 78.9 | −2.8 |
| | WT | 11.9 | 0.0 | 70.5 | 80.0 | 81.7 | 0.0 |
| higher | N159K | 13.9 | 2.0 | 70.5 | 78.5 | 78.7 | −3.0 |
| higher | T120K | 14.2 | 2.3 | 71.1 | 79.0 | 79.6 | −2.1 |
| higher | N201K | 14.4 | 2.5 | 70.5 | 79.0 | 79.6 | −2.1 |
| higher | T120K, | 16.3 | 4.4 | 70.5 | 75.0 | 75.9 | −5.8 |
| | N159K | | | | | | |
| higher | T120K, | 17.0 | 5.1 | 70.1 | 75.0 | 77.6 | −4.1 |
| | N201K | | | | | | |
| higher | N201K, | 18.3 | 6.4 | 69.7 | 78.6 | 79.0 | −2.8 |
| | N159K | | | | | | |
| higher | T120K, | 21.0 | 9.1 | 71.0 | | 76.3 | −5.4 |
| | N159K, | | | | | | |
| | N201K | | | | | | |

The variants tested all have similar Tm1 values whereas Tm2 is within a suitable range. A single variation K213Q causes a strong shift on CIEX (−0.8 minutes) while maintaining good thermal stability (TAGG is reduced by 0.7° C.). Double variant N201K+N159K provides a pronounced effect on CIEX retention while having a limited effect on thermal stability. The tested triple variant in this case had the largest CIEX retention time shift.

### Example 7a: Identifying Non-Surface Residues for Separation Design

From structural information of an IgG1 12 region with another CH2 region surface, non-surface exposed and buried amino acid residue positions within the CH2 regions were identified by use of the program GETAREA 1.0 using default parameters. Negi et al., “Solvent Accessible Surface Areas. Atomic Solvation Energies, and Their Gradients for Macromolecules”, Last modified on Wed 17th April, 3:00 PM, 2015. A model of the CH2 region with the sequence of table 20 was submitted to the Swiss-model website (Arnold K, Bordoli L. Kopp J, Schwede T. The SWISS-MODEL workspace; a web-based environment for protein structure homology modelling. Bioinformatics, 2006 Jan. 15; 22(2):195-201). The same was done for the IgG1 CH3 region.

High quality CH2 and CH3 homology models were obtained essentially as explained above Swiss-Model version 1.3.0 was used from the Swiss-Model web server. Several appropriate crystal structures exist (that are high quality structures and have alignments with >95% sequence identity to the CH2 domain used as the ‘original’ or template sequence in many of the embodiments herein). Numerous other CH2 regions in the PDB could provide high quality starting structures (with >95% sequence identity to the CH2 region used here and high quality structures). Many are readily identified using commonly used homology modeling tools, as in Example 1. A structural model of the CH2 domain was obtained with 98.2% sequence identity over the full length starting from the PDB template 5vu0 of the CH2 query sequence (note that the mismatches occur in engineered regions and in terminal/linker regions, and that the model obtains a Swiss-Model GMQE score of 0.99, version 1.3.0).

This structure was processed with GETAREA 1.0 beta, uploading the pdb file generated by Swiss-Model. Based on the default parameters of GETAREA 1.0 beta, amino acids that are greater than 50% surface exposed are referred to as “Out” or surface, non-OUT or non-surface exposed residues are between 50% to greater than 20%, and less than 20% accessible are referred by GETAREA 1.0 beta as “In” or as referred to herein as buried amino acids

### CH3

Similarly, the ‘original’ or any engineered CH3 domain embodied herein can be modelled as a homo-dimer (two CH3 chains interacting) or as a monomer using homology modelling. Several appropriate crystal structures exist (that are high quality structures and have alignments with >92% sequence identity to the DE- or KK-CH3 domain used as the ‘original’ sequence in many of the embodiments herein). Numerous other CH3 regions in the PDB could provide high quality starting structures (with >92% sequence identity to the CH3 region used here and high quality structures). Many are readily identified using commonly used homology modelling tools, as in Example 1. For example we produce a structural model of the CH3 domain (DE-CH3) with 93.46 sequence identity over the full length starting from the PDB template 5w38 of the DE-CH3 query sequence (note that the mismatches occur in engineered regions and in linker/domain-terminal regions, and that the model obtains a Swiss-Model GMQE score of 0.99, version 1.3.0). This structure was processed with GETAREA 1.0 beta, uploading the pdb file generated by Swiss-Model. Based on the default parameters of GETAREA 1.0 beta, amino acids that are greater than 50% surface exposed are referred to as “Out” or surface, non-OUT or non-surface exposed residues are between 50% to greater than 20%, and less than 20% accessible are referred by GETAREA 1.0 beta as “In” or as referred to herein as buried amino acids (see tables 20-22).

The CH2 region modelled is a human CH2, modified for silencing at positions 235 and 236 according to EU numbering. The CH3 regions modelled are the human CH3 modified to include a L351D and L368E variation for table 21 and a T366K and L351K variation for table 22 according to EU numbering, thereby modelling the CH3 chains for the CH3 DEKK heterodimerization domain.

TABLE 20
CH2 Fc-silent GetArea Scoring.
Probe radius: 1.400

| Residue | | Total | Apolar | Backbone | Sidechain | Ratio (%) | In/Out |
|---|---|---|---|---|---|---|---|
| PRO | 1 | 193.74 | 162.89 | 55.64 | 138.10 | 100.0 | o |
| ALA | 2 | 103.03 | 77.89 | 29.68 | 73.36 | 100.0 | o |
| PRO | 3 | 132.81 | 115.27 | 28.97 | 103.84 | 98.7 | o |
| GLU | 4 | 145.43 | 55.55 | 19.06 | 126.37 | 89.5 | o |
| LEU | 5 | 135.97 | 121.61 | 21.70 | 114.27 | 78.2 | o |
| GLY | 6 | 52.91 | 45.33 | 52.91 | 0.00 | 60.7 | o |
| ARG | 7 | 243.13 | 121.37 | 42.24 | 200.89 | 100.0 | o |
| GLY | 8 | 24.77 | 11.96 | 24.77 | 0.00 | 28.4 | |
| PRO | 9 | 6.33 | 2.66 | 3.67 | 2.66 | 2.5 | i |
| SER | 10 | 43.55 | 16.19 | 3.51 | 40.03 | 51.7 | o |
| VAL | 11 | 4.68 | 0.00 | 4.68 | 0.00 | 0.0 | i |
| PHE | 12 | 108.72 | 108.72 | 3.15 | 105.58 | 58.6 | o |
| LEU | 13 | 21.40 | 4.52 | 16.89 | 4.52 | 3.1 | i |
| PHE | 14 | 102.35 | 102.35 | 0.02 | 102.34 | 56.8 | o |
| PRO | 15 | 63.34 | 56.68 | 6.66 | 56.68 | 53.9 | o |
| PRO | 16 | 9.46 | 4.14 | 9.43 | 0.04 | 0.0 | i |
| LYS | 17 | 138.82 | 101.00 | 0.64 | 138.18 | 84.0 | o |
| PRO | 18 | 97.41 | 97.41 | 5.82 | 91.58 | 87.1 | o |
| LYS | 19 | 120.28 | 81.58 | 2.93 | 117.34 | 71.3 | o |
| ASP | 20 | 24.23 | 0.45 | 0.00 | 24.23 | 21.4 | |
| THR | 21 | 5.50 | 0.92 | 1.27 | 4.24 | 4.0 | i |
| LEU | 22 | 120.34 | 95.71 | 26.86 | 93.48 | 63.9 | o |
| MET | 23 | 114.40 | 114.40 | 3.68 | 110.71 | 69.9 | o |
| ILE | 24 | 147.67 | 140.44 | 8.78 | 138.90 | 94.3 | o |
| SER | 25 | 108.79 | 61.18 | 38.59 | 70.20 | 90.7 | o |
| ARG | 26 | 99.39 | 33.11 | 8.74 | 90.65 | 46.4 | |
| THR | 27 | 84.29 | 45.25 | 13.94 | 70.35 | 66.2 | o |
| PRO | 28 | 2.03 | 2.03 | 0.67 | 1.35 | 1.3 | i |
| GLU | 29 | 74.03 | 11.89 | 2.36 | 71.67 | 50.8 | o |
| VAL | 30 | 0.00 | 0.00 | 0.00 | 0.00 | 0.0 | i |
| THR | 31 | 31.24 | 20.30 | 0.00 | 31.24 | 29.4 | |
| CYS | 32 | 0.00 | 0.00 | 0.00 | 0.00 | 0.0 | i |
| VAL | 33 | 11.02 | 11.02 | 0.00 | 11.02 | 9.0 | i |
| VAL | 34 | 0.00 | 0.00 | 0.00 | 0.00 | 0.0 | i |
| VAL | 35 | 37.54 | 37.54 | 0.00 | 37.54 | 30.7 | |
| ASP | 36 | 70.47 | 15.73 | 9.38 | 61.09 | 54.1 | o |
| VAL | 37 | 0.19 | 0.19 | 0.12 | 0.07 | 0.1 | i |
| SER | 38 | 44.61 | 40.83 | 9.52 | 35.09 | 45.3 | |
| HIS | 39 | 88.36 | 62.17 | 13.44 | 74.92 | 48.5 | |
| GLU | 40 | 149.40 | 73.31 | 30.26 | 119.14 | 84.4 | o |
| ASP | 41 | 28.81 | 1.73 | 0.89 | 27.92 | 24.7 | |
| PRO | 42 | 25.65 | 25.04 | 0.61 | 25.04 | 23.8 | |
| GLU | 43 | 117.21 | 55.73 | 6.38 | 110.83 | 78.5 | o |
| VAL | 44 | 23.94 | 1.82 | 23.00 | 0.94 | 0.8 | i |
| LYS | 45 | 124.86 | 98.22 | 5.73 | 119.13 | 72.4 | o |
| PHE | 46 | 24.15 | 5.11 | 19.04 | 5.11 | 2.8 | i |
| ASN | 47 | 24.44 | 4.30 | 4.30 | 20.14 | 17.6 | i |
| TRP | 48 | 4.21 | 3.88 | 0.33 | 3.88 | 1.7 | i |
| TYR | 49 | 37.06 | 26.77 | 0.05 | 37.01 | 19.2 | i |
| VAL | 50 | 22.89 | 21.30 | 1.59 | 21.30 | 17.4 | i |
| ASP | 51 | 75.33 | 37.74 | 25.52 | 49.81 | 44.1 | |
| GLY | 52 | 60.12 | 33.43 | 60.12 | 0.00 | 68.9 | o |
| VAL | 53 | 110.66 | 110.66 | 4.89 | 105.77 | 86.5 | o |
| GLU | 54 | 66.84 | 18.07 | 13.03 | 53.80 | 38.1 | |
| VAL | 55 | 40.38 | 36.18 | 7.70 | 32.69 | 26.7 | |
| HIS | 56 | 157.04 | 105.36 | 35.49 | 121.55 | 78.6 | o |
| ASN | 57 | 89.31 | 55.03 | 22.99 | 66.32 | 58.0 | o |
| ALA | 58 | 31.86 | 8.47 | 23.55 | 8.32 | 12.8 | i |
| LYS | 59 | 150.21 | 106.48 | 6.84 | 143.36 | 87.2 | o |
| THR | 60 | 72.64 | 36.55 | 27.94 | 44.70 | 42.1 | |
| LYS | 61 | 106.53 | 65.14 | 6.32 | 100.21 | 60.9 | o |
| PRO | 62 | 121.87 | 119.66 | 16.64 | 105.23 | 100.0 | o |
| ARG | 63 | 91.66 | 20.30 | 14.82 | 76.84 | 39.3 | |
| GLU | 64 | 109.22 | 38.87 | 6.04 | 103.18 | 73.1 | o |
| GLU | 65 | 95.40 | 25.89 | 23.90 | 71.51 | 50.6 | o |
| GLN | 66 | 58.41 | 19.49 | 10.88 | 47.54 | 33.1 | |
| TYR | 67 | 237.61 | 174.37 | 31.97 | 205.64 | 100.0 | o |
| ASN | 68 | 105.09 | 50.26 | 26.26 | 78.83 | 69.0 | o |
| SER | 69 | 69.30 | 40.30 | 3.56 | 65.74 | 84.9 | o |
| THR | 70 | 35.84 | 33.97 | 0.92 | 34.92 | 32.9 | |
| TYR | 71 | 43.46 | 10.57 | 0.00 | 43.46 | 22.5 | |
| ARG | 72 | 74.94 | 20.74 | 1.23 | 73.72 | 37.7 | |
| VAL | 73 | 7.91 | 7.91 | 0.54 | 7.36 | 6.0 | i |
| VAL | 74 | 15.80 | 15.67 | 0.12 | 15.67 | 12.8 | i |
| SER | 75 | 0.00 | 0.00 | 0.00 | 0.00 | 0.0 | i |
| VAL | 76 | 12.37 | 11.48 | 0.88 | 11.48 | 9.4 | i |
| LEU | 77 | 2.50 | 2.50 | 0.12 | 2.38 | 1.6 | i |
| THR | 78 | 57.73 | 39.31 | 7.53 | 50.20 | 47.3 | |
| VAL | 79 | 5.26 | 3.64 | 5.26 | 0.00 | 0.0 | i |
| LEU | 80 | 129.98 | 129.25 | 1.46 | 128.52 | 87.9 | o |
| HIS | 81 | 26.08 | 21.14 | 0.61 | 25.47 | 16.5 | i |
| GLN | 82 | 111.49 | 66.99 | 0.51 | 110.98 | 77.2 | o |
| ASP | 83 | 26.33 | 0.36 | 0.48 | 25.85 | 22.9 | |
| TRP | 84 | 18.21 | 10.40 | 0.05 | 18.16 | 8.1 | i |
| LEU | 85 | 53.50 | 44.95 | 8.55 | 44.95 | 30.7 | |
| ASN | 86 | 103.14 | 23.60 | 33.10 | 70.04 | 61.3 | o |
| GLY | 87 | 32.76 | 15.59 | 32.76 | 0.00 | 37.6 | |
| LYS | 88 | 69.15 | 39.00 | 2.63 | 66.52 | 40.4 | |
| GLU | 89 | 85.88 | 19.30 | 0.49 | 85.39 | 60.5 | o |
| TYR | 90 | 2.54 | 2.02 | 0.00 | 2.54 | 1.3 | i |
| LYS | 91 | 59.65 | 27.60 | 0.00 | 59.65 | 36.3 | |
| CYS | 92 | 0.00 | 0.00 | 0.00 | 0.00 | 0.0 | i |
| LYS | 93 | 42.70 | 12.03 | 0.18 | 42.52 | 25.9 | |
| VAL | 94 | 0.00 | 0.00 | 0.00 | 0.00 | 0.0 | i |
| SER | 95 | 28.95 | 24.24 | 1.86 | 27.09 | 35.0 | |
| ASN | 96 | 14.37 | 6.88 | 13.83 | 0.54 | 0.5 | i |
| LYS | 97 | 167.65 | 121.92 | 31.24 | 136.40 | 82.9 | o |
| ALA | 98 | 47.07 | 36.13 | 22.90 | 24.17 | 37.2 | |
| LEU | 99 | 15.12 | 4.67 | 10.64 | 4.49 | 3.1 | i |
| PRO | 100 | 101.58 | 73.35 | 44.85 | 56.73 | 53.9 | o |
| ALA | 101 | 63.20 | 59.97 | 6.37 | 56.84 | 87.6 | o |
| PRO | 102 | 69.59 | 54.52 | 15.36 | 54.23 | 51.5 | o |
| ILE | 103 | 28.78 | 28.78 | 3.94 | 24.83 | 16.9 | i |
| GLU | 104 | 85.12 | 27.43 | 18.14 | 66.98 | 47.4 | |
| LYS | 105 | 86.57 | 44.26 | 5.34 | 81.24 | 49.4 | |
| THR | 106 | 66.88 | 35.96 | 25.68 | 41.20 | 38.8 | |
| ILE | 107 | 35.27 | 35.27 | 6.06 | 29.21 | 19.8 | i |
| SER | 108 | 38.47 | 2.27 | 15.77 | 22.70 | 29.3 | |
| LYS | 109 | 77.36 | 45.77 | 6.40 | 70.96 | 43.1 | |
| ALA | 110 | 94.75 | 83.95 | 25.21 | 69.54 | 100.0 | o |
| LYS | 111 | 252.60 | 132.84 | 47.34 | 205.25 | 100.0 | o |

CH2 sequence and modeling information. Positions are indicated with an arbitrary number. Residue ALA with number 2 corresponds to EU-number 231, residue PRO with number 3 corresponds to EU-number 232 etc until residue LYS with number 111 which has position number 340 according to EU-numbering (see IMGT table depicted in FIG. 17).

The sequence of the C2 region comprises an Fc-silent variation at positions 235 and 236 (an L235G and an G236R variation). Column In/Out indicates whether the amino acid is considered to be buried (i) or surface-exposed (o). An open space indicates a value for an amino acid that is not surface-exposed but also not buried.

TABLE 21
CH3 muts model DE variant
Probe radius: 1.400

| Residue | | Total | Apolar | Backbone | Sidechain | Ratio (%) | In/Out |
|---|---|---|---|---|---|---|---|
| GLY | 1 | 93.09 | 41.16 | 93.09 | 0.00 | 100.0 | o |
| GLN | 2 | 155.68 | 61.13 | 7.43 | 148.25 | 100.0 | o |
| PRO | 3 | 73.51 | 55.46 | 18.05 | 55.46 | 52.7 | o |
| ARG | 4 | 98.09 | 60.52 | 6.40 | 91.69 | 46.9 | |
| GLU | 5 | 86.26 | 24.01 | 5.26 | 81.00 | 57.4 | o |
| PRO | 6 | 4.08 | 0.00 | 4.08 | 0.00 | 0.0 | i |
| GLN | 7 | 73.96 | 26.14 | 6.11 | 67.85 | 47.2 | |
| VAL | 8 | 5.60 | 0.45 | 5.14 | 0.45 | 0.4 | i |
| TYR | 9 | 111.59 | 87.13 | 4.60 | 106.99 | 55.4 | o |
| THR | 10 | 28.41 | 0.00 | 17.68 | 10.74 | 10.1 | i |
| ASP | 11 | 57.13 | 21.71 | 0.01 | 57.12 | 50.5 | o |
| PRO | 12 | 80.78 | 68.74 | 12.04 | 68.74 | 65.3 | o |
| PRO | 13 | 13.72 | 4.86 | 13.59 | 0.13 | 0.1 | i |
| SER | 14 | 66.96 | 55.58 | 17.00 | 49.96 | 64.5 | o |
| ARG | 15 | 202.77 | 95.36 | 10.47 | 192.31 | 98.4 | o |
| GLU | 16 | 155.84 | 60.11 | 24.88 | 130.97 | 92.8 | o |
| GLU | 17 | 44.56 | 18.97 | 3.47 | 41.09 | 29.1 | |
| MET | 18 | 41.84 | 39.31 | 2.53 | 39.31 | 24.8 | |
| THR | 19 | 130.40 | 93.72 | 34.74 | 95.66 | 90.1 | o |
| LYS | 20 | 127.83 | 81.65 | 6.71 | 121.12 | 73.6 | o |
| ASN | 21 | 132.13 | 39.17 | 14.75 | 117.38 | 100.0 | o |
| GLN | 22 | 71.05 | 17.44 | 4.87 | 66.18 | 46.1 | |
| VAL | 23 | 0.40 | 0.40 | 0.40 | 0.00 | 0.0 | i |
| SER | 24 | 25.15 | 14.87 | 0.85 | 24.30 | 31.4 | |
| LEU | 25 | 0.00 | 0.00 | 0.00 | 0.00 | 0.0 | i |
| THR | 26 | 31.92 | 16.70 | 0.00 | 31.92 | 30.1 | |
| CYS | 27 | 0.00 | 0.00 | 0.00 | 0.00 | 0.0 | i |
| GLU | 28 | 36.91 | 0.13 | 0.00 | 36.91 | 26.1 | |
| VAL | 29 | 0.00 | 0.00 | 0.00 | 0.00 | 0.0 | i |
| LYS | 30 | 81.64 | 38.53 | 0.00 | 81.64 | 49.6 | |
| GLY | 31 | 9.38 | 7.19 | 9.38 | 0.00 | 10.8 | i |
| PHE | 32 | 0.00 | 0.00 | 0.00 | 0.00 | 0.0 | i |
| TYR | 33 | 60.64 | 26.00 | 0.00 | 60.64 | 31.4 | |
| PRO | 34 | 41.03 | 41.03 | 0.00 | 41.03 | 39.0 | |
| SER | 35 | 35.30 | 7.93 | 1.45 | 33.85 | 43.7 | |
| ASP | 36 | 117.22 | 39.01 | 10.75 | 106.47 | 94.2 | o |
| ILE | 37 | 37.55 | 12.12 | 31.56 | 5.99 | 4.1 | i |
| ALA | 38 | 57.19 | 56.62 | 8.29 | 48.90 | 75.3 | o |
| VAL | 39 | 17.59 | 2.52 | 16.21 | 1.38 | 1.1 | i |
| GLU | 40 | 62.61 | 13.86 | 0.00 | 62.61 | 44.3 | |
| TRP | 41 | 0.00 | 0.00 | 0.00 | 0.00 | 0.0 | i |
| GLU | 42 | 19.49 | 11.64 | 0.00 | 19.49 | 13.8 | i |
| SER | 43 | 11.51 | 6.66 | 5.59 | 5.92 | 7.7 | i |
| ASN | 44 | 136.96 | 45.44 | 38.58 | 98.38 | 86.1 | o |
| GLY | 45 | 68.61 | 43.68 | 68.61 | 0.00 | 78.7 | o |
| GLN | 46 | 125.81 | 51.62 | 0.79 | 125.03 | 87.0 | o |
| PRO | 47 | 82.82 | 69.87 | 12.95 | 69.87 | 66.4 | o |
| GLU | 48 | 23.57 | 4.09 | 9.96 | 13.60 | 9.6 | i |
| ASN | 49 | 154.67 | 40.12 | 37.73 | 116.94 | 100.0 | o |
| ASN | 50 | 69.84 | 23.21 | 3.31 | 66.53 | 58.2 | o |
| TYR | 51 | 63.47 | 27.21 | 16.57 | 46.90 | 24.3 | |
| LYS | 52 | 157.61 | 112.86 | 5.40 | 152.22 | 92.5 | o |
| THR | 53 | 48.95 | 25.39 | 23.68 | 25.27 | 23.8 | |
| THR | 54 | 63.07 | 61.04 | 3.84 | 59.23 | 55.8 | o |
| PRO | 55 | 109.22 | 109.22 | 7.42 | 101.79 | 96.8 | o |
| PRO | 56 | 46.68 | 30.88 | 15.80 | 30.88 | 29.4 | |
| VAL | 57 | 76.40 | 76.40 | 4.59 | 71.81 | 58.7 | o |
| LEU | 58 | 114.86 | 96.63 | 18.23 | 96.63 | 66.1 | o |
| ASP | 59 | 48.98 | 5.83 | 9.34 | 39.64 | 35.1 | |
| SER | 60 | 123.45 | 65.29 | 42.65 | 80.79 | 100.0 | o |
| ASP | 61 | 87.54 | 53.72 | 30.83 | 56.71 | 50.2 | o |
| GLY | 62 | 38.91 | 31.91 | 38.91 | 0.00 | 44.6 | |
| SER | 63 | 1.29 | 0.00 | 0.00 | 1.29 | 1.7 | i |
| PHE | 64 | 36.04 | 36.04 | 0.00 | 36.04 | 20.0 | |
| PHE | 65 | 52.00 | 52.00 | 0.00 | 52.00 | 28.9 | |
| LEU | 66 | 1.67 | 1.67 | 0.00 | 1.67 | 1.1 | i |
| TYR | 67 | 89.25 | 55.41 | 0.35 | 88.91 | 46.0 | |
| SER | 68 | 0.00 | 0.00 | 0.00 | 0.00 | 0.0 | i |
| LYS | 69 | 95.84 | 74.80 | 0.58 | 95.26 | 57.9 | o |
| LEU | 70 | 0.03 | 0.03 | 0.00 | 0.03 | 0.0 | i |
| THR | 71 | 25.63 | 14.52 | 3.11 | 22.52 | 21.2 | |
| VAL | 72 | 3.92 | 3.92 | 3.88 | 0.04 | 0.0 | i |
| ASP | 73 | 68.40 | 12.10 | 0.79 | 67.61 | 59.8 | o |
| LYS | 74 | 60.53 | 31.65 | 0.27 | 60.26 | 36.6 | |
| SER | 75 | 51.92 | 36.67 | 1.04 | 50.87 | 65.7 | o |
| ARG | 76 | 78.71 | 47.13 | 0.19 | 78.52 | 40.2 | |
| TRP | 77 | 5.79 | 3.39 | 0.00 | 5.79 | 2.6 | i |
| GLN | 78 | 100.97 | 30.38 | 15.96 | 85.01 | 59.2 | o |
| GLN | 79 | 132.46 | 44.70 | 34.32 | 98.14 | 68.3 | o |
| GLY | 80 | 29.46 | 15.57 | 29.46 | 0.00 | 33.8 | |
| ASN | 81 | 54.55 | 11.80 | 9.35 | 45.20 | 39.5 | |
| VAL | 82 | 82.61 | 76.15 | 6.46 | 76.15 | 62.3 | o |
| PHE | 83 | 0.00 | 0.00 | 0.00 | 0.00 | 0.0 | i |
| SER | 84 | 15.68 | 2.85 | 0.00 | 15.68 | 20.3 | |
| CYS | 85 | 0.00 | 0.00 | 0.00 | 0.00 | 0.0 | i |
| SER | 86 | 4.52 | 3.63 | 0.89 | 3.63 | 4.7 | i |
| VAL | 87 | 0.65 | 0.65 | 0.65 | 0.00 | 0.0 | i |
| MET | 88 | 111.58 | 110.80 | 0.79 | 110.80 | 70.0 | o |
| HIS | 89 | 8.00 | 8.00 | 8.00 | 0.00 | 0.0 | i |
| GLU | 90 | 88.28 | 32.85 | 17.49 | 70.79 | 50.1 | o |
| ALA | 91 | 30.88 | 17.12 | 24.75 | 6.13 | 9.4 | i |
| LEU | 92 | 13.46 | 7.12 | 6.44 | 7.02 | 4.8 | i |
| HIS | 93 | 154.34 | 126.25 | 15.36 | 138.97 | 89.9 | o |
| ASN | 94 | 117.74 | 34.72 | 27.06 | 90.68 | 79.3 | o |
| HIS | 95 | 107.94 | 87.80 | 0.02 | 107.92 | 69.8 | o |
| TYR | 96 | 96.25 | 70.36 | 8.92 | 87.33 | 45.2 | |
| THR | 97 | 32.61 | 17.68 | 7.29 | 25.32 | 23.8 | |
| GLN | 98 | 83.62 | 20.27 | 18.63 | 64.99 | 45.2 | |
| LYS | 99 | 88.44 | 45.20 | 4.77 | 83.67 | 50.9 | o |
| SER | 100 | 66.87 | 30.31 | 16.66 | 50.22 | 64.9 | o |
| LEU | 101 | 17.62 | 17.62 | 5.67 | 11.95 | 8.2 | i |
| SER | 102 | 33.50 | 1.16 | 15.46 | 18.04 | 23.3 | |
| LEU | 103 | 77.59 | 71.45 | 7.26 | 70.33 | 48.1 | |
| SER | 104 | 89.76 | 44.75 | 29.35 | 60.41 | 78.0 | o |
| PRO | 105 | 190.98 | 114.92 | 49.18 | 141.81 | 100.0 | o |

CH3 sequence and modeling information. Positions are indicated with an arbitrary number. Residue GLY with number 1 corresponds to EU-number 341, residue GLN with number 2 corresponds to EU-number 342 etc until residue LEU with number 103 which has position number 443 according to EU-numbering (see IMGT table depicted in FIG. 17).

The used sequence of the CH3 region comprises a DE heterodimerization variation at positions 351 and 368 (an L371D and an L368E variation resp position 11 and 28 in the above numbering). Column In/Out indicates whether the amino acid is considered to be buried (i) or surface-exposed (o). An open space indicates a value for an amino acid that is not surface-exposed but also not buried.

TABLE 22
CH3 muts model KK variant
Probe radius: 1.400

| Residue | | Total | Apolar | Backbone | Sidechain | Ratio (%) | In/Out |
|---|---|---|---|---|---|---|---|
| GLY | 1 | 93.09 | 41.02 | 93.09 | 0.00 | 100.0 | o |
| GLN | 2 | 155.60 | 61.06 | 7.43 | 148.16 | 100.0 | o |
| PRO | 3 | 73.53 | 55.44 | 18.09 | 55.44 | 52.7 | o |
| ARG | 4 | 98.41 | 60.63 | 6.39 | 92.02 | 47.1 | |
| GLU | 5 | 85.96 | 23.95 | 5.17 | 80.79 | 57.2 | o |
| PRO | 6 | 4.18 | 0.00 | 4.18 | 0.00 | 0.0 | i |
| GLN | 7 | 73.97 | 26.02 | 5.89 | 68.08 | 47.4 | |
| VAL | 8 | 5.78 | 0.46 | 5.32 | 0.46 | 0.4 | i |
| TYR | 9 | 115.12 | 90.27 | 4.56 | 110.56 | 57.3 | o |
| THR | 10 | 31.62 | 0.00 | 20.23 | 11.39 | 10.7 | i |
| LYS | 11 | 97.36 | 87.29 | 0.02 | 97.34 | 59.2 | o |
| PRO | 12 | 54.87 | 51.32 | 3.55 | 51.32 | 48.8 | |
| PRO | 13 | 12.12 | 3.82 | 11.88 | 0.24 | 0.2 | i |
| SER | 14 | 62.86 | 50.86 | 13.37 | 49.49 | 63.9 | o |
| ARG | 15 | 202.59 | 95.58 | 10.04 | 192.55 | 98.5 | o |
| GLU | 16 | 155.43 | 59.84 | 25.05 | 130.38 | 92.3 | o |
| GLU | 17 | 45.66 | 17.81 | 3.71 | 41.96 | 29.7 | |
| MET | 18 | 42.26 | 39.82 | 2.44 | 39.82 | 25.2 | |
| THR | 19 | 130.36 | 93.70 | 34.75 | 95.61 | 90.0 | o |
| LYS | 20 | 128.39 | 82.23 | 6.69 | 121.71 | 74.0 | o |
| ASN | 21 | 132.12 | 39.02 | 14.71 | 117.41 | 100.0 | o |
| GLN | 22 | 71.88 | 17.54 | 5.70 | 66.18 | 46.1 | |
| VAL | 23 | 0.55 | 0.55 | 0.55 | 0.00 | 0.0 | i |
| SER | 24 | 27.04 | 14.16 | 3.04 | 24.01 | 31.0 | |
| LEU | 25 | 0.00 | 0.00 | 0.00 | 0.00 | 0.0 | i |
| LYS | 26 | 59.33 | 56.45 | 0.00 | 59.33 | 36.1 | |
| CYS | 27 | 0.00 | 0.00 | 0.00 | 0.00 | 0.0 | i |
| LEU | 28 | 33.08 | 33.08 | 0.00 | 33.08 | 22.6 | |
| VAL | 29 | 0.00 | 0.00 | 0.00 | 0.00 | 0.0 | i |
| LYS | 30 | 84.57 | 40.83 | 0.00 | 84.57 | 51.4 | o |
| GLY | 31 | 9.17 | 7.06 | 9.17 | 0.00 | 10.5 | i |
| PHE | 32 | 0.00 | 0.00 | 0.00 | 0.00 | 0.0 | i |
| TYR | 33 | 60.68 | 25.90 | 0.00 | 60.68 | 31.4 | |
| PRO | 34 | 41.14 | 41.14 | 0.00 | 41.14 | 39.1 | |
| SER | 35 | 35.36 | 7.78 | 1.45 | 33.92 | 43.8 | |
| ASP | 36 | 117.07 | 38.94 | 10.61 | 106.45 | 94.2 | o |
| ILE | 37 | 37.58 | 12.18 | 31.59 | 5.99 | 4.1 | i |
| ALA | 38 | 57.23 | 56.66 | 8.29 | 48.94 | 75.4 | o |
| VAL | 39 | 17.62 | 2.52 | 16.23 | 1.38 | 1.1 | i |
| GLU | 40 | 62.29 | 13.95 | 0.00 | 62.29 | 44.1 | |
| TRP | 41 | 0.00 | 0.00 | 0.00 | 0.00 | 0.0 | i |
| GLU | 42 | 19.50 | 11.41 | 0.00 | 19.50 | 13.8 | i |
| SER | 43 | 11.50 | 6.60 | 5.67 | 5.83 | 7.5 | i |
| ASN | 44 | 136.92 | 45.31 | 38.50 | 98.42 | 86.1 | o |
| GLY | 45 | 68.62 | 43.83 | 68.62 | 0.00 | 78.7 | o |
| GLN | 46 | 125.98 | 51.63 | 0.80 | 125.18 | 87.1 | o |
| PRO | 47 | 82.84 | 69.88 | 12.97 | 69.88 | 66.4 | o |
| GLU | 48 | 23.62 | 4.07 | 10.01 | 13.60 | 9.6 | i |
| ASN | 49 | 154.66 | 39.97 | 37.74 | 116.92 | 100.0 | o |
| ASN | 50 | 69.94 | 23.32 | 3.33 | 66.61 | 58.3 | o |
| TYR | 51 | 63.35 | 27.06 | 16.51 | 46.84 | 24.3 | |
| LYS | 52 | 157.10 | 112.33 | 5.40 | 151.70 | 92.2 | o |
| THR | 53 | 48.93 | 25.37 | 23.71 | 25.22 | 23.7 | |
| THR | 54 | 61.62 | 59.83 | 3.87 | 57.75 | 54.4 | o |
| PRO | 55 | 109.26 | 109.26 | 7.47 | 101.79 | 96.8 | o |
| PRO | 56 | 46.61 | 30.82 | 15.79 | 30.82 | 29.3 | |
| VAL | 57 | 76.20 | 76.20 | 4.63 | 71.57 | 58.5 | o |
| LEU | 58 | 114.88 | 96.67 | 18.21 | 96.67 | 66.1 | o |
| ASP | 59 | 48.22 | 5.78 | 9.18 | 39.04 | 34.6 | |
| SER | 60 | 123.72 | 65.51 | 43.16 | 80.56 | 100.0 | o |
| ASP | 61 | 87.28 | 54.01 | 30.73 | 56.55 | 50.0 | o |
| GLY | 62 | 39.07 | 32.04 | 39.07 | 0.00 | 44.8 | |
| SER | 63 | 1.25 | 0.00 | 0.00 | 1.25 | 1.6 | i |
| PHE | 64 | 36.09 | 36.09 | 0.00 | 36.09 | 20.0 | |
| PHE | 65 | 49.15 | 49.15 | 0.00 | 49.15 | 27.3 | |
| LEU | 66 | 1.65 | 1.65 | 0.00 | 1.65 | 1.1 | i |
| TYR | 67 | 75.47 | 46.74 | 0.29 | 75.18 | 38.9 | |
| SER | 68 | 0.00 | 0.00 | 0.00 | 0.00 | 0.0 | i |
| LYS | 69 | 95.90 | 74.90 | 0.52 | 95.38 | 58.0 | o |
| LEU | 70 | 0.03 | 0.03 | 0.00 | 0.03 | 0.0 | i |
| THR | 71 | 25.40 | 14.67 | 3.09 | 22.31 | 21.0 | |
| VAL | 72 | 4.01 | 4.01 | 3.97 | 0.04 | 0.0 | i |
| ASP | 73 | 68.35 | 12.16 | 0.76 | 67.59 | 59.8 | o |
| LYS | 74 | 60.62 | 31.68 | 0.27 | 60.35 | 36.7 | |
| SER | 75 | 52.04 | 36.69 | 1.05 | 50.99 | 65.9 | o |
| ARG | 76 | 78.94 | 47.28 | 0.18 | 78.77 | 40.3 | |
| TRP | 77 | 5.62 | 3.18 | 0.00 | 5.62 | 2.5 | i |
| GLN | 78 | 100.88 | 30.49 | 15.98 | 84.90 | 59.1 | o |
| GLN | 79 | 132.35 | 44.53 | 34.29 | 98.06 | 68.2 | o |
| GLY | 80 | 29.49 | 15.52 | 29.49 | 0.00 | 33.8 | |
| ASN | 81 | 54.67 | 11.85 | 9.34 | 45.33 | 39.7 | |
| VAL | 82 | 82.47 | 76.04 | 6.43 | 76.04 | 62.2 | o |
| PHE | 83 | 0.00 | 0.00 | 0.00 | 0.00 | 0.0 | i |
| SER | 84 | 15.48 | 2.85 | 0.00 | 15.48 | 20.0 | i |
| CYS | 85 | 0.00 | 0.00 | 0.00 | 0.00 | 0.0 | i |
| SER | 86 | 4.43 | 3.50 | 0.94 | 3.50 | 4.5 | i |
| VAL | 87 | 0.65 | 0.65 | 0.65 | 0.00 | 0.0 | i |
| MET | 88 | 111.53 | 110.73 | 0.80 | 110.73 | 69.9 | o |
| HIS | 89 | 7.99 | 7.99 | 7.99 | 0.00 | 0.0 | i |
| GLU | 90 | 88.29 | 32.90 | 17.51 | 70.78 | 50.1 | o |
| ALA | 91 | 30.79 | 17.10 | 24.66 | 6.13 | 9.4 | i |
| LEU | 92 | 13.43 | 7.16 | 6.37 | 7.06 | 4.8 | i |
| HIS | 93 | 154.33 | 126.31 | 15.44 | 138.89 | 89.8 | o |
| ASN | 94 | 117.65 | 34.62 | 26.99 | 90.66 | 79.3 | o |
| HIS | 95 | 107.74 | 87.65 | 0.01 | 107.72 | 69.7 | o |
| TYR | 96 | 95.92 | 70.08 | 8.95 | 86.97 | 45.0 | |
| THR | 97 | 32.60 | 17.86 | 7.28 | 25.32 | 23.8 | |
| GLN | 98 | 83.59 | 20.55 | 18.53 | 65.05 | 45.3 | |
| LYS | 99 | 88.47 | 45.23 | 4.85 | 83.62 | 50.8 | o |
| SER | 100 | 66.59 | 30.70 | 16.42 | 50.17 | 64.8 | o |
| LEU | 101 | 17.64 | 17.64 | 5.75 | 11.90 | 8.1 | i |
| SER | 102 | 33.57 | 1.17 | 15.51 | 18.06 | 23.3 | |
| LEU | 103 | 77.51 | 71.44 | 7.19 | 70.33 | 48.1 | |
| SER | 104 | 89.68 | 44.61 | 29.48 | 60.20 | 77.8 | o |
| PRO | 105 | 190.73 | 114.96 | 49.16 | 141.57 | 100.0 | o |

CH3 sequence and modeling information. Positions are indicated with an arbitrary number. Residue GLY with number 1 corresponds to EU-number 341, residue GLN with number 2 corresponds to EU-number 342 etc, until residue LEU with number 103 which has position number 443 according to EU-numbering (see IMGT table depicted in FIG. 17).

The used sequence of the CH3 region comprises an KK heterodimerization variation at positions 351 and 366 (an L351K and an T366K variation, resp position 11 and 26 in the above numbering). Column In/Out indicates whether the amino acid is considered to be buried (i) or surface-exposed (o). An open space indicates a value for an amino acid that is not surface-exposed.

### Example 7b: Construct Design

Non-surface and buried positions in CH2 and CH3 are varied to change the charge of multimerizing proteins incorporating these immunoglobulin regions. In total 9 exemplary variant CH2 and CH3 regions are produced and incorporated into mono, and multispecific antibodies for comparison against mono, and multispecific antibodies with wild type C2 and CH3 regions. Constructs to express heavy chain molecules comprising these separation CH2/CH3 regions are prepared similar to the methods detailed in example 1.

The amino acid variations of the tested variants are depicted in table 23.

TABLE 23
Amino acid variations (EU-numbering) in CH2 and CH3
domains of human IgG1

| | | | Charge |
|---|---|---|---|
| | | | difference |
| | | | as compared |
| | | | to a wild |
| | | | type CH |
| Domain | Residue | Variation | region |
| CH2 | V303 | V303E | −1 |
| | | V303K | +1 |
| CH3 | K370 | K370S | −1 |
| | | K370T | −1 |
| | E382 | E382Q | +1 |
| | | E382T | +1 |
| | E388 | E388L | +1 |
| | | E388M | +1 |
| | | E388T | +1 |

The variants all comprise the Fe-silent variation in the CH2-region as indicated in table 20. The variants further contain a CH3 heterodimerization domain as depicted in table 21 for the DE variant and table 22 for the KK variant. Variants that provided a negative charge increase were integrated into a DE CH3 backbone. For those that provided a positive charge increase, they were integrated into the KK CH3 backbone.

The respective heavy chains were produced with a heavy chain variable region that together with the common light chain of FIG. 13A form a variable domain that binds tetanus toxoid (TT) or that binds an extra-cellular part of c-MET. The TT variable domain has a heavy chain variable region with an amino acid sequence of MF1516. The c-MET variable domain has a heavy chain variable region with an amino acid sequence of MF3462. The amino acid sequence of the VH MF1516 and MF3462 is indicated above. Production of heavy chains with compatible heterodimerisation regions allows the preferential formation of bispecific antibodies. The heavy chain with VH MF1516 contains the DE variant CH3 domain while the heavy chain with VH3462 has the KK variant. CH3 domain.

The identity of the final constructs was confirmed by sequencing. For the production of bispecific antibodies one heavy chain contains the variable region of MF1516, a wtCH1 region and hinge region, an Fe-silent CH2 region and a DE CH3 region. The other heavy chain contains the variable region of MF3462, a wtCH1 region and hinge region, an Fe-silent CH2 region and a KK CH3 region. As mentioned above, the variants indicated in table 23 that provide a negative charge increase were integrated into the heavy chain with the DE CH3 region. Variants that provide a positive charge increase were integrated into the heavy chain with the KK CH3 region. When herein below reference is made to WT a bispecific antibody is referred to that has the above heavy and light chains but not one of the variants described in table 23.

Bispecific antibodies were produced by combining the two heavy chains. The antibodies were expressed and purified using methods that are essentially described in example 1c.

Briefly: constructs that express two heavy chains and a common light chain with the sequence of FIG. 13A were introduced into Hek293 cells. Six-day after transfection the medium of the cells was harvested. The antibody was subsequently purified from this medium with a method as described in example 1. Antibodies produced in this way are listed in FIG. 19.

Monospecific bivalent antibodies with the variants of table 23 where produced using heavy chains with CH3 domains that did not contain compatible heterodimerization CH3 domains. Heavy chains have either a VH with the amino acid sequence of MF1516 or MF3462, a wtCH1 region and hinge region, an Fc-silent CH2 region and a W1 CH3 region. The amino acid variations indicated in table 23 where introduced into either the Fe-silent CH2 region or the WT CH3 region. Variants indicated in table 23 that provide a negative charge increase were integrated into the heavy chain with the MF1516 VH region. Variants that provide a positive charge increase were integrated into the heavy chain with the MF3462 region. When herein below reference is made to WT an antibody is referred to that has the above heavy and light chains but not one of the variants described in table 23. Briefly: constructs that express the indicated heavy chain and a common light chain with the sequence of FIG. 13A were introduced into Hek293 cells. Six-day after transfection the medium of the cells was harvested. The antibody was subsequently purified from this medium with a method as described in example 1. Antibodies produced in this way are listed in FIG. 19.

### ELISA

ELISA plates were coated with c-MET, Tetanus Toxoid or Thyroglobulin for evaluating the binding the various antibodies. (c-MET (R&D systems cat #358-MT/CF) at 2.5 μg/ml. Tetanus Toxoid (Statens institute cat #T162-2 at 2 μg/ml and Thyroglobulin (Sigma Aldrich cat #T1126-500MG) at 10 μg/ml). Antibodies were incubated at 10, 1, 0.1, 0.01 μg/mL. Antibodies that bind were detected with 1:2000 diluted HRP-conjugated Protein L-based secondary antibody which binds the kappa light chain (Pierce, cat. no. 32420).

The ELISA results are summarized in FIG. 18.

Antibody PG1337 is a monospecific bivalent TT IgG1 antibody. Antibody PG1025 is a monospecific bivalent Thyroglobulin IgG1 antibody. Antibody PG2994 is a monospecific bivalent eMET IgG1 antibody. It is concluded that all bispecific antibodies bind c-Met and Tetanus Toxoid in a dose dependent manner. The bispecific antibodies do not bind to a negative control antigen (thyroglobulin). The binding does not appear to differ between antibodies that have a WT CH2/CH3 region or a variant thereof.

### EXAMPLE 8: CIEX PROFILES OF THE RESPECTIVE BISPECIFIC ANTIBODIES

CIEX experiments were performed as described in example 2. The results of the respective antibodies are depicted in FIGS. 20 and 21 and summarized in table 24.

TABLE 24
CIEX retention times of bispecific and monospecific antibodies with CH2 and CH3 separation domains. Retention
time (RT) of the respective (half)bodies are indicated as RT DEDE for the DEDE heterodimer RT PB for the bispecific
antibody, RT KK for the KK halfbody and RT KKKK for the KK heterodimer. The relative differences (ΔRT) with
respect to the DEDE and KK molecules is shown in the last two columns. All the tested variants affect the RT
on the CIEX. The direction of the shift was as expected. The best shift was observed for the V303L variant.

| | | | | | RT | RT | RT | RT | ΔRT | ΔRT |
|---|---|---|---|---|---|---|---|---|---|---|
| DE | KK | PG/PB/Po# | 1st MG'S | 2nd MG's | DEDE | PB | KK | KKKK | (PB − DEDE | (PB − KK) |
| WT | WT | PB27538p43 | MG1516C3209 | MG3462C3215 | 12.9 | 14.3 | 18.0 | 19.5 | 1.4 | 3.7 |
| WT | WT | PB27538p44 | MG1516C3209 | MG3462C3215 | 13.0 | 14.3 | 18.0 | 19.5 | 1.3 | 3.7 |
| K370S | WT | PB27538p45 | MG1516C3210 | MG3462C3215 | 12.5 | 14.1 | 17.9 | 19.5 | 1.6 | 3.9 |
| K370T | WT | PB27538p46 | MG1516C3211 | MG3462C3215 | 12.5 | 14.1 | 17.9 | 19.5 | 1.6 | 3.9 |
| V303E | WT | PB27538p47 | MG1516C3212 | MG3462C3215 | 12.3 | 13.9 | 17.9 | 19.4 | 1.6 | 4.0 |
| WT | V303K | PB27538p48 | MG1516C3209 | MG3462C3216 | 13.0 | 15.0 | 19.2 | 20.7 | 2.0 | 4.2 |
| WT | E382Q | PB27538p49 | MG1516C3209 | MG3462C3217 | 13.0 | 14.7 | 18.8 | 20.4 | 1.7 | 4.1 |
| WT | E382T | PB27538p50 | MG1516C3209 | MG3462C3218 | 13.0 | 14.7 | 18.8 | 20.4 | 1.7 | 4.1 |
| WT | E388L | PB27538p51 | MG1516C3209 | MG3462C3219 | 13.0 | 14.7 | 19.0 | 20.6 | 1.7 | 4.3 |
| WT | E388T | PB27538p52 | MG1516C3209 | MG3462C3221 | 12.9 | 14.8 | | | 1.9 | |
| WT | | PG1515p27 | MG1516C3209 | | 12.9 | | | | | |
| K370S | | PG1515p28 | MG1516C3209 | | 12.4 | | | | | |
| K370T | | PG1515p29 | MG1516C3211 | | 11.7 | | | | | |
| V303E | | PG1515p30 | MG1516C3212 | | 12.3 | | | | | |
| | WT | PG3462p29 | MG3462CS215 | | | | 17.7 | 19.3 | | |
| | V303K | PG3462p30 | MG3462CS216 | | | | 19.0 | 20.5 | | |
| | E382Q | PG3462p31 | MG3462CS217 | | | | 18.6 | 20.2 | | |
| | E382T | PG3462p32 | MG3462CS218 | | | | 18.7 | 20.2 | | |
| | E388L | PG3462p33 | MG3462CS219 | | | | 18.8 | 20.3 | | |
| | E388T | PG3462p34 | MG3462CS221 | | | | | | | |

### Example 9: Melting Temperatures of the Respective CH2 CH3 Separation Domain Containing Antibodies

Thermal stability was determined by UNCLE as explained in example 6.

TABLE 25
Uncle stability

| | | | | | | | Tagg |
|---|---|---|---|---|---|---|---|
| | | | | Tm1 | Tm2 | Tm3 | 266 |
| DE | KK | Well | Sample | (° C.) − half IgG | (° C.) − Fc part | (° C.) − Fab | (° C.) |
| WT | WT | A1 | 0.21 mg/ml PB27538p33 | ((52.7)) | 68.2 | 79.5 | 79.1 |
| WT | WT | B1 | 0.22 mg/ml PB27538p34 | | 67.9 | 79.0 | 78.6 |
| K370S | WT | C1 | 0.21 mg/ml PB27538p35 | | 68.0 | 79.0 | 78.7 |
| K370T | WT | D1 | 0.19 mg/ml PB27538p36 | | 68.5 | 78.6 | 78.6 |
| V303E | WT | E1 | 0.23 mg/ml PB27538p37 | | 66.6 | 78.2 | 77.9 |
| WT | V303K | F1 | 0.21 mg/ml PB27538p38 | | 65.5 | 78.0 | 77.6 |
| WT | E382Q | G1 | 0.2 mg/ml PB27538p39 | | 66.6 | 78.5 | 77.9 |
| WT | E382T | H1 | 0.2 mg/ml PB27538p40 | | 66.5 | 78.0 | 78.7 |
| WT | E388L | I1 | 0.19 mg/ml PB27538p41 | | 66.1 | 78.5 | 78.1 |
| WT | E388T | J1 | 0.18 mg/ml PB27538p42 | | 66.1 | 78.1 | 78.2 |
| WT | | M1 | 0.23 mg/ml PG1516p22 | 55.2 | | 78.5 | 77.7 |
| K370S | | N1 | 0.23 mg/ml PG1516p23 | 56.0 | | 78.7 | 77.9 |
| K370T | | A2 | 0.21 mg/ml PG1516p24 | 56.0 | | 79.5 | 78.8 |
| V303E | | B2 | 0.23 mg/ml PG1516p25 | 55.7 | | 79.5 | 78.6 |
| | WT | C2 | 0.17 mg/ml PG3462p22 | 54.6 | | ? | 81.0 |
| | V303K | D2 | 0.15 mg/ml PG3462p23 | 53.3 | | | 80.2 |
| | E382Q | E2 | 0.15 mg/ml PG3462p24 | 54.4 | | | 80.7 |
| | E382T | F2 | 0.18 mg/ml PG3462p25 | 55.0 | | 81.5 | 80.7 |
| | E388L | G2 | 0.14 mg/ml PG3462p26 | 54.0 | | | 80.7 |
| | E388T | N2 | 0.06 mg/ml PG3462p27 | ? | | | 83.7? |

Most bispecific antibodies with a separation variant exhibit only a modest reduction of the melting temperature (about 2-3° C.).

### TM1: Half IgG

An early TM found in half IgG and one PB which possibly is due to the half IgG in that PB preparation. TM1 is similar for all half IgG (lower for KK compared to DE half transfections; lowest for V303K)

### TM2: Melting of Fc

PBs with a variation on KK side have a reduced TM2 (by 2-3 degree)

Variation of V303 (on both, DE and KK side) causes s reduction of TM2 in the PBs

### TM3: Melting of Fab

Around ˜78-79 degree detected in all PBs as expected from the WT IgG1 controls (see FIGS. 20 and 21). This indicates that the stability of the PBs is not severely affected by the variations in the Fe

### TAGG: Same in all PBs, Higher in KK Half IgG

E388T half IgG has a high TAGG.

### Summary

TABLE 26

| | | | | | | | | Thermal stability | |
|---|---|---|---|---|---|---|---|---|---|
| | | CIEX | | | | | | | Tagg |
| PB | | RT | RT | RT | RT | ΔRT | ΔRT | Tm | 266 |
| DE | KK | DEDE | PB | KK | KKKK | (PB − DEDE) | (PB − KK) | (° C.) − Fc | (° C.) |
| WT | WT | 12.9 | 14.3 | 18.0 | 19.5 | 1.4 | 3.7 | 68.2 | 79.1 |
| WT | WT | 13.0 | 14.3 | 18.0 | 19.5 | 1.3 | 3.7 | 67.9 | 78.6 |
| K370S | WT | 12.5 | 14.1 | 17.9 | 19.5 | 1.6 | 3.9 | 68.0 | 78.7 |
| K370T | WT | 12.5 | 14.1 | 17.9 | 19.5 | 1.6 | 3.9 | 68.5 | 78.6 |
| V303E | WT | 12.3 | 13.9 | 17.9 | 19.4 | 1.6 | 4.0 | 66.6 | 77.9 |
| WT | V303K | 13.0 | 15.0 | 19.2 | 20.7 | 2.0 | 4.2 | 65.5 | 77.6 |
| WT | E382Q | 13.0 | 14.7 | 18.8 | 20.4 | 1.7 | 4.1 | 66.6 | 77.9 |
| WT | E382T | 13.0 | 14.7 | 18.8 | 20.4 | 1.7 | 4.1 | 66.5 | 78.7 |
| WT | E388L | 13.0 | 14.7 | 19.0 | 20.6 | 1.7 | 4.3 | 66.1 | 78.1 |
| WT | E388T | 12.9 | 14.8 | | | 1.9 | | 66.1 | 78.2 |

The CH2 and CH3 variants tested all favourably affect the separation of the bispecific antibody from the DEDE and KK molecules. Some variants have a higher effect. Thermal stability is only modestly affected by the separation variants. The percentage halfbody in these relatively crude preparations is also relatively constant over the respective separation variants and similar to W with the exception of E388T which has effectively 0% halfbody.

The invention provides the following aspects as part of the invention.

### ASPECTS

Aspect 1. An immunoglobulin CH1 region comprising a variation of an amino acid that is non-surface exposed in an immunoglobulin, wherein the variation is selected from
   a neutral amino acid to a negatively charged amino acid;
   a positively charged amino acid to a neutral amino acid;
   a positively charged amino acid to a negatively charged amino acid;
   a neutral amino acid to a positively charged amino acid;
   a negatively charged amino acid to a neutral amino acid; and
   a negatively charged amino acid to a positively charged amino acid.
Aspect 2. The immunoglobulin region of aspect 1, comprising two or more variations of amino acid that are non-surface exposed in an immunoglobulin.
Aspect 3. The immunoglobulin region of aspect 1 or aspect 2, which is a human immunoglobulin region.
Aspect 4. The immunoglobulin region of any one of aspects 1-3, wherein the amino acid(s) that are non-surface exposed are buried.
Aspect 5. The immunoglobulin region of any one of aspects 1-4, which is an IgG region, preferably an IgG1 region.
Aspect 6. An immunoglobulin CH1 region comprising a variation of an amino acid selected from T120, K147, D148, Y149, V154, N159, A172, Q175, S190, N201 and K213 (EU-numbering).
Aspect 7. The immunoglobulin CH1 region of aspect 6, comprising a variation of an amino acid selected from D148, Y149, V154, N159, A172, S190, and N201.
Aspect 8. The immunoglobulin CH1 region of aspect 6 or aspect 7, comprising a variation of an amino acid selected from N159 and/or N201.
Aspect 9. An antibody comprising a CH1 region of any one of aspects 1-8.
Aspect 10. The antibody of aspect, 9, comprising two or more CH1 regions of any one of aspects 1-8.
Aspect 11. The antibody of aspect 9 or aspect 10, which comprises different heavy chains.
Aspect 12. The antibody of aspect 11, which is a multispecific antibody.
Aspect 13. The multispecific antibody of aspect 11 or 12, wherein the heavy chains comprise compatible heterodimerization regions.
Aspect 14. The multispecific antibody of aspect 13, comprising compatible heterodimerization CH3 regions.
Aspect 15. The multispecific antibody of aspect 12-14 wherein one of the heavy chains comprises the CH3 variations L351D and L368E, and another of said heavy chains comprises the CH3 variations T366K and L351K.
Aspect 16. The antibody of any one of aspects 9-15, which is an IgG1 antibody.
Aspect 17. The antibody of any one of aspects 9-16, comprising one or more antibody light chains.
Aspect 18. The antibody of any one of aspects 9-17, comprising a common antibody light chain.
Aspect 19. A composition comprising the immunoglobulin region of any one of aspects 1-8 or antibody of any one of aspects 9-18.
Aspect 20. A pharmaceutical composition comprising the immunoglobulin region of any one of aspects 1-8 or antibody of any one of aspects 9-18.
Aspect 21. A nucleic acid that encodes the CH1 region of any of aspects 1-8 or antibody of any one of aspects 9-18.
Aspect 22. A nucleic acid which encodes the antibody of any one of aspects 9-18.
Aspect 23. A recombinant host cell comprising the nucleic acid of aspect 21 or aspect 22.
Aspect 24. A method of producing an antibody of any one of aspects 9-18, wherein the method comprises the steps of
   providing a nucleic acid encoding a first heavy chain with a CH1 region of any one of aspects 1-8;
   providing a nucleic acid encoding a second heavy chain, wherein said first and second heavy chain may be the same or different;
   providing a nucleic acid encoding a light chain;
   introducing said nucleic acid into host cells and culturing said host cells to express the nucleic acid(s); and producing the antibody by performing at least one of the following steps:
   collecting the antibody from the host cell culture,
   performing harvest clarification,
   performing protein capture,
   performing anion exchange chromatography, and
   performing cation exchange chromatography to separate the antibody from another antibody or an antibody fragment.
Aspect 25. A method of producing an antibody of any one of aspects 9-18, wherein the method comprises the steps of
   providing a nucleic acid encoding a first heavy chain with a CH1 region of any one of aspects 1-8;
   providing a nucleic acid encoding a second heavy chain, wherein said first and second heavy chain may be the same or different;
   providing a nucleic acid encoding a light chain;
   introducing said nucleic acid into host cells and culturing said host cells to express the nucleic acid(s); and
   collecting the antibody from the host cell culture, and
   separating the antibody from other antibodies or antibody fragments in a separation step by isoelectric focusing on a gel.
Aspect 26. The method of aspect 24 or 25, wherein said first and second heavy chains comprise compatible heterodimerization regions, preferably a compatible CH3 heterodimerization regions.
Aspect 27. A method for producing a multispecific antibody comprising a first heavy chain and a second heavy chain whose isoelectric points are different, wherein the method comprises the steps of:
   providing a nucleic acid encoding a CH1 region of the first heavy chain and a nucleic acid encoding a CH1 region of the second heavy chain, such that the isoelectric point of the first encoded heavy chain and that of the second encoded heavy chain differ, wherein at least one of said CH1 regions comprises an amino acid variation at a position selected from T120, K147, D148, Y149, V154, N159, A172, Q175, S190, N201 and K213 (EU-numbering) and
   culturing host cells to express the nucleic acid; and
   collecting the multispecific antibody from the host cell culture, using the difference in isoelectric point further comprising the steps of
   collecting the antibody from the host cell culture,
   performing harvest clarification,
   performing protein capture,
   performing anion exchange chromatography, and
   performing cation exchange chromatography to separate the antibody from another antibody or an antibody fragment.
Aspect 28. A method for purifying a multispecific antibody comprising a first heavy chain and a second heavy chain whose isoelectric points are different, wherein the method comprises the steps of:
   providing both or either one of a nucleic acid encoding a CH1 region of the first heavy chain and a nucleic acid encoding a CH1 region of the second heavy chain, such that the first encoded heavy chain and the second encoded heavy chain differ in isoelectric point, wherein at least one of said CH1 regions comprises an amino acid variation at a position selected from T120, K147, D148, Y149, V14, N159, A172, Q175, S190, N201 and K213 (EU-numbering) and
   culturing host cells to express the nucleic acid; and
   purifying the multispecific antibody from the host cell culture by isoelectric focusing and separating the multispecific antibody from another antibodies or an antibody fragment.
Aspect 29. The method of aspect 27 or aspect 28, wherein the nucleic acid encoding a homomultimer of the first heavy chain, a homomultimer of the second heavy chain, and a heteromultimer of the first and second heavy chain are expressed as proteins having different isoelectric points and produce different retention times in ion exchange chromatography.
Aspect 30. The method of any one of aspects 27-29, wherein the position(s) of said one or more amino acid variations are selected from
   a neutral amino acid to a negatively charged amino acid;
   a positively charged amino acid to a neutral amino acid;
   a positively charged amino acid to a negatively charged amino acid;
   a neutral amino acid to a positively charged amino acid;
   a negatively charged amino acid to a neutral amino acid; and
   a negatively charged amino acid to a positively charged amino acid.
Aspect 31. The method of any one of aspects 27-30, wherein the first heavy chain and the second heavy chain comprise compatible CH3 heterodimerization regions.
Aspect 32. A method of aspect 31, wherein one of said compatible CH3 heterodimerization regions comprises an L351D and L368E variation and the other comprises a T366K and 1351K variation.
Aspect 33. A CH1-containing immunoglobulin polypeptide comprising a first charged amino acid residue at position 120, position 147, position 148, position 149, position 154, position 159, position 172, position 175, position 190, position 201, or position 213.
Aspect 34. The CH1-containing immunoglobulin polypeptide of aspect 33, comprising in addition to the charged residue of aspect 33, a second charged amino acid residue at a different position selected from position 120, position 147, position 148, position 149, position 154, position 159, position 172, position 175, position 190, position 201, or position 213.
Aspect 35. A CH1-containing immunoglobulin polypeptide comprising a neutral or a negatively charged amino acid residue at position 197 and/or position 213.
Aspect 36 A CH1-containing immunoglobulin polypeptide comprising a neutral or a positively charged amino acid residue at position 159 and positively charged amino acid residue at a hinge position 216.
Aspect 37. An immunoglobulin protein comprising a first CH1-containing immunoglobulin polypeptide and a second CH1-containing immunoglobulin polypeptide, wherein the first and/or second CH1-containing immunoglobulin polypeptides comprise one or more variations of one or more amino acids selected from amino acids within the CH1 region that are non-surface exposed, such that the isoelectric point of the immunoglobulin protein comprising the first CH1-containing immunoglobulin polypeptide and the second CH1-containing immunoglobulin polypeptide is different from the isoelectric points of immunoglobulin proteins containing only the first CH1-immunoglobulin polypeptide or proteins containing only the second CH1-immunoglobulin polypeptide.
Aspect 38. An immunoglobulin protein of aspect 37, wherein said one or more variations of one or more amino acids selected from amino acids within the CH1 region are buried.
Aspect 39. A composition comprising the immunoglobulin region or antibody of any one of aspects 1-18, further comprising a variation at an amino acid selected from T197 and at a hinge position E216.
Aspect 40. An immunoglobulin protein comprising a first CH1 region containing immunoglobulin polypeptide and a second CI 1 region containing immunoglobulin polypeptide, wherein one CH1 region comprises one or more variations of an amino acid that are non-surface exposed, wherein the one or more variations of an amino acid from:
   a neutral amino acid to a negatively charged amino acid;
   a positively charged amino acid to a neutral amino acid; and
   a positively charged amino acid to a negatively charged amino acid; or:
   a neutral amino acid to a positively charged amino acid;
   a negatively charged amino acid to a neutral amino acid; and
   a negatively charged amino acid to a positively charged amino acid.
Aspect 41. An immunoglobulin protein comprising a first CH1 region containing immunoglobulin polypeptide and a second CH1 region containing immunoglobulin polypeptide, wherein one CH1 region comprises one or more variations of an amino acid that are non-surface exposed, wherein the one or more variations of an amino acid are from:
   a neutral amino acid to a negatively charged amino acid;
   a positively charged amino acid to a neutral amino acid; and
   a positively charged amino acid to a negatively charged amino acid; and the other CH1 region comprises one or more variations of an amino acid that are non-surface exposed, wherein the one or more variations of an amino acid are from:
   a neutral amino acid to a positively charged amino acid;
   a negatively charged amino acid to a neutral amino acid; and
   a negatively charged amino acid to a positively charged amino acid.
Aspect 42. An immunoglobulin protein comprising a first CH1 region containing immunoglobulin polypeptide and a second CH1 region containing immunoglobulin polypeptide, wherein the first and/or second CH1 region containing immunoglobulin polypeptides comprise one or more variations of one or more amino acids selected from amino acids within the CH1 region that are non-surface exposed, such that the iso-electric point of the immunoglobulin protein comprising the first CH region containing immunoglobulin polypeptide and the second CH1 region containing immunoglobulin polypeptide is different from the iso-electric points of immunoglobulin proteins containing only the first CH1 region immunoglobulin polypeptide and different from immunoglobulin proteins containing only the second CH1 region immunoglobulin polypeptide.
Aspect 43. The immunoglobulin protein in accordance with any one of aspects 40-42, comprising a human CH1 region.
Aspect 44. The immunoglobulin protein in accordance with any one of aspects 40-43, which is an IgG.
Aspect 45. The immunoglobulin protein in accordance with any one of aspects 40-44, wherein the amino acid(s) that are non-surface exposed are buried.
Aspect 46. The immunoglobulin protein in accordance with any one of aspects 40-44, comprising a variation of an amino acid in a CH1 region of an amino acid selected from T120, K147, D148, Y149, V154, N159, A172, Q175, S190, N201 and K213.
Aspect 47. The immunoglobulin protein in accordance with aspect 46, comprising a variation of an amino acid selected from D148, Y149, V154, N159, A172, S190, and N201.
Aspect 48. The immunoglobulin protein of aspect 47, comprising a variation of an amino acid of N159 and/or N201.
Aspect 49. The immunoglobulin protein in accordance with any one of aspects 40-48, wherein the first CH1 region containing immunoglobulin polypeptide and the second CH1 region containing immunoglobulin polypeptide are heavy chains.
Aspect 50. The immunoglobulin protein in accordance with any one of aspects 40-49, which is an antibody.
Aspect 51. The antibody of aspect 50, which is a bispecific antibody.
Aspect 52. The antibody of aspect 50, which is a multispecific antibody.
Aspect 53. An immunoglobulin protein in accordance with any one of aspects 40-52, further comprising a variation at an amino acid selected from T197 and at a hinge position E216.
Aspect 54. A composition comprising the immunoglobulin region of any one of aspects 1-8 or antibody of any one of aspects 9-18, which further comprises one or more of the following variations G122P, 1199V, N203I, S207T, and V211I.

1. An immunoglobulin CH1, CH2, CH3 region comprising a variation of an amino acid that is non-surface exposed in an immunoglobulin or a combination of said regions, wherein the variation is selected from
neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid.

2. (canceled)

3. The immunoglobulin region of claim 1, wherein said variation of an amino acid is not in the CH1/CL, CH2/CH2 domain or CH3/CH3 domain interface.

4. The immunoglobulin region of claim 1, which is an IgG region.

5. (canceled)

6. An immunoglobulin CH1 region comprising a variation of an amino acid selected from N159, N201, T120, K147, D148, Y149, V154, A172, Q175, S190, and K213 (EU-numbering);
an immunoglobulin CH2 region comprising a variation of amino acid V303; or
an immunoglobulin CH3 region comprising a variation of an amino acid selected from K370, E382 and E388.

7-9. (canceled)

10. The immunoglobulin CH1 region of claim 1, comprising two or more variations selected from:
a neutral amino acid to a negatively charged amino acid;
a positively charged amino acid to a neutral amino acid;
a positively charged amino acid to a negatively charged amino acid;
or two or more variations selected from:
a neutral amino acid to a positively charged amino acid;
a negatively charged amino acid to a neutral amino acid; and
a negatively charged amino acid to a positively charged amino acid.

11-13. (canceled)

14. The immunoglobulin region of claim 6, wherein the variation is selected from
a neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid.

15. (canceled)

16. An immunoglobulin CH1/CL domain, a CH2 domain or CH3 domain comprising an immunoglobulin region of claim 1.

17-19. (canceled)

20. An antibody comprising an immunoglobulin domain of claim 16.

21. The antibody of claim 20, comprising different heavy chains.

22. The antibody of claim 21, comprising a first heavy chain that comprises one or more variations selected from
a neutral amino acid to a negatively charged amino acid;
a positively charged amino acid to a neutral amino acid;
a positively charged amino acid to a negatively charged amino acid; and
a second heavy chain different from said first heavy chain that comprises one or more variations selected from
a neutral amino acid to a positively charged amino acid;
a negatively charged amino acid to a neutral amino acid; and
a negatively charged amino acid to a positively charged amino acid.

23. The antibody of claim 20, wherein the heavy chains comprise compatible heterodimerization regions.

24. The antibody of claim 23 comprising compatible CH3 heterodimerization regions.

25. (canceled)

26. The antibody of claim 20, comprising a first and a different second heavy chain and wherein said first heavy chain comprises one or more variations selected from
a neutral amino acid to a negatively charged amino acid;
a positively charged amino acid to a neutral amino acid; and
a positively charged amino acid to a negatively charged amino acid; of
one or more variations selected from
a neutral amino acid to a positively charged amino acid;
a negatively charged amino acid to a neutral amino acid; and
a negatively charged amino acid to a positively charged amino acid;
wherein said first heavy chain further comprises the CH3 variations L351D and L368E; and said second heavy chain comprises the CH3 variations T366K and L351K.

27. (canceled)

28. The immunoglobulin region of claim 1, comprising a CH1, a CH2 and/or a CH3 region sequence of table 14 part B.

29-32. (canceled)

33. A pharmaceutical composition comprising the immunoglobulin region, of claim 1, and a pharmaceutically acceptable excipient.

34. A nucleic acid that encodes the immunoglobulin region of claim 1.

35. A nucleic acid that encodes the antibody of claim 20.

36. A recombinant host cell comprising the nucleic acid of claim 34.

37. A method of producing an antibody, wherein the method comprises the steps of
providing nucleic acid encoding a first heavy chain with a CH1, CH2, CH3 region or combination thereof of claim 1;
providing a nucleic acid encoding a second heavy chain, wherein said first and second heavy chain may be the same or different;
providing a nucleic acid encoding a light chain;
introducing said nucleic acid into host cells and culturing said host cells to express the nucleic acid(s); and producing the antibody by performing at least one of the following steps:
collecting the antibody from the host cell culture,
performing harvest clarification,
performing protein capture,
performing anion exchange chromatography, and
performing cation exchange chromatography or isoelectric focusing to separate the antibody from another antibody or an antibody fragment.

38. (canceled)

39. The method of claim 37 wherein said first and second heavy chains comprise compatible heterodimerization regions.

40. A method for producing the antibody of claim 20, wherein the antibody is a multispecific antibody comprising a first heavy chain and a second heavy chain whose isoelectric points are different, wherein the method comprises the steps of:
providing a nucleic acid encoding a CH1, CH2, CH3 region or combination thereof of the first heavy chain and a nucleic acid encoding a CH1, CH2, CH3 region or combination thereof of the second heavy chain, such that the isoelectric point of the first encoded heavy chain and that of the second encoded heavy chain differ, wherein at least one of said CH1 regions comprises an amino acid variation at a position selected from N159, N201, T120, K147, D148, Y149, V154, A172, Q175, S190 and K213 (EU-numbering) or a CH2 region amino acid variation at position V303 (EU-numbering), or a CH3 region amino acid variation at a position selected from K370, E382 and E388 (EU-numbering) or a combination of said CH region amino acid variations, and
culturing host cells to express the nucleic acid; and
collecting the multispecific antibody from the host cell culture, using the difference in isoelectric point further comprising the steps of
collecting the antibody from the host cell culture,
performing harvest clarification,
performing protein capture,
performing anion exchange chromatography, and
performing cation exchange chromatography to separate the antibody from another antibody or an antibody fragment; or wherein the method comprises the steps of:
providing both or either one of a nucleic acid encoding a CH1, CH2, CH3 region or a combination thereof of the first heavy chain and a nucleic acid encoding a CH1, CH2, CH3 region or a combination thereof region of the second heavy chain, such that the first encoded heavy chain and the second encoded heavy chain differ in isoelectric point, wherein at least one of said regions comprises an CH1 region amino acid variation at a position selected from N159, N201, T120, K147, D148, Y149, V154, A172, 0175, S190 and K213 (EU-numbering), or a CH2 region amino acid variation at position V303 (EU-numbering), or a CH3 region amino acid variation at a position selected from K370, E382 and E388 (EU-numbering) or a combination of said CH region amino acid variations and
culturing host cells to express the nucleic acid; and
purifying the multispecific antibody from the host cell culture by isoelectric focusing and separating the multispecific antibody from another antibodies or an antibody fragment.

41-42. (canceled)

43. The method of claim 40, wherein the one or more amino acid variations are selected from
a neutral amino acid to a negatively charged amino acid;
a positively charged amino acid to a neutral amino acid;
a positively charged amino acid to a negatively charged amino acid;
a neutral amino acid to a positively charged amino acid;
a negatively charged amino acid to a neutral amino acid; and
a negatively charged amino acid to a positively charged amino acid.

44. The method of claim 40, wherein the first heavy chain and the second heavy chain comprise compatible CH3 heterodimerization regions.

45. The method of claim 43, wherein said first heavy chain comprises one or more variations selected from
a neutral amino acid to a negatively charged amino acid;
a positively charged amino acid to a neutral amino acid;
a positively charged amino acid to a negatively charged amino acid; and
said second heavy chain different from said first heavy chain that comprises one or more variations selected from
a neutral amino acid to a positively charged amino acid;
a negatively charged amino acid to a neutral amino acid; and
a negatively charged amino acid to a positively charged amino acid

46-47. (canceled)

48. A CH1-containing immunoglobulin polypeptide comprising:
a first charged amino acid residue at position 159, position 201, position 120, position 147, position 148, position 149, position 154, position 172, position 175, position 190, or position 213; or
a neutral or a negatively charged amino acid residue at position 197 and/or position 213; and/or
a neutral or a positively charged amino acid residue at position 159 and/or positively charged amino acid residue at a hinge position 216.

49. The CH1-containing immunoglobulin polypeptide of claim 48, comprising in addition to the first charged residue, a second charged amino acid residue at a different position selected from position 159, position 201, position 120, position 147, position 148, position 149, position 154, position 172, position 175, position 190, or position 213.

50-51. (canceled)

52. An immunoglobulin protein comprising a first CH-containing immunoglobulin polypeptide and a second CH1-containing immunoglobulin polypeptide, wherein the first and/or second CH1-containing immunoglobulin polypeptides comprise one or more variations of one or more amino acids selected from amino acids within the CH1 region that are non-surface exposed, such that the isoelectric point of the immunoglobulin protein comprising the first CH1-containing immunoglobulin polypeptide and the second CH1-containing immunoglobulin polypeptide is different from the isoelectric points of immunoglobulin proteins containing only the first CH1-immunoglobulin polypeptide or proteins containing only the second CH1-immunoglobulin polypeptide.

53. (canceled)

54. A composition comprising the immunoglobulin region of claim 1, further comprising a variation at an amino acid selected from T197 and at a hinge position E216.

55. The immunoglobulin protein of claim 52, wherein one CH1 region comprises one or more variations of an amino acid that are non-surface exposed, wherein the one or more variations of an amino acid from:
a neutral amino acid to a negatively charged amino acid;
a positively charged amino acid to a neutral amino acid; and
a positively charged amino acid to a negatively charged amino acid; or:
a neutral amino acid to a positively charged amino acid;
a negatively charged amino acid to a neutral amino acid; and
a negatively charged amino acid to a positively charged amino acid; or wherein the one or more variations of an amino acid are from:
a neutral amino acid to a negatively charged amino acid;
a positively charged amino acid to a neutral amino acid; and
a positively charged amino acid to a negatively charged amino acid;
and the other CH1 region comprises one or more variations of an amino acid that are non-surface exposed, wherein the one or more variations of an amino acid are from:
a neutral amino acid to a positively charged amino acid;
a negatively charged amino acid to a neutral amino acid; and
a negatively charged amino acid to a positively charged amino acid.

56-64. (canceled)

65. The immunoglobulin protein in accordance with claim 52, which is an antibody.

66-69. (canceled)

70. A CH2-containing immunoglobulin polypeptide comprising a charged amino acid residue at position 303 or a non-charged amino acid residue at a position selected from position 370, position 382, or position 388.

71-75. (canceled)

76. A composition comprising the immunoglobulin region of claim 1, which comprises one or more of the following variations: G122P, I199V, N203L, S207T, and V211I.


SEQUENCE LISTING (72 sequences)

SEQ ID NO: 1 - protein, 107 aa, Artificial Sequence
  Synthetic Peptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 2 - protein, 107 aa, Artificial Sequence [sequence omitted]

SEQ ID NO: 3 - DNA, 321 bp, Artificial Sequence
  DNA sequence of IgV 1-39*01/IGJ 1*01
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 4 - DNA, 321 bp, Artificial Sequence
  DNA sequence light chain constant region (CL)
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 5 - protein, 30 aa, Artificial Sequence
  Synthetic Peptide
EVQLVESGGGVVQPGRSLRLSCAASGFTFS

SEQ ID NO: 6 - protein, 5 aa, Artificial Sequence
  CDR1 heavy Chain variable region
SYGMH

SEQ ID NO: 7 - protein, 14 aa, Artificial Sequence
  FW2 heavy Chain variable region
WVRQAPGKGLEWVA

SEQ ID NO: 8 - protein, 17 aa, Artificial Sequence
  CRD2 heavy Chain variable region
VISYDGSNKYYADSVKG

SEQ ID NO: 9 - protein, 32 aa, Artificial Sequence
  CDR2 heavy Chain variable region
RFTISRDNSKNTLYLQMNSLRAEDTAVYYCAR

SEQ ID NO: 10 - protein, 10 aa, Artificial Sequence
  Synthetic Peptide
ALFTTIAMDY

SEQ ID NO: 11 - protein, 8 aa, Artificial Sequence
  Synthetic Peptide
WGQGTLVT

SEQ ID NO: 12 - protein, 119 aa, Artificial Sequence
  Synthetic Peptide
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVTVSS

SEQ ID NO: 13 - protein, 30 aa, Artificial Sequence
  Synthetic Peptide
EVQLVETGGGVVQPGRSLRLSCAASGFTFS

SEQ ID NO: 14 - protein, 5 aa, Artificial Sequence
  Synthetic Peptide
QYAMH

SEQ ID NO: 15 - protein, 14 aa, Artificial Sequence
  Synthetic Peptide
WVRQAPGKGLEWVA

SEQ ID NO: 16 - protein, 17 aa, Artificial Sequence
  Synthetic Peptide
IISHDERNKYYVDSGMG

SEQ ID NO: 17 - protein, 32 aa, Artificial Sequence
  Synthetic Peptide
RFTISRDNSKNTLFLQMNSLRSEDTAVYYCAR

SEQ ID NO: 18 - protein, 13 aa, Artificial Sequence
  Synthetic Peptide
DMRKGGYYYGFDV

SEQ ID NO: 19 - protein, 8 aa, Artificial Sequence
  Synthetic Peptide
WGQGTTVT

SEQ ID NO: 20 - protein, 122 aa, Artificial Sequence
  Synthetic Peptide
EVQLVETGGGVVQPGRSLRLSCAASGFTFSQYAMHWVRQAPGKGLEWVAIISHDERNKYYVDSGMGRFTISRDNSKNTLFLQMNSLRSEDTAVYYCARDMRKGGYYYGFDVWGQGTTVTVSS

SEQ ID NO: 21 - protein, 30 aa, Artificial Sequence
  Synthetic Peptide
EVQLLESGGGLVQPGGSLRLSCAASGFTFS

SEQ ID NO: 22 - protein, 5 aa, Artificial Sequence
  Synthetic Peptide
SYAMS

SEQ ID NO: 23 - protein, 14 aa, Artificial Sequence
  Synthetic Peptide
WVRQAPGKGLEWVS

SEQ ID NO: 24 - protein, 17 aa, Artificial Sequence
  Synthetic Peptide
AISGSGGSTYYADSVKG

SEQ ID NO: 25 - protein, 32 aa, Artificial Sequence; identical to SEQ ID NO: 9
  Synthetic Peptide

SEQ ID NO: 26 - protein, 12 aa, Artificial Sequence
  Synthetic Peptide
GKSHYSWDAFDY

SEQ ID NO: 27 - protein, 11 aa, Artificial Sequence
  Synthetic Peptide
WGQGTLVTVSS

SEQ ID NO: 28 - protein, 121 aa, Artificial Sequence
  Synthetic Peptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGKSHYSWDAFDYWGQGTLVTVSS

SEQ ID NO: 29 - protein, 127 aa, Artificial Sequence
  Synthetic Peptide
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSS

SEQ ID NO: 30 - protein, 96 aa, Artificial Sequence
  Synthetic Peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDK

SEQ ID NO: 31 - protein, 110 aa, Artificial Sequence
  Synthetic Peptide
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 32 - protein, 110 aa, Artificial Sequence
  Synthetic Peptide
APELGRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 33 - protein, 106 aa, Artificial Sequence
  Synthetic Peptide
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 98 aa, Artificial Sequence
  Synthetic Peptide
ASTKGPSVFPLAPSSKSTSGGTAALGCLVKDYFPEPVTVSWNSGALTSGVHTFPAVLQSSGLYSLSSVVTVPSSSLGTQTYICKVNHKPSNTKVDKRV

SEQ ID NO: 35 - protein, 98 aa, Artificial Sequence
  Synthetic Peptide
ASTKGPSVFPLAPSSKSTSGGTAALGCLVKDYFPEPVTVSWNSGALTSGVHTFPAVLQSSGLYSLSSVVTVPSSSLGTQTYICDVNHKPSNTKVDKRV

SEQ ID NO: 36 - protein, 98 aa, Artificial Sequence
  Synthetic Peptide
ASTKGPSVFPLAPSSKSTSGGTAALGCLVKDYFPEPVTVSWNSGALTSGVHTFPPVLQSSGLYSLSSVVTVPASSLGTQTYICKVNHKPSNTKVDKRV

SEQ ID NO: 37 - protein, 98 aa, Artificial Sequence
  Synthetic Peptide
ASTKGPSVFPLAPSSKSTSGGTAALGCLVKDYFPEPVTVSWNSGALTSGVHTFPPVLQSSGLYSLSSVVTVPASSLGTQTYICDVNHKPSNTKVDKRV

SEQ ID NO: 38 - protein, 98 aa, Artificial Sequence
  Synthetic Peptide
ASKKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 39 - protein, 98 aa, Artificial Sequence
  Synthetic Peptide
ASDKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 40 - protein, 98 aa, Artificial Sequence
  Synthetic Peptide
ASTKGPSVFPLAPSSKSTSGGTAALGCLVKDYFPEPVTVSWNSGALTSGVHTFPAVLQSSGLYSLSSVVTVPSSSLGTQDYICNVNHKPSNTKVDQRV

SEQ ID NO: 41 - protein, 98 aa, Artificial Sequence
  Synthetic Peptide
ASTKGPSVFPLAPSSKSTSGGTAALGCLVKKYFPEPVTVSWNSGALTSGVHTFPAVLKSSGLYSLSSVVTVPSSSLGTQTYICNVNHKPSNTKVDKRV

SEQ ID NO: 42 - protein, 98 aa, Artificial Sequence
  Synthetic Peptide
ASTKGPSVFPLAPSSKSTSGGTAALGCLVKDYFPEPVTVSWKSGALTSGVHTFPAVLQSSGLYSLSSVVTVPSSSLGTQTYICNVNHKPSNTKVDKRV

SEQ ID NO: 43 - protein, 98 aa, Artificial Sequence
  Synthetic Peptide
ASTKGPSVFPLAPSSKSTSGGTAALGCLVKDYFPEPVTVSWDSGALTSGVHTFPAVLQSSGLYSLSSVVTVPSSSLGTQTYICNVNHKPSNTKVDKRV

SEQ ID NO: 44 - protein, 98 aa, Artificial Sequence
  Synthetic Peptide
ASTKGPSVFPLAPSSKSTSGGTAALGCLVKDYFPEPVTVSWDSGALTSGVHTFPAVLQSSGLYSLSSVVTVPSSSLGTQTYICNVNHKPSNTKVDQRV

SEQ ID NO: 45 - protein, 98 aa, Artificial Sequence
  Synthetic Peptide
ASTKGPSVFPLAPSSKSTSGGTAALGCLVEDYFPEPVTVSWNSGALTSGVHTFPAVLESSGLYSLSSVVTVPSSSLGTQTYICNVNHKPSNTKVDKRV

SEQ ID NO: 46 - protein, 98 aa, Artificial Sequence
  Synthetic Peptide
ASTKGPSVFPLAPSSKSTSGGTAALGCLVKDAFPEPITVSWNSGALTSGVHTFPPVLQSSGLYSLSSVVTVPASSLGTQTYICNVNHKPSNTKVDKRV

SEQ ID NO: 47 - protein, 98 aa, Artificial Sequence
  Synthetic Peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDQRV

SEQ ID NO: 48 - protein, 98 aa, Artificial Sequence
  Synthetic Peptide
ASTKGPSVFPLAPSSKSTSGGTAALGCLVKDYFPEPVTVSWNSGALTSGVHTFPAVLQSSGLYSLSSVVTVPSSSLGTQTYICDVNHKPSNTKVDQRV

SEQ ID NO: 49 - protein, 98 aa, Artificial Sequence
  Synthetic Peptide
ASKKGPSVFPLAPSSKSTSGGTAALGCLVKDYFPEPVTVSWNSGALTSGVHTFPAVLQSSGLYSLSSVVTVPSSSLGTQTYICKVNHKPSNTKVDKRV

SEQ ID NO: 50 - protein, 98 aa, Artificial Sequence
  Synthetic Peptide
ASTKGPSVFPLAPSSKSTSGGTAALGCLVKDYFPEPVTVSWKSGALTSGVHTFPAVLQSSGLYSLSSVVTVPSSSLGTQTYICKVNHKPSNTKVDKRV

SEQ ID NO: 51 - protein, 98 aa, Artificial Sequence
  Synthetic Peptide
ASKKGPSVFPLAPSSKSTSGGTAALGCLVKDYFPEPVTVSWKSGALTSGVHTFPAVLQSSGLYSLSSVVTVPSSSLGTQTYICNVNHKPSNTKVDKRV

SEQ ID NO: 52 - protein, 98 aa, Artificial Sequence
  Synthetic Peptide
ASKKGPSVFPLAPSSKSTSGGTAALGCLVKDYFPEPVTVSWKSGALTSGVHTFPAVLQSSGLYSLSSVVTVPSSSLGTQTYICKVNHKPSNTKVDKRV

SEQ ID NO: 53 - protein, 98 aa, Artificial Sequence
  Synthetic Peptide
ASTKGPSVFPLAPSSKSTSGGTAALGCLVKDYFPEPVTVSWDSGALTSGVHTFPAVLQSSGLYSLSSVVTVPSSSLGTQTYICDVNHKPSNTKVDKRV

SEQ ID NO: 54 - protein, 98 aa, Artificial Sequence
  Synthetic Peptide
ASTKGPSVFPLAPSSKSTSGGTAALGCLVKDYFPEPVTVSWDSGALTSGVHTFPAVLQSSGLYSLSSVVTVPSSSLGTQTYICDVNHKPSNTKVDQRV

SEQ ID NO: 55 - protein, 110 aa, Artificial Sequence
  Synthetic Peptide
APELLGGPSVFLFPPKPKDTLMISRTPEVTCVVVDVSHEDPEVKFNWYVDGVEVHNAKTKPREEQYNSTYRVESVLTVLHQDWLNGKEYKCKVSNKALPAPIEKTISKAK

SEQ ID NO: 56 - protein, 110 aa, Artificial Sequence
  Synthetic Peptide
APELLGGPSVFLFPPKPKDTLMISRTPEVTCVVVDVSHEDPEVKFNWYVDGVEVHNAKTKPREEQYNSTYRVKSVLTVLHQDWLNGKEYKCKVSNKALPAPIEKTISKAK

SEQ ID NO: 57 - protein, 110 aa, Artificial Sequence
  Synthetic Peptide
APELGRGPSVFLFPPKPKDTLMISRTPEVTCVVVDVSHEDPEVKFNWYVDGVEVHNAKTKPREEQYNSTYRVESVLTVLHQDWLNGKEYKCKVSNKALPAPIEKTISKAK

SEQ ID NO: 58 - protein, 110 aa, Artificial Sequence
  Synthetic Peptide
APELGRGPSVFLFPPKPKDTLMISRTPEVTCVVVDVSHEDPEVKFNWYVDGVEVHNAKTKPREEQYNSTYRVKSVLTVLHQDWLNGKEYKCKVSNKALPAPIEKTISKAK

SEQ ID NO: 59 - protein, 106 aa, Artificial Sequence
  Synthetic Peptide
GQPREPQVYTLPPSREEMTKNQVSLTCLVSGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 106 aa, Artificial Sequence
  Synthetic Peptide
GQPREPQVYTLPPSREEMTKNQVSLTCLVTGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 106 aa, Artificial Sequence
  Synthetic Peptide
GQPREPQVYTLPPSREEMTKNQVSLTCLVKGFYPSDIAVEWQSNGQPENNYKTTPPVLDSDGSFFLYSKLTVDKSRWQQGNVFSCSVMHEALHNHYTQKSLSLSPG

SEQ ID NO: 62 - protein, 106 aa, Artificial Sequence
  Synthetic Peptide
GQPREPQVYTLPPSREEMTKNQVSLTCLVKGFYPSDIAVEWTSNGQPENNYKTTPPVLDSDGSFFLYSKLTVDKSRWQQGNVFSCSVMHEALHNHYTQKSLSLSPG

SEQ ID NO: 63 - protein, 106 aa, Artificial Sequence
  Synthetic Peptide
GQPREPQVYTLPPSREEMTKNQVSLTCLVKGFYPSDIAVEWESNGQPLNNYKTTPPVLDSDGSFFLYSKLTVDKSRWQQGNVFSCSVMHEALHNHYTQKSLSLSPG

SEQ ID NO: 64 - protein, 106 aa, Artificial Sequence
  Synthetic Peptide
GQPREPQVYTLPPSREEMTKNQVSLTCLVKGFYPSDIAVEWESNGQPMNNYKTTPPVLDSDGSFFLYSKLTVDKSRWQQGNVFSCSVMHEALHNHYTQKSLSLSPG

SEQ ID NO: 65 - protein, 106 aa, Artificial Sequence
  Synthetic Peptide
GQPREPQVYTLPPSREEMTKNQVSLTCLVKGFYPSDIAVEWESNGQPTNNYKTTPPVLDSDGSFFLYSKLTVDKSRWQQGNVFSCSVMHEALHNHYTQKSLSLSPG

SEQ ID NO: 66 - protein, 106 aa, Artificial Sequence
  Synthetic Peptide
GQPREPQVYTKPPSREEMTKNQVSLKCLVKGFYPSDIAVEWQSNGQPENNYKTTPPVLDSDGSFFLYSKLTVDKSRWQQGNVFSCSVMHEALHNHYTQKSLSLSPG

SEQ ID NO: 67 - protein, 106 aa, Artificial Sequence
  Synthetic Peptide
GQPREPQVYTKPPSREEMTKNQVSLKCLVKGFYPSDIAVEWTSNGQPENNYKTTPPVLDSDGSFFLYSKLTVDKSRWQQGNVFSCSVMHEALHNHYTQKSLSLSPG

SEQ ID NO: 68 - protein, 106 aa, Artificial Sequence
  Synthetic Peptide
GQPREPQVYTKPPSREEMTKNQVSLKCLVKGFYPSDIAVEWESNGQPLNNYKTTPPVLDSDGSFFLYSKLTVDKSRWQQGNVFSCSVMHEALHNHYTQKSLSLSPG

SEQ ID NO: 69 - protein, 106 aa, Artificial Sequence
  Synthetic Peptide
GQPREPQVYTKPPSREEMTKNQVSLKCLVKGFYPSDIAVEWESNGQPMNNYKTTPPVLDSDGSFFLYSKLTVDKSRWQQGNVFSCSVMHEALHNHYTQKSLSLSPG

SEQ ID NO: 70 - protein, 106 aa, Artificial Sequence
  Synthetic Peptide
GQPREPQVYTKPPSREEMTKNQVSLKCLVKGFYPSDIAVEWESNGQPTNNYKTTPPVLDSDGSFFLYSKLTVDKSRWQQGNVFSCSVMHEALHNHYTQKSLSLSPG

SEQ ID NO: 71 - protein, 106 aa, Artificial Sequence
  Synthetic Peptide
GQPREPQVYTDPPSREEMTKNQVSLTCEVSGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 106 aa, Artificial Sequence
  Synthetic Peptide
GQPREPQVYTDPPSREEMTKNQVSLTCEVTGFYPSDIAVEWESNGQPENNYKTTPPVLDSDGSFFLYSKLTVDKSRWQQGNVFSCSVMHEALHNHYTQKSLSLSPG